SEQUENCE LISTING (156 sequences)

SEQ ID NO: 1 - protein, 1288 aa, Severe acute respiratory syndrome coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 2 - DNA, 3864 bp, Severe acute respiratory syndrome coronavirus
  source 1..3864: mol_type = genomic DNA
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacatggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccggagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcgcctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctgggtctgcttcctctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggaccctccagaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaag

SEQ ID NO: 3-4 - skipped (2 entries)

SEQ ID NO: 5 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - DNA, 3822 bp, Severe acute respiratory syndrome coronavirus 2
  source 1..3822: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 7-10 - skipped (4 entries)

SEQ ID NO: 11 - protein, 26 aa, synthetic construct
EVQLVESGGGLVKPGGSLRLSCAASG

SEQ ID NO: 12 - protein, 9 aa, synthetic construct
FEFGSYEMN

SEQ ID NO: 13 - protein, 14 aa, synthetic construct
WVRQAPGKGLEWVS

SEQ ID NO: 14 - protein, 17 aa, synthetic construct
SISEDGYTTYYPDSLKG

SEQ ID NO: 15 - protein, 30 aa, synthetic construct
RFTISRDSAKNSLYLQMNSLRADDTAVYYC

SEQ ID NO: 16 - protein, 17 aa, synthetic construct
ARDFGGDTNWAGTGFTY

SEQ ID NO: 17 - protein, 11 aa, synthetic construct
WGQGTLVTVSS

SEQ ID NO: 18 - DNA, 372 bp, synthetic construct
  source 1..372: mol_type = other DNA
gaagtacagctggtggagtcaggaggaggtctggtcaaaccaggaggatcacttaggctgagctgcgctgcctctggctttgaatttgggtcttatgaaatgaattgggttcgacaagcacctggaaagggcctcgaatgggtttcctctatctctgaagatggatacacaacctattaccccgactcactcaaaggccgattcacaatttcccgagattccgctaaaaactcactgtatctgcaaatgaatagcctaagggccgatgacacagccgtgtactattgtgcccgcgactttggcggggacactaattgggctggaaccggctttacttactggggacagggaacactcgtgaccgtttcaagc

SEQ ID NO: 19 - protein, 124 aa, synthetic construct
EVQLVESGGGLVKPGGSLRLSCAASGFEFGSYEMNWVRQAPGKGLEWVSSISEDGYTTYYPDSLKGRFTISRDSAKNSLYLQMNSLRADDTAVYYCARDFGGDTNWAGTGFTYWGQGTLVTVSS

SEQ ID NO: 20 - skipped

SEQ ID NO: 21 - protein, 22 aa, synthetic construct
QSVLTQPPSVSGAPGQRITISC

SEQ ID NO: 22 - protein, 14 aa, synthetic construct
EGSSSNIGAGYDVH

SEQ ID NO: 23 - protein, 15 aa, synthetic construct
WYQQLPGTAPKLLIY

SEQ ID NO: 24 - protein, 7 aa, synthetic construct
GSSVRNY

SEQ ID NO: 25 - protein, 32 aa, synthetic construct
GVPDRFSGSKSGTSASLAITGLQAEDEADYYC

SEQ ID NO: 26 - protein, 12 aa, synthetic construct
QSYDSDLGILYT

SEQ ID NO: 27 - protein, 10 aa, synthetic construct
FGTGTKVTVL

SEQ ID NO: 28 - DNA, 336 bp, synthetic construct
  source 1..336: mol_type = other DNA
cagagcgttttaacccaaccgccaagcgtctctggtgcaccgggtcaaagaatcactatctcctgtgaaggatcctcttcaaatattggggctggctatgatgtgcactggtatcagcaactacctgggaccgctcctaagctgctgatttatggaagtagtgttcgcaactatggagtgccggacagattcagtggatccaaatctggaaccagcgcctccctcgctatcaccggcctgcaagccgaggatgaggccgactactactgccaaagttacgactctgacttgggcatcctgtacaccttcgggaccggcaccaaggtgactgtgttg

SEQ ID NO: 29 - protein, 112 aa, synthetic construct
QSVLTQPPSVSGAPGQRITISCEGSSSNIGAGYDVHWYQQLPGTAPKLLIYGSSVRNYGVPDRFSGSKSGTSASLAITGLQAEDEADYYCQSYDSDLGILYTFGTGTKVTVL

SEQ ID NO: 30 - skipped

SEQ ID NO: 31 - protein, 26 aa, synthetic construct
EVQLVESGGGLVKPGGSLRLSCAASG

SEQ ID NO: 32 - protein, 9 aa, synthetic construct
FTFGSYEMN

SEQ ID NO: 33 - protein, 14 aa, synthetic construct
WVRQAPGKGLEWVS

SEQ ID NO: 34 - protein, 17 aa, synthetic construct
SISEDGYSTYYPDSLKG

SEQ ID NO: 35 - protein, 30 aa, synthetic construct; identical to SEQ ID NO: 15

SEQ ID NO: 36 - protein, 17 aa, synthetic construct
ARDFGGDTAWAGTGFTY

SEQ ID NO: 37 - protein, 11 aa, synthetic construct
WGQGTLVTVSS

SEQ ID NO: 38 - DNA, 372 bp, synthetic construct
  source 1..372: mol_type = other DNA
gaggtgcagttagtcgaaagcggtggaggacttgttaaaccaggagggtcactaaggttgagttgcgcggcttcaggattcaccttcggatcatacgaaatgaactgggttcgtcaagcgccgggaaaaggcttggagtgggtcagtagtatctcagaggatggatacagcacgtattatcctgattccttgaaaggtcgttttacaataagtagggattccgctaaaaactcattgtatcttcaaatgaactccttgagggctgacgacacggctgtgtattactgcgcgagagactttggtggcgacactgcgtgggcgggaacgggttttacctactggggacagggcacccttgttacagtgtcaagt

SEQ ID NO: 39 - protein, 124 aa, synthetic construct
EVQLVESGGGLVKPGGSLRLSCAASGFTFGSYEMNWVRQAPGKGLEWVSSISEDGYSTYYPDSLKGRFTISRDSAKNSLYLQMNSLRADDTAVYYCARDFGGDTAWAGTGFTYWGQGTLVTVSS

SEQ ID NO: 40 - skipped

SEQ ID NO: 41 - protein, 22 aa, synthetic construct
QSVLTQPPSVSGAPGQRITISC

SEQ ID NO: 42 - protein, 14 aa, synthetic construct
TGSSSNIGAGYDVH

SEQ ID NO: 43 - protein, 15 aa, synthetic construct
WYQQLPGTAPKLLIY

SEQ ID NO: 44 - protein, 7 aa, synthetic construct
GSSSRNY

SEQ ID NO: 45 - protein, 32 aa, synthetic construct; identical to SEQ ID NO: 25

SEQ ID NO: 46 - protein, 12 aa, synthetic construct
QSYDSDLGVLYT

SEQ ID NO: 47 - protein, 10 aa, synthetic construct
FGTGTKVTVL

SEQ ID NO: 48 - DNA, 336 bp, synthetic construct
  source 1..336: mol_type = other DNA
caatctgtacttacacagcctcccagcgtgagcggtgctccaggacagaggatcactatttcctgtactggcagttcatccaatattggtgcaggatatgacgttcattggtaccaacaacttccgggaaccgccccaaaacttttaatctatggcagcagtagtcgtaattatggtgtgcctgataggttctccggctctaaaagcggtaccagtgcgagtttagccatcacagggctacaagccgaggatgaggccgattattattgtcaaagctacgatagcgatctaggcgttttgtacaccttcggtacaggcacgaaagtcactgttctt

SEQ ID NO: 49 - protein, 112 aa, synthetic construct
QSVLTQPPSVSGAPGQRITISCTGSSSNIGAGYDVHWYQQLPGTAPKLLIYGSSSRNYGVPDRFSGSKSGTSASLAITGLQAEDEADYYCQSYDSDLGVLYTFGTGTKVTVL

SEQ ID NO: 50 - skipped

SEQ ID NO: 51 - protein, 26 aa, synthetic construct
EVQLVESGGGLVKPGGSLRLSCAASG

SEQ ID NO: 52 - protein, 9 aa, synthetic construct
FTFSSYYMN

SEQ ID NO: 53 - protein, 14 aa, synthetic construct
WVRQAPGKGLEWVS

SEQ ID NO: 54 - protein, 17 aa, synthetic construct
SISEDGYSTYYPDSLKG

SEQ ID NO: 55 - protein, 30 aa, synthetic construct; identical to SEQ ID NO: 15

SEQ ID NO: 56 - protein, 17 aa, synthetic construct
ARDFSGHTAWAGTGFEY

SEQ ID NO: 57 - protein, 11 aa, synthetic construct
WGQGTLVTVSS

SEQ ID NO: 58 - DNA, 372 bp, synthetic construct
  source 1..372: mol_type = other DNA
gaggtgcagctggtggagtctgggggaggcctggtcaagcctggggggtccctgagactgtcctgtgcagcctctggattcaccttcagtagctattatatgaactgggtcaggcaggctccagggaaggggctggagtgggtctcatccattagtgaggacggatacagcacctactaccccgactcactgaagggcagattcaccatctccagagacagcgccaagaactcactgtatctgcaaatgaacagcctgagagccgatgacacggctgtgtattactgtgccagagatttttcaggccacacggcgtgggcaggaacgggatttgagtactggggccaaggaaccctggtcaccgtctcctca

SEQ ID NO: 59 - protein, 124 aa, synthetic construct
EVQLVESGGGLVKPGGSLRLSCAASGFTFSSYYMNWVRQAPGKGLEWVSSISEDGYSTYYPDSLKGRFTISRDSAKNSLYLQMNSLRADDTAVYYCARDFSGHTAWAGTGFEYWGQGTLVTVSS

SEQ ID NO: 60 - skipped

SEQ ID NO: 61 - protein, 22 aa, synthetic construct
QSVLTQPPSVSGAPGQRITISC

SEQ ID NO: 62 - protein, 14 aa, synthetic construct
TGSSSNIGAGYDVH

SEQ ID NO: 63 - protein, 15 aa, synthetic construct
WYQQLPGTAPKLLIY

SEQ ID NO: 64 - protein, 7 aa, synthetic construct
GSSSRNS

SEQ ID NO: 65 - protein, 32 aa, synthetic construct; identical to SEQ ID NO: 25

SEQ ID NO: 66 - protein, 12 aa, synthetic construct
QSYDSSLSVLYT

SEQ ID NO: 67 - protein, 10 aa, synthetic construct
FGTGTKVTVL

SEQ ID NO: 68 - DNA, 336 bp, synthetic construct
  source 1..336: mol_type = other DNA
cagtctgtgctgacgcagccgccctcagtgtctggggccccagggcagaggatcaccatctcctgcactgggagcagctccaacatcggggcaggttatgatgtacactggtaccagcagcttccaggaacagcccccaaacttctgatctatggttctagctccaggaactcaggggtccctgacaggttctctggctccaagtctggcacctcagcctccctggccatcactgggcttcaggctgaggatgaggctgattattactgccaaagttatgactctagtctgtccgtcttgtatacgttcggaactgggaccaaggtcaccgtcctg

SEQ ID NO: 69 - protein, 112 aa, synthetic construct
QSVLTQPPSVSGAPGQRITISCTGSSSNIGAGYDVHWYQQLPGTAPKLLIYGSSSRNSGVPDRFSGSKSGTSASLAITGLQAEDEADYYCQSYDSSLSVLYTFGTGTKVTVL

SEQ ID NO: 70 - skipped

SEQ ID NO: 71 - protein, 26 aa, synthetic construct
EVQLVESGGGLVKPGGSLRLSCAASG

SEQ ID NO: 72 - protein, 9 aa, synthetic construct
FDFGSYEMN

SEQ ID NO: 73 - protein, 14 aa, synthetic construct
WVRQAPGKGLEWVS

SEQ ID NO: 74 - protein, 17 aa, synthetic construct
SISEDGYTTYYPDSLKG

SEQ ID NO: 75 - protein, 30 aa, synthetic construct; identical to SEQ ID NO: 15

SEQ ID NO: 76 - protein, 17 aa, synthetic construct
ARDFGGDTNWAGTGFTY

SEQ ID NO: 77 - protein, 11 aa, synthetic construct
WGQGTLVTVSS

SEQ ID NO: 78 - DNA, 372 bp, synthetic construct
  source 1..372: mol_type = other DNA
gaggtgcagttagtcgaaagcggtggaggacttgttaaaccaggagggtcactaaggttgagttgcgcggcttcaggattcgatttcggatcatacgaaatgaactgggttcgtcaagcgccgggaaaaggcttggagtgggtcagtagtatctcagaggatggatacacgacgtattatcctgattccttgaaaggtcgttttacaataagtagggattccgctaaaaactcattgtatcttcaaatgaactccttgagggctgacgacacggctgtgtattactgcgcgagagactttggtggcgacactaattgggcgggaacgggttttacctactggggacagggcacccttgttacagtgtcaagt

SEQ ID NO: 79 - protein, 124 aa, synthetic construct
EVQLVESGGGLVKPGGSLRLSCAASGFDFGSYEMNWVRQAPGKGLEWVSSISEDGYTTYYPDSLKGRFTISRDSAKNSLYLQMNSLRADDTAVYYCARDFGGDTNWAGTGFTYWGQGTLVTVSS

SEQ ID NO: 80 - skipped

SEQ ID NO: 81 - protein, 22 aa, synthetic construct
QSVLTQPPSVSGAPGQRITISC

SEQ ID NO: 82 - protein, 14 aa, synthetic construct
TGTSSNIGAGYDVH

SEQ ID NO: 83 - protein, 15 aa, synthetic construct
WYQQLPGTAPKLLIY

SEQ ID NO: 84 - protein, 7 aa, synthetic construct
GSSVRNY

SEQ ID NO: 85 - protein, 32 aa, synthetic construct; identical to SEQ ID NO: 25

SEQ ID NO: 86 - protein, 12 aa, synthetic construct
QSYDSDLGVLYT

SEQ ID NO: 87 - protein, 10 aa, synthetic construct
FGTGTKVTVL

SEQ ID NO: 88 - DNA, 336 bp, synthetic construct
  source 1..336: mol_type = other DNA
caatctgtacttacacagcctcccagcgtgagcggtgctccaggacagaggatcactatttcctgtactggcacatcatccaatattggtgcaggatatgacgttcattggtaccaacaacttccgggaaccgccccaaaacttttaatctatggcagcagtgttcgtaattatggtgtgcctgataggttctccggctctaaaagcggtaccagtgcgagtttagccatcacagggctacaagccgaggatgaggccgattattattgtcaaagctacgatagcgatctaggcgttttgtacaccttcggtacaggcacgaaagtcactgttctt

SEQ ID NO: 89 - protein, 112 aa, synthetic construct
QSVLTQPPSVSGAPGQRITISCTGTSSNIGAGYDVHWYQQLPGTAPKLLIYGSSVRNYGVPDRFSGSKSGTSASLAITGLQAEDEADYYCQSYDSDLGVLYTFGTGTKVTVL

SEQ ID NO: 90 - skipped

SEQ ID NO: 91 - protein, 26 aa, synthetic construct
EVQLVESGGGLVKPGGSLRLSCAASG

SEQ ID NO: 92 - protein, 9 aa, synthetic construct
FEFGSYEMN

SEQ ID NO: 93 - protein, 14 aa, synthetic construct
WVRQAPGKGLEWVS

SEQ ID NO: 94 - protein, 17 aa, synthetic construct
SISEDGYTTYYPDSLKG

SEQ ID NO: 95 - protein, 30 aa, synthetic construct; identical to SEQ ID NO: 15

SEQ ID NO: 96 - protein, 17 aa, synthetic construct
ARDFGGDTNWAGTGFTY

SEQ ID NO: 97 - protein, 11 aa, synthetic construct
WGQGTLVTVSS

SEQ ID NO: 98 - DNA, 372 bp, synthetic construct
  source 1..372: mol_type = other DNA
gaggtgcagttagtcgaaagcggtggaggacttgttaaaccaggagggtcactaaggttgagttgcgcggcttcaggattcgagttcggatcatacgaaatgaactgggttcgtcaagcgccgggaaaaggcttggagtgggtcagtagtatctcagaggatggatacacgacgtattatcctgattccttgaaaggtcgttttacaataagtagggattccgctaaaaactcattgtatcttcaaatgaactccttgagggctgacgacacggctgtgtattactgcgcgagagactttggtggcgacactaattgggcgggaacgggttttacctactggggacagggcacccttgttacagtgtcaagt

SEQ ID NO: 99 - protein, 124 aa, synthetic construct; identical to SEQ ID NO: 19

SEQ ID NO: 100 - skipped

SEQ ID NO: 101 - protein, 22 aa, synthetic construct
QSVLTQPPSVSGAPGQRITISC

SEQ ID NO: 102 - protein, 14 aa, synthetic construct
TGSSSNIGAGYDVH

SEQ ID NO: 103 - protein, 15 aa, synthetic construct
WYQQLPGTAPKLLIY

SEQ ID NO: 104 - protein, 7 aa, synthetic construct
GSSLRNY

SEQ ID NO: 105 - protein, 32 aa, synthetic construct; identical to SEQ ID NO: 25

SEQ ID NO: 106 - protein, 12 aa, synthetic construct
QSYDSDLGVLYT

SEQ ID NO: 107 - protein, 10 aa, synthetic construct
FGTGTKVTVL

SEQ ID NO: 108 - DNA, 336 bp, synthetic construct
  source 1..336: mol_type = other DNA
caatctgtacttacacagcctcccagcgtgagcggtgctccaggacagaggatcactatttcctgtactggcagttcatccaatattggtgcaggatatgacgttcattggtaccaacaacttccgggaaccgccccaaaacttttaatctatggcagcagtctgcgtaattatggtgtgcctgataggttctccggctctaaaagcggtaccagtgcgagtttagccatcacagggctacaagccgaggatgaggccgattattattgtcaaagctacgatagcgatctaggcgttttgtacaccttcggtacaggcacgaaagtcactgttctt

SEQ ID NO: 109 - protein, 112 aa, synthetic construct
QSVLTQPPSVSGAPGQRITISCTGSSSNIGAGYDVHWYQQLPGTAPKLLIYGSSLRNYGVPDRFSGSKSGTSASLAITGLQAEDEADYYCQSYDSDLGVLYTFGTGTKVTVL

SEQ ID NO: 110 - skipped

SEQ ID NO: 111 - protein, 26 aa, synthetic construct
EVQLVESGGGLVKPGGSLRLSCAASG

SEQ ID NO: 112 - protein, 9 aa, synthetic construct
FEFGSYEMN

SEQ ID NO: 113 - protein, 14 aa, synthetic construct
WVRQAPGKGLEWVS

SEQ ID NO: 114 - protein, 17 aa, synthetic construct
SISEDGYTTYYPDSLKG

SEQ ID NO: 115 - protein, 30 aa, synthetic construct; identical to SEQ ID NO: 15

SEQ ID NO: 116 - protein, 17 aa, synthetic construct
ARDFGGDTNWAGTGFTY

SEQ ID NO: 117 - protein, 11 aa, synthetic construct
WGQGTLVTVSS

SEQ ID NO: 118 - DNA, 372 bp, synthetic construct; identical to SEQ ID NO: 98
  source 1..372: mol_type = other DNA

SEQ ID NO: 119 - protein, 124 aa, synthetic construct; identical to SEQ ID NO: 19

SEQ ID NO: 120 - skipped

SEQ ID NO: 121 - protein, 22 aa, synthetic construct
QSVLTQPPSVSGAPGQRITISC

SEQ ID NO: 122 - protein, 14 aa, synthetic construct
EGSSSNIGAGYDVH

SEQ ID NO: 123 - protein, 15 aa, synthetic construct
WYQQLPGTAPKLLIY

SEQ ID NO: 124 - protein, 7 aa, synthetic construct
GSSERNY

SEQ ID NO: 125 - protein, 32 aa, synthetic construct; identical to SEQ ID NO: 25

SEQ ID NO: 126 - protein, 12 aa, synthetic construct
QSYDSDLGVLYT

SEQ ID NO: 127 - protein, 10 aa, synthetic construct
FGTGTKVTVL

SEQ ID NO: 128 - DNA, 336 bp, synthetic construct
  source 1..336: mol_type = other DNA
caatctgtacttacacagcctcccagcgtgagcggtgctccaggacagaggatcactatttcctgtgaaggcagttcatccaatattggtgcaggatatgacgttcattggtaccaacaacttccgggaaccgccccaaaacttttaatctatggcagcagtgagcgtaattacggtgtgcctgataggttctccggctctaaaagcggtaccagtgcgagtttagccatcacagggctacaagccgaggatgaggccgattattattgtcaaagctacgatagcgatctaggcgttttgtacaccttcggtacaggcacgaaagtcactgttctt

SEQ ID NO: 129 - protein, 112 aa, synthetic construct
QSVLTQPPSVSGAPGQRITISCEGSSSNIGAGYDVHWYQQLPGTAPKLLIYGSSERNYGVPDRFSGSKSGTSASLAITGLQAEDEADYYCQSYDSDLGVLYTFGTGTKVTVL

SEQ ID NO: 130 - skipped

SEQ ID NO: 131 - protein, 26 aa, synthetic construct
EVQLVESGGGLVKPGGSLRLSCAASG

SEQ ID NO: 132 - protein, 9 aa, synthetic construct
FDFGSYEMN

SEQ ID NO: 133 - protein, 14 aa, synthetic construct
WVRQAPGKGLEWVS

SEQ ID NO: 134 - protein, 17 aa, synthetic construct
SISEDGRSTYYPDSLKG

SEQ ID NO: 135 - protein, 30 aa, synthetic construct; identical to SEQ ID NO: 15

SEQ ID NO: 136 - protein, 17 aa, synthetic construct
ARDFGGDTAWAGTGFTY

SEQ ID NO: 137 - protein, 11 aa, synthetic construct
WGQGTLVTVSS

SEQ ID NO: 138 - DNA, 372 bp, synthetic construct
  source 1..372: mol_type = other DNA
gaggtgcagttagtcgaaagcggtggaggacttgttaaaccaggagggtcactaaggttgagttgcgcggcttcaggattcgatttcggatcatacgaaatgaactgggttcgtcaagcgccgggaaaaggcttggagtgggtcagtagtatctcagaggatggacgaagcacgtattatcctgattccttgaaaggtcgttttacaataagtagggattccgctaaaaactcattgtatcttcaaatgaactccttgagggctgacgacacggctgtgtattactgcgcgagagactttggtggcgacactgcgtgggcgggaacgggttttacctactggggacagggcacccttgttacagtgtcaagt

SEQ ID NO: 139 - protein, 124 aa, synthetic construct
EVQLVESGGGLVKPGGSLRLSCAASGFDFGSYEMNWVRQAPGKGLEWVSSISEDGRSTYYPDSLKGRFTISRDSAKNSLYLQMNSLRADDTAVYYCARDFGGDTAWAGTGFTYWGQGTLVTVSS

SEQ ID NO: 140 - skipped

SEQ ID NO: 141 - protein, 22 aa, synthetic construct
QSVLTQPPSVSGAPGQRITISC

SEQ ID NO: 142 - protein, 14 aa, synthetic construct
TGSSSNIGAGYDVH

SEQ ID NO: 143 - protein, 15 aa, synthetic construct
WYQQLPGTAPKLLIY

SEQ ID NO: 144 - protein, 7 aa, synthetic construct
GSSSRNY

SEQ ID NO: 145 - protein, 32 aa, synthetic construct; identical to SEQ ID NO: 25

SEQ ID NO: 146 - protein, 12 aa, synthetic construct
QSYDSDLGVLYT

SEQ ID NO: 147 - protein, 10 aa, synthetic construct
FGTGTKVTVL

SEQ ID NO: 148 - DNA, 336 bp, synthetic construct; identical to SEQ ID NO: 48
  source 1..336: mol_type = other DNA

SEQ ID NO: 149 - protein, 112 aa, synthetic construct; identical to SEQ ID NO: 49

SEQ ID NO: 150 - skipped

SEQ ID NO: 151 - protein, 9 aa, synthetic construct
  VARIANT 2: note = X is E or D
FXFGSYEMN

SEQ ID NO: 152 - protein, 17 aa, synthetic construct
  VARIANT 7: note = X is Y or R
  VARIANT 8: note = X is T or S
SISEDGXXTYYPDSLKG

SEQ ID NO: 153 - protein, 17 aa, synthetic construct
  VARIANT 9: note = X is N or A
ARDFGGDTXWAGTGFTY

SEQ ID NO: 154 - protein, 14 aa, synthetic construct
  VARIANT 1: note = X is E or T
  VARIANT 3: note = X is S or T
XGXSSNIGAGYDVH

SEQ ID NO: 155 - protein, 7 aa, synthetic construct
  VARIANT 4: note = X is V, L, E or S
GSSXRNY

SEQ ID NO: 156 - protein, 12 aa, synthetic construct
  VARIANT 9: note = X is I or V
QSYDSDLGXLYT

(19) United States
(12) Patent Application Publication
(10) Pub. No.: US 2026/0103506 A1
(43) Pub. Date: Apr. 16, 2026

(54) SARS-COV2 ANTIBODIES AND METHODS OF USE THEREOF

(71) Applicant: Invivyd, Inc., Waltham, MA (US)

(72) Inventors: Laura Walker, Waltham, MA (US); Anna Wec, Waltham, MA (US); Robert Allen, Newton, MA (US); Brandyn West, Jamestown, NY (US); Daniel Chupp, Boston, MA (US)

(21) Appl. No.: 19/256,409

(22) Filed: Jul. 1, 2025

Related U.S. Application Data
(60) Provisional application No. 63/666,293, filed on Jul. 1, 2024.
(60) Provisional application No. 63/692,466, filed on Sep. 9, 2024.
(60) Provisional application No. 63/743,455, filed on Jan. 9, 2025.
(60) Provisional application No. 63/748,729, filed on Jan. 23, 2025.
(60) Provisional application No. 63/752,445, filed on Jan. 31, 2025.
(60) Provisional application No. 63/766,024, filed on Mar. 3, 2025.
(60) Provisional application No. 63/806,136, filed on May 15, 2025.
(60) Provisional application No. 63/830,559, filed on Jun. 26, 2025.

(51) Int. Cl. C07K 16/104; A61K 39/00; A61P 37/04

(52) U.S. Cl. CPC C07K 16/104; A61P 37/04; A61K 2039/505; A61K 2039/545; C07K 2317/33; C07K 2317/76; C07K 2317/92; C07K 2317/94

(57) ABSTRACT

The present disclosure is directed to antibodies and antigen binding fragments thereof, having improved binding specificity for coronaviruses, such as SARS-CoV-2, including neutralizing antibodies. Other embodiments contemplate using anti-CoV-S antibodies, and binding fragments thereof, for the diagnosis, assessment, and treatment of diseases and disorders associated with coronaviruses or the S protein thereof and conditions where neutralization or inhibition of coronaviruses or the S protein thereof would be therapeutically beneficial.

### RELATED APPLICATIONS

This application claims the benefit of priority to U.S. Provisional Application No. 63/666,293, filed on Jul. 1, 2024, U.S. Provisional Application No. 63/692,466, filed on Sep. 9, 2024, U.S. Provisional Application No. 63/743,455, filed on Jan. 9, 2025, U.S. Provisional Application No. 63/748,729, filed on Jan. 23, 2025, U.S. Provisional Application No. 63/752,445, filed on Jan. 31, 2025, U.S. Provisional Application No. 63/766,024, filed on Mar. 3, 2025, U.S. Provisional Application No. 63/806,136, filed on May 15, 2025, and U.S. Provisional Application No. 63/830,559, filed on Jun. 26, 2025. The entire contents of each of the foregoing applications are incorporated hereby by reference.

### SEQUENCE LISTING

This application contains a Sequence Listing which has been submitted electronically in XML format and is hereby incorporated by reference in its entirety. The XML copy, created on Jun. 30, 2025, is named 132280-01009.xml and is 143,025 bytes in size.

### BACKGROUND

Coronaviruses (“CoV”) are genetically classified into four major genera: the Alphacoronavirus genus (ACoV genus); the Betacoronavirus genus (BCoV genus); the Gammacoronavirus genus (CCoV genus); and Deltacoronavirus genus (DCoV genus), and while ACoV and BCoV primarily infect mammals CCoV and DCoV predominantly infect birds (Wu A. et al., Cell Host Microbe. 2020 Mar. 11; 27(3):325-328). Coronaviruses that infect humans were first identified in the mid-1960s, and currently, seven confirmed CoV species are known as human pathogens. Four CoV species, the HCoV-HKU1 and HCoV-OC43 from the BCoV genus and the HCoV-229E and HCoV-NL63 from the ACoV genus, are endemic species in humans and cause mild respiratory symptoms, mostly in pediatric patients (Brielle E. S., et al., BioRxiv reprint, 2020.03.10). The other three human CoV species, the SARS-CoV, the MERS-CoV, and the SARS-CoV-2 (also known as “2019-nCoV”), all of which are from the BCoV genus, have caused severe outbreaks, including the Severe Acute Respiratory Syndrome (SARS) outbreak in 2002-2003, the Middle East Respiratory Syndrome (MERS) outbreak in 2012-2013, and the current (2019-) pandemic of the coronavirus disease of 2019 (“COVID-19”).

The genome of coronaviruses, whose size ranges between approximately 26,000 and 32,000 bases, includes a variable number (from 6 to 11) of open reading frames (“ORFs”) (Wu A. et al., Cell Host Microbe. 2020; 27(3):325-328). The first ORF encodes 16 non-structural proteins (“nsps”), and the remaining ORFs encode accessory proteins and structural proteins. The four major structural proteins are the spike surface glycoprotein (“S protein” or “S” or “spike protein”), small envelope protein (“E protein” or “E”), matrix protein (“M protein” or “M”), and nucleocapsid protein (“N protein”, or “N”).

The S protein, which plays an essential role in binding to receptors on the host cell and determines host tropism (Zhu Z. et al., Infect Genet Evol. 2018; 61:183-184), forms homotrimers protruding from the viral surface (Li F. Annu Rev Virol. 2016 Sep. 29; 3(1):237-261. Epub 2016 Aug. 25). The S protein is processed into two non-covalently associated subunits, S1 and S2, and each monomer in the trimeric S assembly is a heterodimer of S1 and S2 subunits. Cryo-EM studies have revealed that the S1 subunit is comprised of four domains: an N-terminal domain (NTD), a C-terminal domain (CTD), and two subdomains (Walls A. C. et al., Nature 531, 114-117 (2016).; Tortorici M. A. and Veesler D., Adv Virus Res. 2019; 105:93-116. doi: 10.1016/bs.aivir.2019.08.002. Epub 2019 Aug. 22.; Wrapp D. et al., Science 367, 1260-1263 (2020)). The CTD functions as the receptor-binding domain (RBD) for both SARS-CoV and SARS-CoV-2 (Li F. J Virol. 2015 February; 89(4):1954-64. doi: 10.1128/JVI.02615-14. Epub 2014 Nov. 26). The S2 subunit contains the fusion peptide, heptad repeat 1 and 2, and a transmembrane domain, all of which are required to mediate fusion of the viral and host cell membranes.

SARS-CoV and SARS-CoV-2 bind to and use angiotensin-converting enzyme 2 (ACE2) of a host cell as a receptor to enter the host cells (Ge X. Y. et al., Nature. 2013 Nov. 28; 503(7477):535-8; Hoffmann M. et al., Cell. 2020 Mar. 4). The motif within the RBD that particularly binds to RCE2 is often referred to as the “ACE2-binding motif”. SARS-CoV can also use CD209L (also known as L-SIGN) as an alternative receptor (Jeffers S. A. et al., Proc Natl Acad Sci USA. 2004 Nov. 2; 101(44):15748-53. Epub 2004 Oct. 20). In contrast, MERS-CoV binds dipeptidyl peptidase 4 (“DPP4”, also known as CD26) of the host cell via a different RBD of the S protein.

Cell entry of coronaviruses often depends also on priming of the S protein by host cell proteases. Recently, SARS-CoV-2 was found to use the serine protease TMPRSS2 for S protein priming and ACE2 for entry (Wu A. et al., Cell Host Microbe. 2020; 27(3):325-328; Hoffmann M. et al., Cell. 2020 Mar. 4).

The genome of SARS-CoV-2 is about 29.8 kb nucleotides and encodes 15 non-structural proteins, four structural proteins (S, E, M, and N) and eight accessory proteins (3a, 3b, p6, 7a, 7b, 8b, 9b, and orf14) (Wu A. et al., Cell Host Microbe. 2020 Mar. 11; 27(3):325-328). While SARS-CoV-2 is genetically close to a SARS-like bat CoV and also to SARS-CoV, a number of sequence differences have been identified. When SARS-CoV-2 is compared to SARS-CoV or SARS-like bat CoV, 380 amino acid differences or substitutions were found, 27 of which are in the S protein, including 6 substitutions in the RBD at amino acid region 357-528 (but not in the receptor-binding motifs that directly interact with ACE2) and 6 substitutions in the underpinning subdomain (SD) at amino acid region 569-655.

Multiple variants of SARS-CoV-2 have developed since the onset of COVID. Among those, the Omicron variant is a highly divergent variant with a high number of mutations, including 26-32 mutations in the spike protein, some of which are associated with humoral immune escape potential and higher transmissibility. Omicron is a much more rapidly spreading variant that has grown from first report on Nov. 25, 2021 to a record global spike of cases that is already several times the level of Delta. Moreover, Omicron is causing an increase in breakthrough infections among fully vaccinated individuals. As a result, the protection provided by both vaccination and prior infection is much reduced.

Accordingly, there is an unmet need for development of new treatment, e.g., antibodies, with improved properties, for treating SAR-CoV-2, in particular, new variants of concern (VOCs) as they emerge.

### SUMMARY

The present disclosure is based on the surprising discovery of a new antibody with multiple improved properties, e.g., improved in vitro neutralization efficacy across all SARS-CoV-2 variants of concern (VOCs) described to date, higher binding affinity, and more favorable thermal and colloidal stability in vitro, as compared to other existing antibodies to date. Indeed, the new antibody is notably more advantageous, as its improved thermal and colloidal stability in vitro leads to a more stable product at higher concentrations and higher temperatures, and an improved shelf life. Moreover, this new antibody exhibited broad and improved potency against various variants of SARS-CoV-2, including Omicron sublineages such as XBB.1.5, JN.1, KP.3.1.1, XEC, and LP.8.1. The improved stability, shelf life, and higher potency allow for longer dosing intervals, lower doses, and smaller dosing volumes. These improvements to potency and half-life represent a step forward in the potential clinical utility of such an antibody via either immediate delivery of extraordinarily high antiviral titer in a treatment setting, or long-term delivery of attractive preventive titers in a desirable dose and administration form. This antibody represents a more promising candidate for therapeutic development and provides a framework for the development of vaccines that induce broadly neutralizing antibody responses.

Accordingly, in one aspect, the present disclosure provides an isolated antibody, or antigen-binding fragment thereof, that binds to the spike protein of a corona virus (CoV-S), wherein said antibody, or antigen-binding fragment thereof, comprises a heavy chain variable region (VH) and a light chain variable region (VL), wherein the VH comprises an amino acid sequence having at least 90%, 91%, 92%, 93%, 94%, 95%, 96%, 97%, 98%, or 99% sequence identity to, comprises, or consists of an amino acid sequence of SEQ ID NO: 19, and wherein the VL comprises an amino acid sequence having at least 90%, 91%, 92%, 93%, 94%, 95%, 96%, 97%, 98%, or 99% sequence identity to, comprises, or consists of an amino acid sequence of SEQ ID NO: 29.

In some embodiments, the VH comprises a VH CDR1 comprising the amino acid sequence FX₁FGSYEMN of SEQ ID NO: 151, wherein X₁ is E or D; a VH CDR2 comprising the amino acid sequence SISEDGX₁X₂TYYPDSLKG of SEQ ID NO: 152, wherein X₁ is Y or R, and X₂ is T or S; and a VH CDR3 comprising the amino acid sequence ARDFGGDTX₁WAGTGFTY of SEQ ID NO: 153, wherein X₁ is N or A; and the VL comprises a VL CDR1 comprising the amino acid sequence X₁GX₂SSNIGAGYDVH of SEQ ID NO: 154, wherein X₁ is E or T, and X₂ is S or T; a VL CDR2 comprising the amino acid sequence GSSX₁RNY of SEQ ID NO: 155, wherein X₁ is V, L, E or S; and a VL CDR3 comprising the amino acid sequence QSYDSDLGX₁LYT of SEQ ID NO: 156, wherein X₁ is I or V.

In some embodiments, the VH comprises a VH CDR1 comprising SEQ ID NO: 12, a VH CDR2 comprising SEQ ID NO: 14, and a VH CDR3 comprising SEQ ID NO: 16, and the VL comprises a VL CDR1 comprising SEQ ID NO: 22, a VL CDR2 comprising SEQ ID NO: 24, and a VL CDR3 comprising SEQ ID NO: 26.

In some embodiments, the VH comprises a VH CDR1 comprising SEQ ID NO: 72, a VH CDR2 comprising SEQ ID NO: 74, and a VH CDR3 comprising SEQ ID NO: 76, and the VL comprises a VL CDR1 comprising SEQ ID NO: 82, a VL CDR2 comprising SEQ ID NO: 84, and a VL CDR3 comprising SEQ ID NO: 86.

In some embodiments, the VH comprises a VH CDR1 comprising SEQ ID NO: 92, a VH CDR2 comprising SEQ ID NO: 94, and a VH CDR3 comprising SEQ ID NO: 96, and the VL comprises a VL CDR1 comprising SEQ ID NO: 102, a VL CDR2 comprising SEQ ID NO: 104, and a VL CDR3 comprising SEQ ID NO: 106.

In some embodiments, the VH comprises a VH CDR1 comprising SEQ ID NO: 112, a VH CDR2 comprising SEQ ID NO: 114, and a VH CDR3 comprising SEQ ID NO: 116, and the VL comprises a VL CDR1 comprising SEQ ID NO: 122, a VL CDR2 comprising SEQ ID NO: 124, and a VL CDR3 comprising SEQ ID NO: 126.

In some embodiments, the VH comprises a VH CDR1 comprising SEQ ID NO: 132, a VH CDR2 comprising SEQ ID NO: 134, and a VH CDR3 comprising SEQ ID NO: 136, and the VL comprises a VL CDR1 comprising SEQ ID NO: 142, a VL CDR2 comprising SEQ ID NO: 144, and a VL CDR3 comprising SEQ ID NO: 146.

In one aspect, the present disclosure provides an isolated antibody, or antigen-binding fragment thereof, which specifically binds to the spike protein of a coronavirus (“CoV-S”), wherein said antibody, or antigen-binding fragment thereof, comprises a heavy chain variable region (VH) comprising a VH CDR1 comprising SEQ ID NO: 12, a VH CDR2 comprising SEQ ID NO: 14, and a VH CDR3 comprising SEQ ID NO: 16, and a light chain variable region (VL) comprising a VL CDR1 comprising SEQ ID NO: 22, a VL CDR2 comprising SEQ ID NO: 24, and a VL CDR3 comprising SEQ ID NO: 26.

In some embodiments, the isolated antibody, or antigen-binding fragment thereof, comprises a VH comprising SEQ ID NO: 19 and a VL comprising SEQ ID NO: 29.

In some embodiments, the isolated antibody, or antigen-binding fragment thereof, comprises a VH consisting of SEQ ID NO: 19 and a VL consisting of SEQ ID NO: 29.

In some embodiments, the VH comprises the VH amino acid sequence of VYD2311, and the VL comprises the VL amino acid sequence of VYD2311.

In some embodiments, the antibody or antibody-binding fragment thereof comprises the VH CDR1, VH CDR2, VH CDR3, VL CDR1, VL CDR2, and VL CDR3 amino acid sequences of VYD2311.

In some embodiments, SARS-CoV-S comprises a sequence having at least 95% identity to the amino acid sequence of SEQ ID NO: 1, and wherein SARS-CoV-2-S comprises a sequence having at least 95% identity to the amino acid sequence of SEQ ID NO:5.

In some embodiments, the isolated antibody, or antigen-binding fragment thereof, cross-reacts with SARS-CoV-S and SARS-CoV-2-S.

In certain embodiments, an antibody or antigen-binding fragment thereof is capable of binding to a SARS-CoV-2 variant. In some embodiments, the SARS-CoV-2-S is a B.1.1.7 variant (alpha), a B. 1.351 variant (beta), a B.1.1.28 variant, a B. 1.429 variant, a P.1 variant, a B.1.617 variant (e.g., B.1.617.1 and B.1.617.2 (delta)), a C.37 variant, a 1.621 variant, a AY.1 variant, a 1.623 variant, a C.36 variant, a A.27 variant, a AV.1 variant, a B.1.1.482 variant, a B.1.1.523 variant, a B.1.427 variant, a AY.4 variant, a AY.11 variant, a D614G variant, or a B.1.1.529/BA.1 variant (also known as the Omicron variant) or its sub-lineages (e.g., BA1.1, BA.4/5, BA.4.6, BA.7, BQ.1, BQ.1.1, BA.2, BA.2.75, BN.1, XBB, XBB.1, XBB.1.5, BJ.1, BM.1.1.1, BA.2.3.20, BF.7, XBC, CH.1.1, XBB.1.16, XBB.1.5.10, XBB.1.5.1, XBB.2.3, FL.1.5.1, XBB.1.5.CONV0817, XBB.1.5.70, BA.2.86, HV.1, HK.3, JN.1, JN.4, JD.1.1, BA.2, JN.1.11.1, GE.1.2.1, JN.1.13.1, KQ.1, KP.1.1, KP.3, KP.2, JN.1.50, KP.3.1.1, XEC, LB.1, MV.1, LP.8.1, MC.10.2, LF.7, LP.8.1.2, FL.15.1.1, or LF.7.2.1).

In some embodiments, the antibody, or antigen-binding fragment thereof, cross-reacts with SARS-CoV-S and SARS-CoV-2-S.

In some embodiments, the antibody, or antigen-binding fragment thereof, binds to the receptor binding domain (RBD) or the N-terminal domain (NTD) of SARS-CoV-S and/or of SARS-CoV-2-S.

In some embodiments, the antibody, or antigen-binding fragment thereof, binds to the S1 subunit and/or the S2 subunit of SARS-CoV-S and/or of SARS-CoV-2-S.

In some embodiments, the antibody, or antigen-binding fragment thereof, binds to the ACE2-binding motif of SARS-CoV-S and/or of SARS-CoV-2-S.

In one embodiment, the antibody, or antigen-binding fragment thereof, competes with ACE2.

In some embodiments, the antibody, or antigen-binding fragment thereof, (a) binds to the S protein of SARS-CoV and/or of SARS-CoV-2; and (b) does not bind to any of the S proteins of HCoV-229E, HCoV-HKU1, HCoV-NL63, and HCoV-OC43.

In some embodiments, the antibody, or antigen-binding fragment thereof, (a) binds to the S protein of SARS-CoV and/or of SARS-CoV-2; and (b) binds to the S protein of at least one of HCoV-229E, HCoV-HKU1, HCoV-NL63, and HCoV-OC43.

In one embodiment, the antibody, or antigen-binding fragment thereof, binds to CoV-S with a KD value of: (i) about 100 nM or lower; (ii) about 20 nM or lower; (iii) about 1 nM or lower; (iv) about 100 pM or lower; (v) about 10 pM or lower; (vi) about 1 pM or lower; or (vii) about 0.1 pM or lower.

In some embodiments, the antibody, or antigen-binding fragment thereof, binds to the CoV-S from the XBB.1.5 variant, the BQ.1 variant, the BA.2 variant, the BA.4/5 variant, or the BA.2.86 variant of SARS-CoV-2 with a KD value of about 20 nM or lower, or about 10 nM or lower, about 5 nM or lower, about 2 nM or lower, or about 1 nM or lower.

In some embodiments, the antibody, or antigen-binding fragment thereof, binds to the CoV-S from the XBB.1.5 variant of SARS-CoV-2 with a KD value of about 1 nM or lower.

In some embodiments, the antibody, or antigen-binding fragment thereof, binds to the CoV-S from the BA.2.86 variant of SARS-CoV-2 with a KD value of about 2 nM or lower.

In some embodiments, the dissociation constant (KD) is measured using an assay selected from the group consisting of surface plasmon resonance (SPR), ELISAs, radioimmunoassays, Western blotting, and bio-layer interferometry (BLI). In one embodiment, KD is measured by SPR. In another embodiment, KD is measured by ELISA. In one embodiment, KD is measured by BLI.

In some embodiments, the antibody, or antigen-binding fragment thereof, neutralizes SARS-CoV and/or SARS-CoV-2. In some embodiments, the neutralization activity is measured using a vesicular stomatitis virus (VSV)-based pseudovirus system. In some embodiments, the neutralization activity is measured using a murine leukemia virus (MLV)-based pseudovirus system. In some embodiments, the neutralization activity is measured using a PhenoSense SARS-CoV-2 pseudovirus neutralizing assay.

In some embodiments, the antibody, or antigen-binding fragment thereof, neutralizes SARS-CoV and/or SARS-CoV-2 at: (i) an IC50 of about 100 nM or lower, of about 50 nM or lower, of about 20 nM or lower, of about 10 nM or lower, of about 5 nM or lower, of about 2 nM or lower, of about 1 nM or lower, of about 500 pM or lower, of about 200 pM or lower, of about 100 pM or lower, of about 50 pM or lower, of about 20 pM or lower, of about 10 pM or lower, of about 5 pM or lower, of about 2 pM or lower, or of about 1 pM or lower; and/or (ii) an IC50 of about 1 μg/mL or lower, of about 500 ng/mL or lower, of about 200 ng/mL or lower, of about 100 ng/mL or lower, of about 50 ng/mL or lower, of about 20 ng/mL or lower, of about 10 ng/mL or lower, of about 5 ng/mL or lower, of about 2 ng/mL or lower, or of about 1 ng/mL or lower, in vitro. In some embodiments, the neutralization activity is measured using a VSV-based pseudovirus system. In some embodiments, the neutralization activity is measured using a MLV-based pseudovirus system. In some embodiments, the neutralization activity is measured using a PhenoSense SARS-CoV-2 pseudovirus neutralizing assay.

In some embodiments, the antibody, or antigen-binding fragment thereof, neutralizes the BA.1 variant, the XBB.1.5 variant, the XBB.1.16 variant, the XBB.1.5.10 variant, the XBB.1.5.1 variant, the XBB.2.3 variant, the FL.1.5.1 variant, the XBB.1.5.CONV0817 variant, the BA.2.86 variant, the HV.1 variant, the HK.3 variant, the JN.1 variant, the JN.4 variant, the JD.1.1 variant, the BA.2 variant, the GE.1.2.1 variant, the JN.1.13.1 variant, the KQ.1 variant, the KP.1.1 variant, the KP.3 variant, the KP.2 variant, the JN.1.50 variant, the KP.3.1.1 variant, the XEC variant, the LB.1 variant, the MV.1 variant, the LP.8.1 variant, the MC.10.2. variant, the LF.8 variant, the LP.8.1.2 variant, the FL.15.1.1 variant, or the LF.7.2.1 variant of SARS-CoV-2 with an IC50 of about 2000 ng/mL or lower, of about 1500 ng/mL or lower, of about 1000 ng/mL or lower, of about 500 ng/mL or lower, of about 100 ng/mL or lower, of about 50 ng/mL or lower, of about 40 ng/mL or lower, of about 30 ng/mL or lower, of about 20 ng/mL or lower, of about 10 mg/mL or lower, of about 5 ng/mL or lower, of about 2 ng/mL or lower, or of about 1 ng/mL or lower, in vitro.

In some embodiments, the antibody, or antigen-binding fragment thereof, neutralizes the BA.1 variant of SARS-CoV-2 with an IC50 of about 20 ng/mL or lower, or about 15 ng/mL or lower, in vitro.

In some embodiments, the antibody, or antigen-binding fragment thereof, neutralizes the XBB.1.5 variant of SARS-CoV-2 with an IC50 of about 5 ng/mL or lower, or about 4 ng/mL or lower, in vitro.

In some embodiments, the antibody, or antigen-binding fragment thereof, neutralizes the XBB.1.16 variant of SARS-CoV-2 with an IC50 of about 5 ng/mL or lower, or about 4 ng/mL or lower, in vitro.

In some embodiments, the antibody, or antigen-binding fragment thereof, neutralizes the XBB.1.5.10 variant of SARS-CoV-2 with an IC50 of about 5 ng/mL or lower, or about 4 ng/mL or lower, in vitro.

In some embodiments, the antibody, or antigen-binding fragment thereof, neutralizes the XBB.1.5.1 variant of SARS-CoV-2 with an IC50 of about 5 ng/mL or lower, or about 4 ng/mL or lower, in vitro.

In some embodiments, the antibody, or antigen-binding fragment thereof, neutralizes the XBB.2.3 variant of SARS-CoV-2 with an IC50 of about 5 ng/mL or lower, or about 4 ng/mL or lower, in vitro.

In some embodiments, the antibody, or antigen-binding fragment thereof, neutralizes the FL.1.5.1 variant of SARS-CoV-2 with an IC50 of about 5 ng/mL or lower, or about 4 ng/mL or lower, or about 3 ng/mL or lower, in vitro.

In some embodiments, the antibody, or antigen-binding fragment thereof, neutralizes the XBB.1.5.CONV0817 variant of SARS-CoV-2 with an IC50 of about 10 ng/mL or lower, or about 9 ng/mL or lower, or about 8 ng/mL or lower, in vitro.

In some embodiments, the antibody, or antigen-binding fragment thereof, neutralizes the BA.2.86 variant of SARS-CoV-2 with an IC50 of about 5 ng/mL or lower, or about 4 ng/mL or lower, or about 3 ng/mL or lower, in vitro.

In some embodiments, the antibody, or antigen-binding fragment thereof, neutralizes the HV.1 variant of SARS-CoV-2 with an IC50 of about 5 ng/mL or lower, or about 4 ng/mL or lower, or about 3 ng/mL or lower, in vitro.

In some embodiments, the antibody, or antigen-binding fragment thereof, neutralizes the HK.3 variant of SARS-CoV-2 with an IC50 of about 5 ng/mL or lower, or about 4 ng/mL or lower, in vitro.

In some embodiments, the antibody, or antigen-binding fragment thereof, neutralizes the JN.1 variant of SARS-CoV-2 with an IC50 of about 5 ng/mL or lower, or about 4 ng/mL or lower, in vitro.

In some embodiments, the antibody, or antigen-binding fragment thereof, neutralizes the JD.1.1 variant of SARS-CoV-2 with an IC50 of about 5 ng/mL or lower, or about 4 ng/mL or lower, in vitro.

In some embodiments, the antibody, or antigen-binding fragment thereof, neutralizes the BA.2 variant of SARS-CoV-2 with an IC50 of about 5 ng/mL or lower, or about 4 ng/mL or lower, or about 3 ng/mL or lower, or about 2 ng/mL or lower, in vitro.

In some embodiments, the antibody, or antigen-binding fragment thereof, neutralizes the GE.1.2.1 variant of SARS-CoV-2 with an IC50 of about 5 ng/mL or lower, or about 4 ng/mL or lower, or about 3 ng/mL or lower, in vitro.

In some embodiments, the antibody, or antigen-binding fragment thereof, neutralizes the JN.1.13.1 variant of SARS-CoV-2 with an IC50 of about 10 ng/mL or lower, or about 9 ng/mL or lower, in vitro.

In some embodiments, the antibody, or antigen-binding fragment thereof, neutralizes the KQ.1 variant of SARS-CoV-2 with an IC50 of about 5 ng/mL or lower, in vitro.

In some embodiments, the antibody, or antigen-binding fragment thereof, neutralizes the KP.1.1 variant of SARS-CoV-2 with an IC50 of about 10 ng/mL or lower, or about 9 ng/mL or lower, or about 8 ng/mL or lower, or about 7 ng/mL or lower, or about 6 ng/mL or lower, or about 5 ng/mL or lower, in vitro.

In some embodiments, the antibody, or antigen-binding fragment thereof, neutralizes the KP.3 variant of SARS-CoV-2 with an IC50 of about 25 ng/mL or lower, or about 20 ng/mL or lower, or about 15 ng/mL or lower, or about 14 ng/mL or lower, about 13 ng/mL or lower, or about 12 ng/mL or lower, in vitro.

In some embodiments, the antibody, or antigen-binding fragment thereof, neutralizes the KP.2 variant of SARS-CoV-2 with an IC50 of about 20 ng/mL or lower, or about 15 ng/mL or lower, or about 10 ng/mL or lower, in vitro.

In some embodiments, the antibody, or antigen-binding fragment thereof, neutralizes the JN.1.50 variant of SARS-CoV-2 with an IC50 of about 5 ng/mL or lower, or about 4 ng/mL or lower, or about 3 ng/mL or lower, in vitro.

In some embodiments, the antibody, or antigen-binding fragment thereof, neutralizes the KP.3.1.1 variant of SARS-CoV-2 with an IC50 of about 50 ng/mL or lower, or about 45 ng/mL or lower, or about 40 ng/mL or lower, or about 30 ng/mL or lower, or about 20 ng/mL or lower, in vitro.

In some embodiments, the antibody, or antigen-binding fragment thereof, neutralizes the LB.1 variant of SARS-CoV-2 with an IC50 of about 25 ng/mL or lower, or about 20 ng/mL or lower, or about 15 ng/mL or lower, in vitro.

In some embodiments, the antibody, or antigen-binding fragment thereof, neutralizes the XEC variant of SARS-CoV-2 with an IC50 of about 20 ng/mL or lower, or about 18 ng/mL or lower, or about 16 ng/mL or lower, or about 15 ng/mL or lower, or about 14 ng/mL or lower, in vitro.

In some embodiments, the antibody, or antigen-binding fragment thereof, neutralizes the MV.1 variant of SARS-CoV-2 with an IC50 of about 10 ng/mL or lower, or about 8 ng/mL or lower, or about 6 ng/mL or lower, or about 5 ng/mL or lower, in vitro.

In some embodiments, the antibody, or antigen-binding fragment thereof, neutralizes the LP.8.1 variant of SARS-CoV-2 with an IC50 of about 30 ng/mL or lower, about 25 ng/mL or lower, or about 20 ng/mL or lower, in vitro.

In some embodiments, the antibody, or antigen-binding fragment thereof, neutralizes the MC.10.2 variant of SARS-CoV-2 with an IC50 of about 100 ng/mL or lower, about 80 ng/mL or lower, about 70 ng/mL or lower, or about 65 ng/mL or lower, in vitro.

In some embodiments, the antibody, or antigen-binding fragment thereof, neutralizes the LF.7 variant of SARS-CoV-2 with an IC50 of about 20 ng/mL or lower, or about 18 ng/mL or lower, or about 16 ng/mL or lower, or about 14 ng/mL or lower, or about 12 ng/mL or lower, in vitro.

In some embodiments, the antibody, or antigen-binding fragment thereof, neutralizes the LP.8.1.2 variant of SARS-CoV-2 with an IC50 of about 30 ng/mL or lower, about 25 ng/mL or lower, about 20 ng/mL or lower, about 15 ng/mL or lower, about 14 ng/mL or lower, or about 13 ng/mL or lower, in vitro.

In some embodiments, the antibody, or antigen-binding fragment thereof, neutralizes the LF.7.2.1 variant of SARS-CoV-2 with an IC50 of about 20 ng/mL or lower, about 15 ng/mL or lower, about 10 ng/mL or lower, about 8 ng/mL or lower, about 6 ng/mL or lower, or about 6 ng/mL or lower, in vitro.

In some embodiments, the antibody, or antigen-binding fragment thereof, have a Tₘ of at least about 62-70° C., about 63-70° C., or about 64-70° C. In some embodiments, the antibody, or antigen-binding fragment thereof, have a Tₘ of at least about 62° C., about 63° C., about 64° C., about 65° C., about 66° C., about 67° C., about 68° C., about 69° C., or about 70° C.

In some embodiments, the antibody, or antigen-binding fragment thereof, have a Tₒₙ of at least about 56-70° C., about 57-70° C., or about 58-70° C. In some embodiments, the antibody, or antigen-binding fragment thereof, have a Tₒₙ of at least about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C.

In some embodiments, the antibody, or antigen-binding fragment thereof, has a serum half-life of about 40 days, about 50 days, about 60 days, about 70 days, about 80 days, about 90 days, about 100 days, about 110 days, about 120 days, about 130 days, about 140 days, about 150 days, about 160 days, about 170 days, about 180 days, about 190 days, or about 200 days.

In some embodiments, the antibody, or antigen-binding fragment thereof, has a serum half-life of about 40-200 days, about 45-200 days, about 50-160 days, about 50-140 days, about 40-130 days, about 45-130 days, about 50-100 days, about 60-100 days, about 60-90 days, about 50-90 days, about 50-80 days, about 40-70 days, about 60-80 days, about 55-75 days, about 65-95 days, about 70-90 days, about 90-100 days, about 70-80 days, about 80-130 days, about 100-150 days, about 110-140 days, about 120-140 days, about 120-160 days, about 130-170 days, about 140-180 days, about 150-190 days, or about 160-200 days.

In some embodiments, the antibody, or antigen-binding fragment thereof, is a human, humanized, primatized or chimeric antibody, or antigen-binding fragment thereof.

In some embodiments, the antibody, or antigen-binding fragment thereof, is bispecific or multispecific. In some embodiments, the bispecific or multispecific antibody, or antigen-binding fragment thereof, comprises at least one first antigen-binding domain (“ABD”) and at least one second ABD, wherein: (i) said first ABD comprises the VH CDR1, the VH CDR2, the VH CDR3, the VL CDR1, the VL CDR2, and the VL CDR3 of a first antibody; and (ii) said second ABD comprises the VH CDR1, the VH CDR2, the VH CDR3, the VL CDR1, the VL CDR2, and the VL CDR3 of a second antibody, wherein the first anti-CoV-S antibody is same as the second anti-CoV-S antibody, or wherein the first anti-CoV-S antibody is different from the second anti-CoV-S antibody.

In some embodiments, the first anti-CoV-S antibody binds to a first CoV-S, and the second anti-CoV-S antibody binds to a second CoV-S.

In some embodiments, the first anti-CoV-S antibody and the second anti-CoV-S antibody bind to: (i) the same coronavirus species, optionally wherein the first CoV-S and the second CoV-S are (a) both of SARS-CoV or (b) both of SARS-CoV-2, and optionally wherein the first anti-CoV-S antibody and the second anti-CoV-S antibody bind to the same or different epitopes on a CoV-S expressed by said SARS-CoV or SARS-CoV-2; or (ii) different coronavirus species, optionally wherein the first CoV-S and the second CoV-S are of (a) of SARS-CoV and of SARS-CoV-2, respectively, or (b) of SARS-CoV-2 and of SARS-CoV, respectively.

In some embodiments, the bispecific or multispecific antibody, or antigen-binding fragment thereof, comprises at least one first antigen-binding domain (“ABD”) and at least one second ABD, wherein: wherein: (a) said first ABD comprises the VH CDR1, the VH CDR2, the VH CDR3, the VL CDR1, the VL CDR2, and the VL CDR3 of a first anti-CoV-S antibody; and (b) said second ABD binds to an antigen which is not a CoV-S, optionally wherein said antigen is a cytokine, a cytokine receptor, or an immunomodulatory polypeptide.

In some embodiments, the antibody, or antigen-binding fragment thereof, comprises a Fab, Fab2, or scFv. In some embodiments, the antibody, or antigen-binding fragment thereof, comprises a constant region, an Fc region, or at least one domain thereof.

In some embodiments, the constant region or Fc region comprises a mutation which impairs at least one effector function, optionally FcR binding, complement binding, glycosylation, complement-dependent cytotoxicity (“CDC”), or antibody-dependent cellular cytotoxicity (“ADCC”).

In some embodiments, the constant or Fc region is human. In some embodiments, the human constant or Fc region is selected from a human IgG1, IgG2, IgG3 or IgG4 constant or Fc region. In some embodiments, said human constant or Fc region is a human IgG1 constant or Fc region. In some embodiments, the human constant or Fc region is a human IgG1 constant or Fc region, the VH comprises (e.g. consists) of SEQ ID NO: 19 and the VL comprises (e.g. consists) of SEQ ID NO: 29.

In one embodiment, the antibody, or antigen-binding fragment thereof, is a neutralizing antibody. In one embodiment, the antibody, or antigen-binding fragment thereof, is an affinity optimized antibody, or antigen-binding fragment thereof.

In one aspect, the present disclosure provides a nucleic acid molecule, e.g., a DNA or an mRNA molecule, encoding an antibody or antigen binding fragment thereof, as disclosed herein.

In another aspect, the present disclosure provides an isolated antibody, or antigen-binding fragment thereof, which competes for binding with the isolated antibody, or antigen-binding fragment thereof, as described herein.

In some embodiments, binding competition is measured using an assay selected from the group consisting of surface plasmon resonance (SPR), ELISAs, radioimmunoassays, Western blotting, and bio-layer interferometry (BLI). In one embodiment, KD is measured by SPR. In another embodiment, KD is measured by ELISA. In one embodiment, KD is measured by BLI.

In one aspect, the present disclosure provides an isolated antibody, or antigen-binding fragment thereof, which binds the same epitope as the isolated antibody, or antigen-binding fragment thereof, as described herein.

In some embodiments, epitope mapping is determined using an assay selected from the group consisting of surface plasmon resonance (SPR), ELISAs, radioimmunoassays, Western blotting, and bio-layer interferometry (BLI). In one embodiment, KD is measured by SPR. In another embodiment, KD is measured by ELISA. In one embodiment, KD is measured by BLI.

In another aspect, the present disclosure provides an affinity matured variant of any one of the isolated antibodies, or antigen-binding fragments thereof, as described herein.

In one aspect, the present disclosure provides a chimeric antigen receptor (“CAR”) comprising at least one antibody, or antigen-binding fragment thereof, as described herein.

In one aspect, the present disclosure provides an antibody drug conjugate (“ADC”) comprising: (a) at least one antibody, or antigen-binding fragment thereof, as described herein; and (b) a drug.

In some embodiments, the drug is: (i) an antiviral drug, optionally, remdesivir, favipiravir, darunavir, nelfinavir, saquinavir, lopinavir, nirmatrelvir, or ritonavir; (ii) an antihelminth drug, optionally ivermectin; (iii) an antiparasite drug, optionally hydroxychloroquine, chloroquine, or atovaquone; (iv) antibacterial vaccine, optionally the tuberculosis vaccine BCG; or (v) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (vi) an antihistamine drug, optionally bepotastine; (vii) an ACE inhibitor, optionally moexipril; (viii) a drug that inhibits priming of CoV-S, optionally a serine protease inhibitor, further optionally nafamostat; or (ix) a cytotoxic drug, optionally daunorubicin, mitoxantrone, doxorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, or epothilone.

In one aspect, the present disclosure provides a composition comprising at least one antibody, or antigen-binding fragment thereof, a CAR or an ADC as described herein. In one embodiment, the composition further comprises an additional anti-SARS-COV-S antibody, or antigen-binding fragment thereof.

In one aspect, the present disclosure provides a pharmaceutical composition comprising at least one antibody, or antigen-binding fragment thereof, a CAR or an ADC as described herein; and a pharmaceutically acceptable carrier or excipient. In one embodiment, the pharmaceutical composition further comprises an additional anti-SARS-COV-S antibody, or antigen-binding fragment thereof.

In one aspect, the present disclosure provides a method of treating infection by SARS-CoV, SARS-CoV-2, and/or another coronavirus optionally selected from the group consisting of MERS-CoV, HCoV-HKU1, HCoV-OC43, HCoV-229E, and HCoV-NL63, or treating a condition, symptom, disease, or disorder associated with said infection in a subject in need thereof, comprising administering to the subject a therapeutically effective amount of an antibody, or antigen-binding fragment thereof, a CAR or an ADC as described herein. In some embodiments, the antibody, or antigen-binding fragment thereof, the CAR or the ADC used in the method of treating the infection is administered intravenously. In some embodiments, the infection is SARS-CoV-2 and the method comprises administering to the subject a therapeutically effective amount of an antibody, or antigen-binding fragment thereof, as described herein (e.g. VYD2311) intravenously.

In some embodiments, the condition, symptom, disease, or disorder comprises at least one of bronchitis, pneumonia, respiratory failure, acute respiratory failure, organ failure, multi-organ system failure, pediatric inflammatory multisystem syndrome, acute respiratory distress syndrome, blood clot, a cardiac condition, myocardial injury, myocarditis, heart failure, cardiac arrest, acute myocardial infarction, dysrhythmia, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, a gastrointestinal complication, aspergillosis, increased susceptibility to infection with another virus or bacteria, and/or a pregnancy-related complication. In some embodiments, the infection is a chronic infection. In some embodiments, the infection is a chronic SARS-CoV-2 infection. In some embodiments, the infection is a long COVID.

In one aspect, the present disclosure provides a method of preventing infection by SARS-CoV, SARS-CoV-2, and/or another coronavirus optionally selected from the group consisting of MERS-CoV, HCoV-HKU1, HCoV-OC43, HCoV-229E, and HCoV-NL63 in a subject in need thereof, comprising administering to the subject a prophylactically effective amount of an antibody, or antigen-binding fragment thereof, a CAR or an ADC as described herein. In some embodiments, the antibody, or antigen-binding fragment thereof, the CAR or the ADC used in the method of preventing the infection is administered intramuscularly or subcutaneously. In some embodiments, the antibody, or antigen-binding fragment thereof, the CAR or the ADC is administered intramuscularly. In some embodiments, the infection is SARS-CoV-2 and the method comprises administering to the subject a prophylactically effective amount of an antibody, or antigen-binding fragment thereof, as described herein (e.g. VYD2311) intramuscularly or subcutaneously. In some embodiments, the infection is SARS-CoV-2 and the method comprises administering to the subject a prophylactically effective amount of an antibody, or antigen-binding fragment thereof, as described herein (e.g. VYD2311) intramuscularly.

In one aspect, the present disclosure provides a method of inducing an immune response against SARS-CoV, SARS-CoV-2, and/or another coronavirus optionally selected from the group consisting of MERS-CoV, HCoV-HKU1, HCoV-OC43, HCoV-229E, and HCoV-NL63 in a subject in need thereof, comprising administering at least one antibody, or antigen-binding fragment thereof, a CAR or an ADC as described herein. In some embodiments, the antibody, or antigen-binding fragment thereof, the CAR or the ADC as described herein is administered intramuscularly, intravenously or subcutaneously.

In some embodiments, the immune response elicits immunoprotection against SARS-CoV, SARS-CoV-2 and/or another coronavirus. In some embodiments, the immune response elicits immunoprotection against SARS-CoV-2 and the method comprises administering an antibody, or antigen-binding fragment thereof, as described herein (e.g. VYD2311) intramuscularly or subcutaneously. In some embodiments, the immune response elicits immunoprotection against SARS-CoV-2 and the method comprises administering an antibody, or antigen-binding fragment thereof, as described herein (e.g. VYD2311) intramuscularly.

In one aspect, the present disclosure provides a method of inhibiting or blocking infection of susceptible cells by SARS-CoV, SARS-CoV-2, and/or another coronavirus optionally selected from the group consisting of MERS-CoV, HCoV-HKU1, HCoV-OC43, HCoV-229E, and HCoV-NL63 in a subject in need thereof, comprising administering at least one antibody, or antigen-binding fragment thereof, a CAR or an ADC as described herein.

In one aspect, provided herein is a method of preventing the need for a subject infected with SARS-CoV, SARS-CoV-2, and/or another coronavirus optionally selected from the group consisting of MERS-CoV, HCoV-HKU1, HCoV-OC43, HCoV-229E, and HCoV-NL63 to be placed on a ventilator, or reducing the time that a subject infected with SARS-CoV or SARS-CoV-2 or another coronavirus optionally selected from the group consisting of MERS-CoV, HCoV-HKU1, HCoV-OC43, HCoV-229E, and HCoV-NL63 is on a ventilator, comprising administering to the subject a prophylactically or therapeutically effective amount of an antibody, or antigen-binding fragment thereof, a CAR or an ADC as described herein.

In one aspect, provided herein is a method of preventing the onset of pneumonia in a subject infected SARS-CoV, SARS-CoV-2, and/or another coronavirus optionally selected from the group consisting of MERS-CoV, HCoV-HKU1, HCoV-OC43, HCoV-229E, and HCoV-NL63, or treating pneumonia and/or the symptoms of pneumonia in a subject for a subject infected SARS-CoV or SARS-CoV-2 or another coronavirus optionally selected from the group consisting of MERS-CoV, HCoV-HKU1, HCoV-OC43, HCoV-229E, and HCoV-NL63, comprising administering to the subject a prophylactically or therapeutically effective amount of an antibody, or antigen-binding fragment thereof, a CAR or an ADC as described herein.

Where the antibody, or antigen-binding fragment thereof, as described herein, is administered intravenously, intramuscularly, or subcutaneously in the methods of the present disclosure, it is, in some embodiments, administered in a corresponding dose and/or interval for intravenous, intramuscular, or subcutaneous administration, as described herein.

In some embodiments, the infection is a chronic infection. In some embodiments, the infection is a chronic SARS-CoV-2 infection. In some embodiments, the infection is a long COVID.

In some embodiments, the subject is a human subject.

In some embodiments, the subject is an adult. In some embodiment is an adolescent, e.g., at least 12 years of age or older and weighting at least 40 kg.

In some embodiments, the subject is immunocompetent.

In some embodiments, the subject is immunocompromised.

In some embodiments, the subject is at risk of exposure to SARS-CoV, SARS-CoV-2, and/or another coronavirus.

In some embodiments, the subject has at least one risk factor which renders them more prone to a poor clinical outcome and/or progression to severe infection. In some embodiments, the at least one risk factor is one or more of (i) an old age such as over 55, 60 or 65 years old, (ii) diabetes, (iii) a chronic respiratory condition such as asthma, bronchiectasis, cystic fibrosis, another fibrotic condition, chronic obstructive pulmonary disease (COPD), damaged or scarred lung tissue, pulmonary embolism or pulmonary hypertension, (iv) obesity, (iv) hypertension, (v) a cardiac or cardiovascular condition, such as heart defects or abnormalities, heart failure, coronary artery disease, cardiomyopathies, or hypertension, (vi) a chronic inflammatory or autoimmune condition such as lupus and multiple sclerosis, (vii) an immunocompromised status which may be caused by cancer, undergoing chemotherapy, smoking, bone marrow or organ transplantation, immune deficiencies, poorly controlled HIV infection or AIDS, or prolonged use of corticosteroids or other immunosuppressive medications, (viii) cerebrovascular disease or stroke, (ix) chronic kidney disease, (x) chronic liver disease, such as alcohol-related liver disease, non-alcoholic fatty liver disease, autoimmune hepatitis, or cirrhosis, (xi) Down syndrome, (xii) hemoglobin blood disorders, such as sickle cell disease and thalassemia, and (xiii) neurologic conditions such as dementia or Parkinson's disease.

In some embodiments, the subject is not responsive or ineligible for treatment with existing and/or approved therapeutics for treating COVID.

In some embodiments, the antibody, or antigen-binding fragment thereof, is administered in combination with one or more existing and/or approved therapeutics for treating COVID. In one embodiment, the administration is at the same time. In one embodiment, the administration is sequential. In some embodiments, the antibody, or antigen-binding fragment thereof, is administered in combination with Paxlovid, Remdesivir, Molnupiravir, or any combinations thereof. In some embodiments, the combination treatment regimens with existing and/or approved COVID therapeutics, e.g., Paxlovid, reduces the risk of COVID rebound, i.e., the recurrence of COVID symptoms.

In some embodiments, the antibody, or antigen-binding fragment thereof, is administered in combination with an additional at least one anti-CoV-S antibody or antigen-binding fragment. In one embodiment, the administration is at the same time. In one embodiment, the administration is sequential. In some embodiments, the additional anti-CoV-S antibody or antigen-binding fragment is ADG20. In some embodiments, the additional anti-CoV-S antibody or antigen-binding fragment is VYD222.

In one embodiment, the antibody, or antigen-binding fragment thereof, is administered as a single injection. In one embodiment, the antibody, or antigen-binding fragment thereof, is administered at multiple injection sites to deliver a single dose.

In some embodiments, the antibody, or antigen-binding fragment thereof, is administered intravenously. In one embodiment, the antibody, or antigen-binding fragment thereof, is administered via an intravenous (IV) push. In another embodiment, the antibody, or antigen-binding fragment thereof, is administered via an IV bolus. In another embodiment, the antibody, or antigen-binding fragment thereof, is administered via IV infusion. In other embodiments, the antibody, or antigen-binding fragment thereof, is administered intramuscularly. In other embodiments, the antibody, or antigen-binding fragment thereof, is administered subcutaneously.

In some embodiments, the antibody, or antigen-binding fragment thereof, is administered at a dose of about 100 mg to 9000 mg, about 100 mg to 8500 mg, about 100 to 8000 mg, about 100 mg to 7500 mg, about 100 to 7000 mg, about 100 mg to 6500 mg, about 100 to 6000 mg, about 100 mg to 5500 mg, about 100 mg to 5000 mg, about 100 mg to 4500 mg, about 100 mg to 4000 mg, about 100 mg to about 3500 mg, about 100 mg to about 3000 mg, about 100 mg to about 2500 mg, about 100 mg to about 2000 mg, about 200 mg to about 1500 mg, about 300 mg to about 600 mg, about 500 mg to about 1200 mg, about 300 mg to about 1200 mg, about 500 to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg, about 4500 mg to about 5000 mg, about 5000 mg to about 5500 mg, about 5500 mg to about 6000 mg, about 6500 mg to about 7000 mg, about 7500 mg to about 8000 mg, about 8000 mg to about 8500 mg, or about 8500 mg to about 9000 mg.

In some embodiments, the antibody, or antigen-binding fragment thereof, is administered at a dose of about 100 mg, about 200 mg, about 300 mg, about 400 mg, about 450 mg, about 500 mg, about 600 mg, about 700 mg, about 800 mg, about 900 mg, about 1000 mg about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, about 6000 mg, about 6100 mg, about 6200 mg, about 6300 mg, about 6400 mg, about 6500 mg, about 6600 mg, about 6700 mg, about 6800 mg, about 6900 mg, about 7000 mg, about 7100 mg, about 7200 mg, about 7300 mg, about 7400 mg, about 7500 mg, about 7600 mg, about 7700 mg, about 7800 mg, about 7900 mg, about 8000 mg, about 8100 mg, about 8200 mg, about 8300 mg, about 8400 mg, about 8500 mg, about 8600 mg, about 8700 mg, about 8800 mg, about 8900 mg, or about 9000 mg.

In some embodiments, the antibody, or antigen-binding fragment thereof, is administered at a dose of about 1000 mg intramuscularly. In some embodiments, the antibody, or antigen-binding fragment thereof, is administered at a dose of about 2000 mg intravenously. In some embodiments, the antibody, or antigen-binding fragment thereof, is administered at a dose of about 4500 mg intravenously. In some embodiments, the antibody, or antigen-binding fragment thereof, is administered at a dose of about 1250 mg subcutaneously.

In some embodiments, the antibody, or antigen-binding fragment thereof, is administered at a dose of about 250 mg to about 4500 mg.

In some embodiments, the antibody, or antigen-binding fragment thereof, is administered at a dose of about 250 mg to about 1000 mg intramuscularly. In some embodiments, the antibody, or antigen-binding fragment thereof, is administered at a dose of about 250 mg to about 1250 mg subcutaneously. In some embodiments, the antibody, or antigen-binding fragment thereof, is administered at a dose of about 250 mg to about 750 mg subcutaneously. In some embodiments, the antibody, or antigen-binding fragment thereof, is administered at a dose of about 250 mg to about 4500 mg intravenously. In some embodiments, the antibody, or antigen-binding fragment thereof, is administered at a dose of about 1500 mg to about 4500 mg intravenously. In some embodiments, the antibody, or antigen-binding fragment thereof, is administered at a dose of about 1500 mg to about 2000 mg intravenously. In some embodiments, the antibody, or antigen-binding fragment thereof, is administered at a dose of about 2000 mg to about 4500 mg intravenously.

In some embodiments, the antibody, or antigen-binding fragment thereof, is administered at a dose of about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg intramuscularly.

In some embodiments, the antibody, or antigen-binding fragment thereof, is administered at a dose of about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, or about 1250 mg subcutaneously.

In some embodiments, the antibody, or antigen-binding fragment thereof, is administered at a dose of about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, or about 4500 mg intravenously.

In one embodiment, the antibody, or antigen-binding fragment thereof, is administered once. In one embodiment, the antibody, or antigen-binding fragment thereof, is administered twice. In one embodiment, the antibody, or antigen-binding fragment thereof, is administered weekly. In another embodiment, the antibody, or antigen-binding fragment thereof, is administered daily, weekly, every two weeks, monthly, or every two months. In another embodiment, the antibody, or antigen-binding fragment thereof, is administered every three months, every six months, or every year. In one embodiment, the antibody, or antigen-binding fragment thereof, is administered once every three months. In another embodiment, the antibody, or antigen-binding fragment thereof, is administered once every six months. In yet another embodiment, the antibody, or antigen-binding fragment thereof, is administered once every year. In some embodiments, the antibody, or antigen-binding fragment thereof, is administered intramuscularly every three months. In other embodiments, the antibody, or antigen-binding fragment thereof, is administered subcutaneously every three months.

In some embodiments, the antibody, or antigen-binding fragment thereof, is administered at a loading dose of about 100 mg to about 4500 mg. In some embodiments, the antibody, or antigen-binding fragment thereof, is administered at a loading dose of about 100 mg to about 4500 mg, followed by at least one maintenance dose of about 100 mg to about 4500 mg.

In some embodiments, the antibody, or antigen-binding fragment thereof, is administered via self-administration. In some embodiments, the antibody, or antigen-binding fragment thereof, is administered by a healthcare provider. In some embodiments, the antibody, or antigen-binding fragment thereof, is administered subcutaneously via self-administration.

In one embodiment, the antibody, or antigen-binding fragment thereof, is administered weekly for about four weeks, once weekly for about a month, weekly for about 5 weeks, weekly for about 6 weeks, weekly for about 7 weeks, weekly for about two months, or weekly for about three months.

In one embodiment, the antibody, or antigen-binding fragment thereof, is administered monthly for about one month, monthly for about two months, monthly for about three months, or monthly for about four months. In one embodiment, the antibody, or antigen-binding fragment thereof, is administered once, or multiple times, e.g., twice, three times, four times, or five times, for the first one month, the first two months, the first three months, the first four months, or the first five months.

In one embodiment, the antibody, or antigen-binding fragment thereof, is administered to a recipient subject with a frequency of once every twenty-six weeks or less, such as once every sixteen weeks or less, once every eight weeks or less, once every four weeks or less, once every two weeks or less, once every week or less, or once daily or less.

The antibody, or antigen-binding fragment thereof, may be administered every year or less, every 6 months or less, every three months or less, every one month or less, every two weeks or less, every week or less, once daily or less, multiple times per day, and/or every few hours. In one embodiment, the administration is given every month, every 2 months, every 3 months, every 4 months, every 5 months, every 6 months, every 7 months, every eight months, every 9 months, every 10 months, every 11 months, or once a year.

In some embodiments, the antibody, or antigen-binding fragment thereof, is administered in combination with one or more additional anti-CoV-S-antibodies.

In some embodiments, administering the antibody, or antigen-binding fragment thereof, results in about 30%, about 40%, about 50%, about 60%, or about 70% relative risk reduction, e.g., for at least 2 months, at least 4 months, at least 6 months, at least 8 months, at least 10 months, or at least 12 months.

In some embodiments, the methods provide an onset of protection against the coronavirus at about 1-20 days, about 1-10 days, about 3-10 days, about 3-7 days, about 5-20 days, or about 5-10 days after administration of the antibody, or antigen-binding fragment thereof.

In some embodiments, the methods provide an onset of protection against the coronavirus at about 3 days, about 4 days, about 5 days, about 6 days, about 7 days, about 8 days, about 9 days, about 10 days, about 11 days, about 12 days, about 13 days, about 14 days, or about 15 days after administration of the antibody, or antigen-binding fragment thereof.

In some embodiments, the methods reduce the duration of symptoms and/or the likelihood of hospitalization or death of the subject.

In one aspect, the present disclosure provides a method of producing the antibody, or antigen-binding portion thereof, as described herein, the method comprising expressing the antibody, or antigen-binding portion thereof, in a recombinant cell, and isolating the antibody, or antigen-binding portion thereof, from the cell. In some embodiments, the method further comprises formulating the antibody, or antigen-binding portion thereof, isolated from the cell into a pharmaceutical composition.

In another aspect, the present disclosure provides an isolated nucleic acid molecule encoding the antibody, or antigen-binding fragment thereof, of the present disclosure, as described herein.

In yet another aspect, the present disclosure provides an isolated mRNA molecule encoding the antibody, or antigen-binding fragment thereof, of the disclosure, as described herein.

In one aspect, the present disclosure provides a composition comprising the isolated nucleic acid molecule, or the isolated mRNA molecule of the disclosure, as described herein.

In another aspect, the present disclosure provides a kit comprising the antibody, or antigen-binding fragment thereof, the isolated nucleic acid molecule, or the isolated mRNA molecule, of the disclosure, as described herein, and instructions for use.

In yet another aspect, the present disclosure provides a vial comprising the antibody, or antigen-binding fragment thereof, the isolated nucleic acid molecule, or the isolated mRNA molecule, of the disclosure, as described herein.

In some embodiments, the vial is about 1 mL, about 2 mL, about 4 mL, about 8 mL, about 12 mL, about 16 mL, about 20 mL, or about 24 mL in volume.

In some embodiments, each vial comprises about 100 mg, about 200 mg, about 300 mg, about 500 mg, about 600 mg, about 700 mg, about 800 mg, about 900 mg, about 1000 mg, about 1500 mg, about 2000 mg, about 2500 mg, about 4500 mg, about 5000 mg, about 5500 mg, about 6000 mg, about 6500 mg, about 7000 mg, about 7500 mg, about 8000 mg, about 8500 mg, or about 9000 mg, of the antibody or antigen-binding fragment thereof.

In yet another aspect, the present disclosure provides a unit dose form (e.g. a vial or pre-filled injection delivery device) comprising the antibody, or antigen-binding fragment thereof, the isolated nucleic acid molecule, or the isolated mRNA molecule, of the disclosure, as described herein.

In some embodiments, a unit dose form or vial, as described herein, contains a unit dose of about 250 mg to about 4500 mg (e.g. about 250 mg to about 1250 mg) of the antibody, or antigen-binding fragment thereof.

In some embodiments, the unit dose form or vial contains a unit dose of about 1000 mg of the antibody, or antigen-binding fragment thereof, and may be suitable for intramuscular administration. In some embodiments, the unit dose form or vial contains a unit dose of about 1250 mg of the antibody, or antigen-binding fragment thereof, and may be suitable for subcutaneous administration. In some embodiments, the unit dose form or vial contains a unit dose of about 2000 mg of the antibody, or antigen-binding fragment thereof, and may be suitable for intravenous administration. In some embodiments, the unit dose form or vial contains a unit dose of about 4500 mg of the antibody, or antigen-binding fragment thereof, and may be suitable for intravenous administration.

In some embodiments, the unit dose form or vial contains a unit dose of about 250 mg to about 1000 mg of the antibody, or antigen-binding fragment thereof, and may be suitable for intramuscular administration. In some embodiments, the unit dose form or vial contains a unit dose of about 250 mg to about 1250 mg (e.g. about 250 mg to about 750 mg) of the antibody, or antigen-binding fragment thereof, and may be suitable for subcutaneous administration. In some embodiments, the unit dose form or vial contains a unit dose of about 250 mg to about 4500 mg (e.g. about 1500 mg to about 4500 mg, or more particularly about 1500 mg to about 2000 mg, or about 2000 mg to about 4500 mg) of the antibody, or antigen-binding fragment thereof, and may be suitable for intravenous administration.

In some embodiments, the unit dose form or vial contains a unit dose of about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of the antibody, or antigen-binding fragment thereof, and may be suitable for intramuscular administration.

In some embodiments, the unit dose form or vial contains a unit dose of about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, about 1050 mg, about 1100 mg, about 1150 mg, about 1200 mg, or about 1250 mg of the antibody, or antigen-binding fragment thereof, and may be suitable for subcutaneous administration.

In some embodiments, the unit dose form or vial contains a unit dose of about the antibody, or antigen-binding fragment thereof, is administered at a dose of about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, or about 4500 mg of the antibody, or antigen-binding fragment thereof, and may be suitable for intravenous administration.

### BRIEF DESCRIPTION OF DRAWINGS

FIG. 1 depicts the improved thermal stability for VYD2311 with higher T_{M} and Tₒₙ than the comparator antibody (control mAb).

FIG. 2 depicts the improved colloidal stability for VYD2311 with higher T_{scattering} and Tₒₙ than the comparator antibody (control mAb).

FIG. 3 depicts the comparison of observed serum concentration of VYD2311 in subjects following a single IM administration of 1000 mg VYD2311 and the modeled adintrevimab concentration at same timepoints.

FIG. 4 depicts the observed serum concentration of VYD2311 (mean±standard deviation) in subjects following a single IM administration of 1000 mg VYD2311, a single IV administration of 2000 mg VYD2311, a single IV administration of 4500 mg VYD2311, or a single SC administration of 1250 mg VYD2311, at the indicated timepoints.

FIG. 5 depicts the comparison of observed mean serum concentration of VYD2311 and the model-predicted control antibody concentration in subjects receiving the indicated doses and routes of administration.

FIG. 6 depicts the comparison of model-predicted geometric median titers (GMT) of VYD2311 and the control mAb dosed every 3 months (Q3M) or every 6 months (Q6M) via IV administration at the end of 6 months variants LP.8.1 and KP.3.1.1.

FIG. 7 depicts the model-predicted geometric median titers (GMT) of VYD2311 dosed every 3 months (Q3M, top panel) or every 6 months (Q6M, bottom panel) via IV, IM or SC administrations at the end of 6 months for variants LP.8.1 and KP.3.1.1.

FIG. 8 depicts the serum virus neutralizing antibody (sVNA) titer for VYD2311 (2000 mg IV, or 4500 IV), and other COVID-19 mAbs (adintrevimab, 300 mg IM; and REGEN-CoV (1200 mg IV)).

FIG. 9 depicts the improved thermal stability for ADI-90031 (31), ADI-90032 (32), ADI-90033 (33) and VYD2311 with higher T_{M} and Tₒₙ than the comparator antibody (control mAb).

### DETAILED DESCRIPTION

The present disclosure is based on the surprising discovery of a new antibody with multiple improved properties, e.g., improved in vitro neutralization efficacy across all SARS-CoV-2 variants of concern (VOCs) described to date, higher binding affinity, and more favorable thermal and colloidal stability, as compared to other existing antibodies to date. Indeed, the new antibody is notably more advantageous as its improved thermal and colloidal stability in vitro can lead to a more stable product at higher concentrations and higher temperatures, an improved shelf life and half-life, and also allows for smaller dosing volumes. This antibody represents a more promising candidate for therapeutic development and provides a framework for the development of vaccines that induce broadly neutralizing antibody responses.

### A. Definitions

It is to be understood that this invention is not limited to the particular methodology, protocols, cell lines, animal species or genera, and reagents described, as such may vary. It is also to be understood that the terminology used herein is for the purpose of describing particular embodiments only and is not intended to limit the scope of the present invention, which will be limited only by the appended claims. As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

The term “about” in relation to a numerical value x is optional and means, for example, x+5%.

Spike protein (S protein): As used herein, unless stated otherwise S protein includes any coronavirus form of S protein. The term coronavirus S protein (“CoV-S”) is used to describe the S protein of any coronaviruses. In particular, the “SARS-CoV-S” and “SARS-CoV-2-S” encompass the following S protein of SARS-CoV and of SARS-CoV-2 amino acid sequences:

SARS-COV-S:
(SEQ ID NO: 1)
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFLMDLEGKQGNFK
NLREFVFKNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTVEKGIYQ
TSNFRVQPTESIVRFPNITNLCPFGEVENATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQSYGFQPTNGV
GYQPYRVVVLSFELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDVNCTEVPVAIHADQ
LTPTWRVYSTGSNVFQTRAGCLIGAEHVNNSYECDIPIGAGICASYQTQTNSPGSASSVASQSI
IAYTMSLGAENSVAYSNNSIAIPTNFTISVTTEILPVSMTKTSVDCTMYICGDSTECSNLLLQY
GSFCTQLNRALTGIAVEQDKNTQEVFAQVKQIYKTPPIKDFGGFNFSQILPDPSKPSKRSFIEDL
LFNKVTLADAGFIKQYGDCLGDIAARDLICAQKFNGLTVLPPLLTDEMIAQYTSALLAGTITS
GWTFGAGAALQIPFAMQMAYRFNGIGVTQNVLYENQKLIANQFNSAIGKIQDSLSSTASALG
KLQDVVNQNAQALNTLVKQLSSNFGAISSVLNDILSRLDPPEAEVQIDRLITGRLQSLQTYVT
QQLIRAAEIRASANLAATKMSECVLGQSKRVDFCGKGYHLMSFPQSAPHGVVFLHVTYVPA
QEKNFTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKNLNES
LIDLQELGKYEQGSGYIPEAPRDGQAYVRKDGEWVLLSTFLGRSLEVLFQGPGHHHHHHHHS
AWSHPQFEKGGGSGGGGSGGSAWSHPQFEK (1288 amino acids, encoded by
SEQ ID NO: 2),

but also any mutants, splice variants, isoforms, orthologs, homologs, and n s sequence.

SARS-COV-2-S:
(SEQ ID NO: 5)
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFLMDLEGKQGNFK
NLREFVFKNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTVEKGIYQ
TSNFRVQPTESIVRFPNITNLCPFGEVENATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQSYGFQPTNGV
GYQPYRVVVLSFELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDVNCTEVPVAIHADQ
LTPTWRVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSNLLLQY
GSFCTQLNRALTGIAVEQDKNTQEVFAQVKQIYKTPPIKDFGGFNFSQILPDPSKPSKRSFIEDL
LFNKVTLADAGFIKQYGDCLGDIAARDLICAQKFNGLTVLPPLLTDEMIAQYTSALLAGTITS
GWTFGAGAALQIPFAMQMAYRFNGIGVTQNVLYENQKLIANQFNSAIGKIQDSLSSTASALG
KLQDVVNQNAQALNTLVKQLSSNFGAISSVLNDILSRLDKVEAEVQIDRLITGRLQSLQTYVT
QQLIRAAEIRASANLAATKMSECVLGQSKRVDFCGKGYHLMSFPQSAPHGVVFLHVTYVPA
QEKNFTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKNLNES
LIDLQELGKYEQYIKWPWYIWLGFIAGLIAIVMVTIMLCCMTSCCSCLKGCCSCGSCCKFDED
DSEPVLKGVKLHYT, (1273 amino acids, encoded by SEQ ID NO: 6),

but also any mutants, splice variants, isoforms, orthologs, homologs, and variants of this sequence. In some embodiments, the CoV-S comprises a polypeptide sequence having at least 85%, 90%, 95%, 96%, 97%, 98%, or 99% identity to either SEQ ID NO: 1 or SEQ ID NO:5.

“Effective treatment or prevention of CoV infection” herein refers to eliminating CoV from the subject or preventing the expansion of CoV in the subject or eliminating or reducing the symptoms such as fever, cough, shortness of breath, runny nose, congestion, conjunctivitis, and/or gastrointestinal symptoms after administration of an effective amount of an anti-CoV-S antibody or antigen-binding fragment thereof according to the disclosure. In some instances, effective treatment may eliminate the need for the subject to be placed on a ventilator or reduce the time the subject needs to be on a ventilator. The treatment may be effected as a monotherapy or in association with another active agent such as an antiviral agent or anti-inflammatory agent by way of example.

As used herein, “treatment” is an approach for obtaining beneficial or desired clinical results. For purposes of this disclosure, beneficial or desired clinical results include, but are not limited to, one or more of the following: improvement in any aspect of COV-S-related conditions such as fever or cough. For example, in the context of CoV infection treatment this includes lessening severity, alleviation of fever, cough, shortness of breath, and other associated symptoms, reducing frequency of recurrence, increasing the quality of life of those suffering from the CoV-related symptoms, and decreasing dose of other medications required to treat the CoV-related symptoms. Other associated symptoms include, but are not limited to, diarrhea, conjunctivitis, loss of smell, and loss of taste. Still other symptoms which may be alleviated or prevented include inflammation, cytokine storm and/or sepsis.

“Reducing incidence” or “prophylaxis” or “prevention” means any of reducing severity for a particular disease, condition, symptom, or disorder (the terms disease, condition, and disorder are used interchangeably throughout the application). Reduction in severity includes reducing drugs and/or therapies generally used for the condition by, for example, reducing the need for, amount of, and/or exposure to drugs or therapies. Reduction in severity also includes reducing the duration, and/or frequency of the particular condition, symptom, or disorder (including, for example, delaying or increasing time to next episodic attack in an individual). This further includes eliminating the need for the subject to be placed on a ventilator or reducing the time the subject needs to be on a ventilator.

“Ameliorating” one or more symptoms of CoV infection-related conditions means a lessening or improvement of one or more symptoms of the condition, e.g., fever or cough or shortness of breath as compared to not administering an anti-CoV-S antagonist antibody. “Ameliorating” also includes shortening or reduction in duration of a symptom. Again, this may include eliminating the need for the subject to be placed on a ventilator or reducing the time the subject needs to be on a ventilator.

As used herein, “controlling CoV-related symptom” or “controlling” another CoV-S-related condition refers to maintaining or reducing severity or duration of one or more symptoms of the condition (as compared to the level before treatment). For example, the duration or severity or frequency of symptoms is reduced by at least about any of 10%, 20%, 30%, 40%, 50%, 60%, 70%, 80%, 90%, or 100% in the individual as compared to the level before treatment. The reduction in the duration or severity, or frequency of symptoms can last for any length of time, e.g., 1 day, 2, days, 3 days, 4 days, 5 days, 6 days, 1 week, 2 weeks, 4 weeks (1 month), 8 weeks (2 months), 16 weeks (3 months), 4 months, 5 months, 6 months, 9 months, 12 months, etc.

As used therein, “delaying” the development of a CoV-S-related condition such as shortness of breath, bronchitis, or pneumonia e.g., interstitial), means to defer, hinder, slow, retard, stabilize, and/or postpone progression of the condition or disease. This delay can be of varying lengths of time, depending on the history of the condition or disease and/or individuals being treated. As is evident to one skilled in the art, a sufficient or significant delay can, in effect, encompass prevention, in that the individual does not develop symptoms. A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Development” or “progression” of a CoV-related condition such as cough or fever means initial manifestations and/or ensuing progression of the disorder. Development of cough or fever can be detectable and assessed using standard clinical techniques as well known in the art. However, development also refers to progression that may be undetectable. For purpose of this disclosure, development, or progression refers to the biological course of the symptoms. “Development” includes occurrence, recurrence, and onset. As used herein “onset” or “occurrence” of a condition includes initial onset and/or recurrence.

As used herein, the term “immunocompromised” refers to a subject having significant immune compromise, defined as any of the following: actively treated for solid tumor or hematologic malignancies; hematologic malignancies associated with poor response to COVID-19 vaccines (e.g., acute leukemia, chronic lymphocytic leukemia, non-Hodgkin lymphoma, or multiple myeloma (regardless of treatment); solid organ transplant recipient or an islet transplant recipient taking immunosuppressive therapy; chimeric antigen receptor (CAR)-T-cell therapy or hematopoietic stem cell transplant (within 2 years of transplantation or taking immunosuppressive therapy); moderate or severe primary immunodeficiency (e.g., common variable immunodeficiency disease, severe combined immunodeficiency, DiGeorge syndrome, Wiskott-Aldrich syndrome; advanced HIV infection (CD4 cell count<200 cells/mm³, history of an AIDS-defining illness without immune reconstitution, or clinical manifestations of symptomatic HIV); or taking high-dose corticosteroids (≥20 mg of prednisone or equivalent per day when administered for at least 2 weeks), cancer chemotherapeutic agents classified as severely immunosuppressive, and biologic agents that are immunosuppressive or immunomodulatory (e.g., B-cell depleting agents (or within the past year)), alkylating agents, antimetabolites, transplant-related immunosuppressive drugs, TNF blockers, or other immunosuppressive or immunomodulatory biologic agents for rheumatic diseases.

As used herein, the phrase “at risk of exposure to SARS-CoV, SARS-CoV-2, and/or another coronavirus” refers to a subject at risk of acquiring SARS-CoV, SARS-CoV-2, and/or another coronavirus due to, for example, regular unmasked face-to-face interactions in indoor settings, e.g., work place, grocery store, gym facility, public transportation, etc.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological, and/or behavioral symptoms of the disease, its complications and intermediate pathological phenotypes presenting during development of the disease. For therapeutic use, beneficial or desired results include clinical results such as reducing symptom intensity, duration, or frequency, and decreasing one or more symptoms resulting from CoV infection, including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients, eliminating the need for the subject to be placed on a ventilator or reducing the time the subject needs to be on a ventilator.

An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “loading dose”, refers to an initial dose of a drug, compound, or pharmaceutical composition administered to a subject, and is followed by one or more maintenance dose(s) thereof. Generally, a single loading dose is administered, and multiple loading doses are contemplated herein. In some embodiments, the amount of loading dose administered exceeds the amount of the maintenance dose administered, so as to achieve the desired steady-state concentration of the drug, compound, or pharmaceutical composition earlier than can be achieved with the maintenance dose.

A “maintenance dose”, as used herein, refers to one or more doses of a drug, compound, or pharmaceutical composition administered to a subject over a treatment period. Usually, the maintenance doses are administered at spaced treatment intervals, such as approximately every week, every 2 weeks, every 3 weeks, every 4 weeks, every month, every 2 months, every 3 months, every 4 months, every 5 months, every 6 months, every 7 months, every 8 months, every 9 months, every 10 months, every 11 months, or every 12 months.

A “suitable host cell” or “host cell” generally includes any cell wherein the subject anti-CoV-S antibodies and antigen-binding fragments thereof can be produced recombinantly using techniques and materials readily available. For example, the anti-CoV-S antibodies and antigen-binding fragments thereof of the present disclosure can be produced in genetically engineered host cells according to conventional techniques. Suitable host cells are those cell types that can be transformed or transfected with exogenous DNA and grown in culture, and include bacteria, fungal cells (e.g., yeast), and cultured higher eukaryotic cells (including cultured cells of multicellular organisms), particularly cultured mammalian cells, e.g., human or non-human mammalian cells. In an exemplary embodiment these antibodies may be expressed in CHO cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor, N.Y.: Cold Spring Harbor Laboratory Press (1989), and Current Protocols in Molecular Biology, Ausubel et al., editors, New York, NY: Green and Wiley and Sons (1993).

In some exemplary embodiments the antibodies may be expressed in mating competent yeast, e.g., any haploid, diploid or tetraploid yeast that can be grown in culture. Yeast useful in fermentation expression methods may exist in a haploid, diploid, or other polyploid form.

A “selectable marker” herein refers to a gene or gene fragment that confers a growth phenotype (physical growth characteristic) on a cell receiving that gene as, for example through a transformation event. The selectable marker allows that cell to survive and grow in a selective growth medium under conditions in which cells that do not receive that selectable marker gene cannot grow. Selectable marker genes generally fall into several types, including positive selectable marker genes such as a gene that confers on a cell resistance to an antibiotic or other drug, temperature when two temperature sensitive (“ts”) mutants are crossed or a ts mutant is transformed; negative selectable marker genes such as a biosynthetic gene that confers on a cell the ability to grow in a medium without a specific nutrient needed by all cells that do not have that biosynthetic gene, or a mutagenized biosynthetic gene that confers on a cell inability to grow by cells that do not have the wild type gene; and the like.

An “expression vector” herein refers to DNA vectors containing elements that facilitate manipulation for the expression of a foreign protein within the target host cell, e.g., a bacterial, insect, yeast, plant, amphibian, reptile, avian, or mammalian cell, e.g., a CHO or HEK cell. Conveniently, manipulation of sequences and production of DNA for transformation may first performed in a bacterial host, e.g. E. coli, and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, (b) complement auxotrophic deficiencies, or (c) supply critical nutrients not available from complex media. Exemplary vectors and methods for transformation of yeast are described, for example, in Burke, D., Dawson, D., & Stearns, T., Methods in yeast genetics: a Cold Spring Harbor Laboratory course manual, Plainview, NY: Cold Spring Harbor Laboratory Press (2000). Expression vectors for use in the methods of the disclosure may include yeast or mammalian specific sequences, including a selectable auxotrophic or drug marker for identifying transformed host strains. A drug marker may further be used to amplify copy number of the vector in a yeast host cell.

The polypeptide coding sequence of interest is operably linked to transcriptional and translational regulatory sequences that provide for expression of the polypeptide in the desired host cells, e.g., yeast or mammalian cells. These vector components may include, but are not limited to, one or more of the following: an enhancer element, a promoter, and a transcription termination sequence. Sequences for the secretion of the polypeptide may also be included, e.g. a signal sequence, and the like. An origin of replication, e.g., a yeast or mammalian origin of replication, is optional, as expression vectors may be integrated into the host cell genome.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites or alternatively via a PCR/recombination method familiar to those skilled in the art (GATEWAY® Technology (universal method for cloning DNA); Invitrogen, Carlsbad California). If such sites do not exist, the synthetic oligonucleotide adapters or linkers are used in accordance with conventional practice.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (that increase levels of transcription in response to absence of a repressor). Inducible promoters may initiate increased levels of transcription from DNA under their control in response to some change in culture conditions, e.g., the presence or absence of a nutrient or a change in temperature.

The promoter fragment may also serve as the site for homologous recombination and integration of the expression vector into the same site in the host cell, e.g., yeast or mammalian cell, genome; alternatively, a selectable marker may be used as the site for homologous recombination. Suitable promoters for use in different eukaryotic and prokaryotic cells are well known and commercially available.

The polypeptides of interest may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the polypeptide coding sequence that is inserted into the vector. The heterologous signal sequence selected preferably is one that is recognized and processed through one of the standard pathways available within the host cell, e.g., a mammalian cell, an insect cell, or a yeast cell. Additionally, these signal peptide sequences may be engineered to provide for enhanced secretion in expression systems. Secretion signals of interest also include mammalian and yeast signal sequences, which may be heterologous to the protein being secreted, or may be a native sequence for the protein being secreted. Signal sequences include pre-peptide sequences, and in some instances may include propeptide sequences. Many such signal sequences are known in the art, including the signal sequences found on immunoglobulin chains, e.g., K28 preprotoxin sequence, PHA-E, FACE, human MCP-1, human serum albumin signal sequences, human Ig heavy chain, human Ig light chain, and the like. For example, see Hashimoto et. al., Protein Eng., 11(2):75 (1998); and Kobayashi et. al., Therapeutic Apheresis, 2(4):257 (1998)).

Transcription may be increased by inserting a transcriptional activator sequence into the vector. These activators are cis-acting elements of DNA, usually about from 10 to 300 bp, which act on a promoter to increase its transcription. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter.

Expression vectors used in eukaryotic host cells may also contain sequences necessary for the termination of transcription and for stabilizing the mRNA. Such sequences are commonly available from 3′ to the translation termination codon, in untranslated regions of eukaryotic or viral DNAs or cDNAs. These regions contain nucleotide segments transcribed as polyadenylated fragments in the untranslated portion of the mRNA.

Construction of suitable vectors containing one or more of the above-listed components employs standard ligation techniques or PCR/recombination methods. Isolated plasmids or DNA fragments are cleaved, tailored, and re-ligated in the form desired to generate the plasmids required or via recombination methods. For analysis to confirm correct sequences in plasmids constructed, the ligation mixtures are used to transform host cells, and successful transformants selected by antibiotic resistance (e.g. ampicillin or Zeocin) where appropriate. Plasmids from the transformants are prepared, analyzed by restriction endonuclease digestion, and/or sequenced.

As an alternative to restriction and ligation of fragments, recombination methods based on specific attachment (“att”) sites and recombination enzymes may be used to insert DNA sequences into a vector. Such methods are described, for example, by Landy, Ann. Rev. Biochem., 58:913-949 (1989); and are known to those of skill in the art. Such methods utilize intermolecular DNA recombination that is mediated by a mixture of lambda and E. coli-encoded recombination proteins. Recombination occurs between att sites on the interacting DNA molecules. For a description of att sites see Weisberg and Landy, Site-Specific Recombination in Phage Lambda, in Lambda II, p. 211-250, Cold Spring Harbor, NY: Cold Spring Harbor Press (1983). The DNA segments flanking the recombination sites are switched, such that after recombination, the att sites are hybrid sequences comprised of sequences donated by each parental vector. The recombination can occur between DNAs of any topology.

Att sites may be introduced into a sequence of interest by ligating the sequence of interest into an appropriate vector; generating a PCR product containing att B sites through the use of specific primers; generating a cDNA library cloned into an appropriate vector containing att sites; and the like.

Folding, as used herein, refers to the three-dimensional structure of polypeptides and proteins, where interactions between amino acid residues act to stabilize the structure. While non-covalent interactions are important in determining structure, usually the proteins of interest will have intra- and/or intermolecular covalent disulfide bonds formed by two cysteine residues. For naturally occurring proteins and polypeptides or derivatives and variants thereof, the proper folding is typically the arrangement that results in optimal biological activity, and can conveniently be monitored by assays for activity, e.g. ligand binding, enzymatic activity, etc.

In some instances, for example where the desired product is of synthetic origin, assays based on biological activity will be less meaningful. The proper folding of such molecules may be determined on the basis of physical properties, energetic considerations, modeling studies, and the like.

The expression host may be further modified by the introduction of sequences encoding one or more enzymes that enhance folding and disulfide bond formation, i.e. foldases, chaperonins, etc. Such sequences may be constitutively or inducibly expressed in the host cell, using vectors, markers, etc. as known in the art. Preferably the sequences, including transcriptional regulatory elements sufficient for the desired pattern of expression, are stably integrated in the yeast genome through a targeted methodology.

For example, the eukaryotic protein disulfide isomerase (“PDI”) is not only an efficient catalyst of protein cysteine oxidation and disulfide bond isomerization, but also exhibits chaperone activity. Co-expression of PDI can facilitate the production of active proteins having multiple disulfide bonds. Also of interest is the expression of immunoglobulin heavy chain binding protein (“BIP”); cyclophilin; and the like.

Cultured mammalian cells are exemplary hosts for production of the disclosed anti-CoV-S antibodies and antigen-binding fragments thereof. As mentioned, CHO cells are particularly suitable for expression of antibodies. Many procedures are known in the art for manufacturing monoclonal antibodies in mammalian cells. (See, Galfre, G. and Milstein, C., Methods Enzym., 73:3-46, 1981; Basalp et al., Turk. J. Biol., 24:189-196, 2000; Wurm, F. M., Nat. Biotechnol., 22:1393-1398, 2004; and Li et al., mAbs, 2(5):466-477, 2010). As mentioned in further detail infra, common host cell lines employed in mammalian monoclonal antibody manufacturing schemes include, but are not limited to, human embryonic retinoblast cell line PER.C6® (Crucell N. V., Leiden, The Netherlands), NS0 murine myeloma cells (Medical Research Council, London, UK), CV1 monkey kidney cell line, 293 human embryonic kidney cell line, BHK baby hamster kidney cell line, VERO African green monkey kidney cell line, human cervical carcinoma cell line HELA, MDCK canine kidney cells, BRL buffalo rat liver cells, W138 human lung cells, HepG2 human liver cells, MMT mouse mammary tumor cells, TRI cells, MRC5 cells, Fs4 cells, myeloma or lymphoma cells, or Chinese Hamster (Cricetulus griseus) Ovary (CHO) cells, and the like. Many different subclones or sub-cell lines of CHO cells known in the art that are useful and optimized for production of recombinant monoclonal antibodies, such as the DP12 (CHO K1 dhfr-) cell line, NS0 cells are a non-Ig secreting, non-light chain-synthesizing subclone of NS-1 cells that are resistant to azaguanine. Other Chinese Hamster and CHO cells are commercially available (from ATCC, etc.), including CHO-DXB11 (CHO-DUKX), CHO-pro3, CHO-DG44, CHO 1-15, CHO DP-12, Lec2, M1WT3, Lec8, pgsA-745, and the like, all of which are genetically altered to optimize the cell line for various parameters. Monoclonal antibodies are commonly manufactured using a batch fed method whereby the monoclonal antibody chains are expressed in a mammalian cell line and secreted into the tissue culture medium in a bioreactor. Medium (or feed) is continuously supplied to the bioreactor to maximize recombinant protein expression. Recombinant monoclonal antibody is then purified from the collected media. In some circumstances, additional steps are needed to reassemble the antibodies through reduction of disulfide bonds, etc. Such production methods can be scaled to be as large as 10,000 L in a single batch or more. It is now routine to obtain as much as 20 pg/cell/day through the use of such cell lines and methodologies, providing titers as high as 10 g/L or more, amounting to 15 to 100 kg from bioreactors of 10 kL to 25 kL. (Li et al., 2010). Various details of this production methodology, including cloning of the polynucleotides encoding the antibodies into expression vectors, transfecting cells with these expression vectors, selecting for transfected cells, and expressing and purifying the recombinant monoclonal antibodies from these cells are provided below.

For recombinant production of an anti-CoV-S antibody or antigen-binding fragment in mammalian cells, nucleic acids encoding the antibody or fragment thereof are generally inserted into a replicable vector for further cloning (amplification of the DNA) or for expression. DNA encoding the antibody is readily isolated or synthesized using conventional procedures (e.g., by using oligonucleotide probes that are capable of binding specifically to DNAs encoding the heavy and light chains of the antibody). The vector components generally include, but are not limited to, one or more of the following: a signal sequence, an origin of replication, one or more marker genes, an enhancer element, a promoter, and a transcription termination sequence. Selection of promoters, terminators, selectable markers, vectors, and other elements is a matter of routine design within the level of ordinary skill in the art. Many such elements are known in the art and are available through commercial suppliers.

The antibodies of this disclosure may be produced recombinantly not only directly, but also as a fusion polypeptide with a heterologous polypeptide, which is preferably a signal sequence or other polypeptide having a specific cleavage site at the N-terminus of the mature protein or polypeptide. The homologous or heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

Such expression vectors and cloning vectors will generally contain a nucleic acid sequence that enables the vector to replicate in one or more selected host cells. Typically, in cloning vectors this sequence is one that enables the vector to replicate independently of the host chromosomal DNA, and includes origins of replication or autonomously replicating sequences. Such sequences are well known for a variety of bacteria, yeast, and viruses, e.g., the origin of replication from the plasmid pBR322 is suitable for most Gram-negative bacteria, the 2mu plasmid origin is suitable for yeast, and various viral origins (Simian Virus 40 (“SV40”), polyoma, adenovirus, vesicular stomatitis virus (“VSV”), or bovine papillomavirus (“BPV”) are useful for cloning vectors in mammalian cells. Generally, the origin of replication component is not needed for mammalian expression vectors (the SV40 origin may typically be used only because it contains the early promoter).

These vectors will also typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” Examples of such dominant selection use the drugs neomycin, mycophenolic acid, and hygromycin. An exemplary selectable marker is a gene encoding resistance to the antibiotic neomycin. Selection is carried out in the presence of a neomycin-type drug, such as G-418 or the like. Those cells that are successfully transformed with a heterologous gene produce a protein conferring drug resistance and thus survive the selection regimen.

Selection systems can also be used to increase the expression level of the gene of interest, a process referred to as “amplification.” Amplification of transfectants typically occurs by culturing the cells in the presence of a low level of the selective agent and then increasing the amount of selective agent to select for cells that produce high levels of the products of the introduced genes. Exemplary suitable selectable markers for mammalian cells are those that enable the identification of cells competent to take up the antibody nucleic acid, such as dihydrofolate reductase (“DHFR”), thymidine kinase, metallothionein-I and -II, preferably primate metallothionein genes, adenosine deaminase, ornithine decarboxylase, etc.

For example, an amplifiable selectable marker for mammalian cells is dihydrofolate reductase, which confers resistance to methotrexate. Other drug resistance genes (e.g. hygromycin resistance, multi-drug resistance, puromycin acetyltransferase) can also be used. Cells transformed with the DHFR selection gene are first identified by culturing all of the transformants in a culture medium that contains methotrexate (“MTX”), a competitive antagonist of DHFR. An appropriate host cell when wild-type DHFR is employed is the Chinese hamster ovary (“CHO”) cell line deficient in DHFR activity.

Alternatively, host cells (particularly wild-type hosts that contain endogenous DHFR) transformed or co-transformed with DNA sequences encoding antibody, wild-type DHFR protein, and another selectable marker such as aminoglycoside 3′-phosphotransferase (“APH”) can be selected by cell growth in medium containing a selection agent for the selectable marker such as an aminoglycosidic antibiotic, e.g., kanamycin, neomycin, or G-418. See U.S. Pat. No. 4,965,199.

These vectors may comprise an enhancer sequence that facilitates transcription of a DNA encoding the antibody. Many enhancer sequences are known from mammalian genes (for example, globin, elastase, albumin, alpha-fetoprotein, and insulin). A frequently used enhancer is one derived from a eukaryotic cell virus. Examples thereof include the SV40 enhancer on the late side of the replication origin (bp 100-270), the cytomegalovirus early promoter enhancer, the polyoma enhancer on the late side of the replication origin, and adenovirus enhancers (See also Yaniv, Nature, 297:17-18 (1982) on enhancing elements for activation of eukaryotic promoters). The enhancer may be spliced into the vector at a position 5′ or 3′ to the antibody-encoding sequence, but is preferably located at a site 5′ from the promoter.

Expression and cloning vectors will also generally comprise a promoter that is recognized by the host organism and is operably linked to the antibody nucleic acid. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody transcription from vectors in mammalian host cells may be controlled, for example, by promoters obtained from the genomes of viruses such as polyoma virus, fowlpox virus, adenovirus (such as Adenovirus 2), BPV, avian sarcoma virus, cytomegalovirus, a retrovirus, hepatitis-B virus, and most preferably SV40, from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the BPV as a vector is disclosed in U.S. Pat. No. 4,419,446. A modification of this system is described in U.S. Pat. No. 4,601,978. See also Reyes et al., Nature, 297:598-601 (1982) on expression of human beta-interferon cDNA in mouse cells under the control of a thymidine kinase promoter from herpes simplex virus. Alternatively, the Rous sarcoma virus long terminal repeat can be used as the promoter.

Strong transcription promoters can be used, such as promoters from SV40, cytomegalovirus, or myeloproliferative sarcoma virus. See, e.g., U.S. Pat. No. 4,956,288 and U.S. Patent Publication No. 20030103986. Other suitable promoters include those from metallothionein genes (U.S. Pat. Nos. 4,579,821 and 4,601,978) and the adenovirus major late promoter. Expression vectors for use in mammalian cells include pZP-1, pZP-9, and pZMP21, which have been deposited with the American Type Culture Collection, 10801 University Blvd., Manassas, VA. USA under accession numbers 98669, 98668, and PTA-5266, respectively, and derivatives of these vectors.

Expression vectors used in eukaryotic host cells (yeast, fungus, insect, plant, animal, human, or a nucleated cell from other multicellular organism) will also generally contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the antibody. One useful transcription termination component is the bovine growth hormone polyadenylation region. See WO 94/11026 and the expression vector disclosed therein.

Suitable host cells for cloning or expressing the subject antibodies include prokaryote, yeast, or higher eukaryote cells described above. However, interest has been greatest in vertebrate cells, and propagation of vertebrate cells in culture has become a routine procedure. Examples of useful mammalian host cell lines are monkey kidney CV1 line transformed by SV40 (COS-1 (ATCC No. CRL 1650); and COS-7, ATCC CRL 1651); human embryonic kidney line (293 or 293 cells subcloned for growth in suspension culture, (ATCC No. CRL 1573; Graham et al., J. Gen. Virol., 36:59-72 (1977)); baby hamster kidney cells (BHK, ATCC CCL 10, ATCC No. CRL 1632; BHK 570, ATCC No. CRL 10314); CHO cells (CHO-K1, ATCC No. CCL 61; CHO-DG44, Urlaub et al., Proc. Natl. Acad. Sci. USA, 77:4216-4220 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N. Y. Acad. Sci., 383:44-68 (1982)); MRC 5 cells; FS4 cells; and a human hepatoma line (Hep G2). Additional suitable cell lines are known in the art and available from public depositories such as the American Type Culture Collection, Manassas, VA.

Host cells are transformed with the above-described expression or cloning vectors for antibody production and cultured in conventional nutrient media modified as appropriate for inducing promoters, selecting transformants, or amplifying the genes encoding the desired sequences as discussed supra.

The mammalian host cells used to produce the antibody of this disclosure may be cultured in a variety of media. Commercially available media such as Ham's F10 (Sigma-Aldrich Corporation, St. Louis, MO), Minimal Essential Medium ((“MEM” (Sigma-Aldrich Corporation, St. Louis, MO), Roswell Park Memorial Institute-1640 medium (“RPMI-1640”, Sigma-Aldrich Corporation, St. Louis, MO), and Dulbecco's Modified Eagle's Medium ((“DMEM” Sigma-Aldrich Corporation, St. Louis, MO) are suitable for culturing the host cells. In addition, any of the media described in Ham et al., Meth. Enz., 58:44 (1979), Barnes et al., Anal. Biochem., 102:255 (1980), U.S. Pat. Nos. 4,767,704; 4,657,866; 4,927,762; 4,560,655; or 5,122,469; WO 90/03430; WO 87/00195; or U.S. Pat. Reexam No. 30,985 can be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan. Methods of development and optimization of media and culture conditions are known in the art (See, Gronemeyer et al., Bioengineering, 1(4):188-212, 2014).

After culture conditions are optimized and a preferred cell line clone is selected, these cells are cultured (either adherent cells or suspension cultures) most typically in a batch-fed process in a bioreactor (many models are commercially available) that involves continuously feeding the cell culture with medium and feed, optimized for the particular cell line chosen and selected for this purpose. (See, Butler, M., Appl. Microbiol. Biotechnol., 68:283-291, 2005; and Kelley, B., mAb, 1(5):443-452, 2009). Perfusion systems are also available in which media and feed are continuously supplied to the culture while the same volume of media is being withdrawn from the bioreactor. (Wurm, 2004). Synthetic media, also commercially available, are available for growing cells in a batch-fed culture, avoiding the possibility of contamination from outside sources, such as with the use of animal components, such as bovine serum albumin, etc. However, animal-component-free hydrolysates are commercially available to help boost cell density, culture viability and productivity. (Li et al., 2010). Many studies have been performed in an effort to optimize cell culture media, including careful attention to head space available in roller bottles, redox potentials during growth and expression phases, presence of reducing agents to maintain disulfide bonds during production, etc. (See, for instance, Hutterer et al., mAbs, 5(4):608-613, 2013; and Mullan et al., BMC Proceed., 5(Suppl 8):P 110, 2011). Various methodologies have been developed to address the possibility of harmful oxidation during recombinant monoclonal antibody production. (See, for example, U.S. Pat. No. 8,574,869). Cultured cells may be grown by feeding nutrients continuously or as separately administered amounts. Often various process parameters such as cell concentration, pH, temperature, CO₂, dO₂, osmolality, amount of metabolites such as glucose, lactate, glutamine and glutamate, and the like, are monitored by the use of probes during the cell growth either on-line by direct connection to calibrated analyzers or off-line by intervention of operators. The culturing step also typically involves ensuring that the cells growing in culture maintain the transfected recombinant genes by any means known in the art for cell selection.

Following fermentation, i.e., upon reaching maximum cell growth and recombinant protein expression, the culturing step is typically followed by a harvesting step, whereby the cells are separated from the medium and a harvested cell culture media is thereby obtained. (See, Liu et al., mAbs, 2(5):480-499, 2010). Typically, various purification steps, involving column chromatography and the like, follow culturing to separate the recombinant monoclonal antibody from cell components and cell culture media components. The exact purification steps needed for this phase of the production of recombinant monoclonal antibodies depends on the site of expression of the proteins, i.e., in the cytosol of the cells themselves, or the more commonly preferred route of protein excreted into the cell culture medium. Various cell components may be separated using techniques known in the art such as differential centrifugation techniques, gravity-based cell settling, and/or size exclusion chromatograph/filtration techniques that can include tangential flow micro-filtration or depth filtration. (See, Pollock et al., Biotechnol. Bioeng., 110:206-219, 2013, and Liu et al., 2010). Centrifugation of cell components may be achieved on a large scale by use of continuous disk stack centrifuges followed by clarification using depth and membrane filters. (See, Kelley, 2009). Most often, after clarification, the recombinant protein is further purified by Protein A chromatography due to the high affinity of Protein A for the Fc domain of antibodies, and typically occurs using a low pH/acidification elution step (typically the acidification step is combined with a precautionary virus inactivation step). Flocculation and/or precipitation steps using acidic or cationic polyelectrolytes may also be employed to separate animal cells in suspension cultures from soluble proteins. (Liu et al., 2010). Lastly, anion- and cation-exchange chromatography, hydrophobic interaction chromatograph (“HIC”), hydrophobic charge induction chromatograph (HCIC), hydroxyapatite chromatography using ceramic hydroxyapatite (Ca₅(PO₄)₃OH)₂, and combinations of these techniques are typically used to polish the solution of recombinant monoclonal antibody. Final formulation and concentration of the desired monoclonal antibody may be achieved by use of ultracentrifugation techniques. Purification yields are typically 70 to 80%. (Kelley, 2009).

The terms “desired protein” or “desired antibody” herein are used interchangeably and refer generally to a parent antibody specific to a target, i.e., CoV-S or a chimeric or humanized antibody or a binding portion thereof derived therefrom as described herein. The term “antibody” is intended to include any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammals, chicken, other avians, etc., are considered to be “antibodies.” Examples thereof include chimeric antibodies, human antibodies and other non-human mammalian antibodies, humanized antibodies, single chain antibodies (such as scFvs), camelbodies, nanobodies, IgNAR (single-chain antibodies which may be derived from sharks, for example), small-modular immunopharmaceuticals (“SMIPs”), and antibody fragments such as Fabs, Fab′, F(ab′)₂, and the like (See Streltsov et al., Protein Sci., 14(11):2901-9 (2005); Greenberg et al., Nature, 374(6518):168-73 (1995); Nuttall et al., Mol. Immunol., 38(4):313-26 (2001); Hamers-Casterman et al., Nature, 363(6428):446-8 (1993); Gill et al., Curr. Opin. Biotechnol., (6):653-8 (2006)).

For example, antibodies or antigen-binding fragments thereof may be produced by genetic engineering. In this technique, as with other methods, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones that co-express a heavy and light chain (resembling the Fab fragment or antigen-binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (“aa”) substitutions, additions, or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc). Variants also include fragments of the polypeptides disclosed herein, particularly biologically active fragments and/or fragments corresponding to functional domains. Techniques for in vitro mutagenesis of cloned genes are known. Also included in the subject disclosure are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the VL and VH regions, obtained from antibody producing cells of one species with the constant light and heavy chain regions from another. Typically, chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the disclosure may be selected from IgG1, IgG2, IgG3, and IgG4 constant regions.

Humanized antibodies are engineered to contain even more human-like immunoglobulin domains, and incorporate only the complementarity determining regions of the animal-derived antibody. This is accomplished by carefully examining the sequence of the hyper-variable loops of the variable regions of the monoclonal antibody and fitting them to the structure of the human antibody chains. Although facially complex, the process is straightforward in practice. See, e.g., U.S. Pat. No. 6,187,287, incorporated fully herein by reference.

In addition to entire immunoglobulins (or their recombinant counterparts), immunoglobulin fragments comprising the epitope binding site (e.g., Fab′, F(ab′)2, or other fragments) may be synthesized. “Fragment” or minimal immunoglobulins may be designed utilizing recombinant immunoglobulin techniques. For instance, “Fv” immunoglobulins for use in the present disclosure may be produced by synthesizing a fused variable light chain region and a variable heavy chain region. Combinations of antibodies are also of interest, e.g. diabodies, which comprise two distinct Fv specificities. In another embodiment, small molecule immunopharmaceuticals (“SMIPs”), camelbodies, nanobodies, and IgNAR are encompassed by immunoglobulin fragments.

Immunoglobulins and fragments thereof may be modified post-translationally, e.g. to add effector moieties such as chemical linkers, detectable moieties, such as fluorescent dyes, enzymes, toxins, substrates, bioluminescent materials, radioactive materials, chemiluminescent moieties, and the like, or specific binding moieties, such as streptavidin, avidin, or biotin, and the like may be utilized in the methods and compositions of the present disclosure. Examples of additional effector molecules are provided infra.

A polynucleotide sequence “corresponds” to a polypeptide sequence if translation of the polynucleotide sequence in accordance with the genetic code yields the polypeptide sequence (i.e., the polynucleotide sequence “encodes” the polypeptide sequence), one polynucleotide sequence “corresponds” to another polynucleotide sequence if the two sequences encode the same polypeptide sequence.

A “heterologous” region or domain of a DNA construct is an identifiable segment of DNA within a larger DNA molecule that is not found in association with the larger molecule in nature. Thus, when the heterologous region encodes a mammalian gene, the DNA flanking the gene usually does not flank the mammalian genomic DNA in the genome of the source organism. Another example of a heterologous region is a construct where the coding sequence itself is not found in nature (e.g., a cDNA where the genomic coding sequence contains introns or synthetic sequences having codons different than the native gene). Allelic variations or naturally-occurring mutational events do not give rise to a heterologous region of DNA as defined herein.

A “coding sequence” is an in-frame sequence of codons that correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide. A polyadenylation signal and transcription termination sequence will usually be located 3′ to the coding sequence. A “promoter sequence” is a DNA regulatory region capable of initiating transcription of a downstream (3′ direction) coding sequence, and typically contain additional sites for binding of regulatory molecules, e.g., transcription factors, that affect the transcription of the coding sequence. A coding sequence is “under the control” of the promoter sequence or “operatively linked” to the promoter when RNA polymerase binds the promoter sequence in a cell and transcribes the coding sequence into mRNA, which is then in turn translated into the protein encoded by the coding sequence.

The general structure of antibodies in vertebrates now is well understood. See Edelman, G. M., Ann. N.Y. Acad. Sci., 190:5 (1971). Antibodies consist of two identical light polypeptide chains of molecular weight approximately 23,000 daltons (the “light chain”), and two identical heavy chains of molecular weight 53,000-70,000 (the “heavy chain”). The four chains are joined by disulfide bonds in a “Y” configuration wherein the light chains bracket the heavy chains starting at the mouth of the “Y” configuration. The “branch” portion of the “Y” configuration is designated the Fab region; the stem portion of the “Y” configuration is designated the Fc region. The amino acid sequence orientation runs from the N-terminal end at the top of the “Y” configuration to the C-terminal end at the bottom of each chain. The N-terminal end possesses the variable region having specificity for the antigen that elicited it, and is approximately 100 amino acids in length, there being slight variations between light and heavy chain and from antibody to antibody.

The variable region is linked in each chain to a constant region that extends the remaining length of the chain and that within a particular class of antibody does not vary with the specificity of the antibody (i.e., the antigen eliciting it). There are five known major classes of constant regions that determine the class of the immunoglobulin molecule (IgG, IgM, IgA, IgD, and IgE corresponding to γ, μ, α, δ, and ε (gamma, mu, alpha, delta, or epsilon) heavy chain constant regions). The constant region or class determines subsequent effector function of the antibody, including activation of complement (see Kabat, E. A., Structural Concepts in Immunology and Immunochemistry, 2nd Ed., p. 413-436, New York, NY: Holt, Rinehart, Winston (1976)), and other cellular responses (see Andrews et al., Clinical Immunology, pp. 1-18, W. B. Sanders, Philadelphia, PA (1980); Kohl et al., Immunology, 48:187 (1983)); while the variable region determines the antigen with which it will react. Light chains are classified as either κ (kappa) or λ (lambda). Each heavy chain class can be prepared with either kappa or lambda light chain. The light and heavy chains are covalently bonded to each other, and the “tail” portions of the two heavy chains are bonded to each other by covalent disulfide linkages when the immunoglobulins are generated either by hybridomas or by B-cells.

The expression “variable region” or “VR” refers to the domains within each pair of light and heavy chains in an antibody that are involved directly in binding the antibody to the antigen. Each heavy chain has at one end a variable region (VH) followed by a number of constant domains. Each light chain has a variable region (VL) at one end and a constant domain at its other end; the constant domain of the light chain is aligned with the first constant domain of the heavy chain, and the light chain variable domain is aligned with the variable domain of the heavy chain.

The expressions “complementarity-determining region,” “hypervariable region,” or “CDR” refer to one or more of the hyper-variable or complementarity-determining regions (“CDRs”) found in the variable regions of light or heavy chains of an antibody (See Kabat et al., Sequences of Proteins of Immunological Interest, 4^{th} ed., Bethesda, MD: U.S. Dept. of Health and Human Services, Public Health Service, National Institutes of Health (1987)). These expressions include the hypervariable regions as defined by Kabat et al., (Sequences of Proteins of Immunological Interest, NIH Publication No. 91-3242, Bethesda, MD: U.S. Dept. of Health and Human Services, National Institutes of Health (1983)) or the hypervariable loops in 3-dimensional structures of antibodies (Chothia and Lesk, J. Mol. Biol., 196:901-917 (1987)). The CDRs in each chain are held in close proximity by framework regions (“FRs”) and, with the CDRs from the other chain, contribute to the formation of the antigen binding site. Within the CDRs there are select amino acids that have been described as the selectivity determining regions (“SDRs”) that represent the critical contact residues used by the CDR in the antibody-antigen interaction (see Kashmiri et al., Methods, 36(1):25-34 (2005)).

An “isolated antibody”, as used herein, is intended to refer to an antibody that is substantially free of other antibodies having different antigenic specificities (e.g., an isolated antibody that specifically binds CoV-S is substantially free of antibodies that specifically bind antigens other than CoV-S). An isolated antibody that specifically binds CoV-S may, however, have cross-reactivity to other antigens, such as CoV-S molecules from other species. Moreover, an isolated antibody may be substantially free of other cellular material and/or chemicals.

The phrase “specifically binds to CoV-S” as used herein, refers to the ability of an anti-CoV-S antibody or antigen-binding fragment thereof to interact with CoV-S with a dissociated constant (KD) of, for example, about 1,000 nM or less, about 500 nM or less, about 200 nM or less, about 100 nM or less, about 75 nM or less, about 25 nM or less, about 10 nM or less, about 1 nM or less, about 100 pM nM or less, about 10 pM nM or less, about 1 pM or less, or about 0.1 pM or less. In another embodiment, the phrase “specifically binds to CoV-S”, as used herein, refers to the ability of an anti-CoV-S antibody or antigen-binding fragment thereof to interact with CoV-S with a dissociation constant (KD) of between about 0.1 pM to 1,000 nM, between about 1 pM to 500 nM, between about 10 pM to 100 nM, between about 0.1 nM to 50 nM, or between about 1 nM to 50 nM. In one embodiment, KD is determined by surface plasmon resonance, ELISAs, radioimmunoassays, bio-layer interferometry (BLI), or by any other methods known in the art.

An “epitope” or “binding site” is an area or region on an antigen to which an antigen-binding peptide (such as an antibody) specifically binds. A protein epitope may comprise amino acid residues directly involved in the binding (also called immunodominant component of the epitope) and other amino acid residues, which are not directly involved in the binding, such as amino acid residues that are effectively blocked by the specifically antigen binding peptide (in other words, the amino acid residue is within the “footprint” of the specifically antigen binding peptide). The term epitope herein includes both types of amino acid binding sites in any particular region of CoV-S, e.g., SARS-CoV-S or SARS-CoV-2-S, that specifically binds to an anti-CoV-S antibody. In some embodiments, the epitope is a conserved site within the spike protein, e.g., SARS-CoV-S or SARS-CoV-2-S, e.g., the CR3022 site and the N343 proteoglycan site in the receptor binding domain (RBD), or the S2 domain. CoV-S may comprise a number of different epitopes, which may include, without limitation, (1) linear peptide antigenic determinants, (2) conformational antigenic determinants that consist of one or more non-contiguous amino acids located near each other in a mature CoV-S conformation; and (3) post-translational antigenic determinants that consist, either in whole or part, of molecular structures covalently attached to a CoV-S protein such as carbohydrate groups. In particular, the term “epitope” includes the specific residues in a protein or peptide, e.g., CoV-S, which are involved in the binding of an antibody to such protein or peptide as determined by known and accepted methods such as alanine scanning techniques or the use of various S protein portions with varying lengths.

The phrase that an antibody (e.g., first antibody) binds “substantially” or “at least partially” the same epitope as another antibody (e.g., second antibody) means that the epitope binding site for the first antibody comprises at least 10%, 20%, 30%, 40%, 50%, 60%, 70%, 80%, 90%, or more of the amino acid residues on the antigen that constitutes the epitope binding site of the second antibody. Also, that a first antibody binds substantially or partially the same or overlapping epitope as a second antibody means that the first and second antibodies compete in binding to the antigen, as described above. Thus, the term “binds to substantially the same epitope or determinant as” a monoclonal antibody means that an antibody “competes” with the antibody.

The phrase “binds to the same or overlapping epitope or determinant as” an antibody of interest means that an antibody “competes” with said antibody of interest for at least one, (e.g., at least 2, at least 3, at least 4, at least 5) or all residues on CoV-S to which said antibody of interest specifically binds. The identification of one or more antibodies that bind(s) to substantially or essentially the same epitope as the monoclonal antibodies described herein can be readily determined using alanine scanning. Additionally, any one of variety of immunological screening assays in which antibody competition can be assessed. A number of such assays are routinely practiced and well known in the art (see, e.g., U.S. Pat. No. 5,660,827, issued Aug. 26, 1997, which is specifically incorporated herein by reference). It will be understood that actually determining the epitope to which an antibody described herein binds is not in any way required to identify an antibody that binds to the same or substantially the same or overlapping epitope as the monoclonal antibody described herein.

For example, where the test antibodies to be examined are obtained from different source animals, or are even of a different Ig isotype, a simple competition assay may be employed in which the control antibody is mixed with the test antibody and then applied to a sample containing CoV-S. Protocols based upon ELISAs, radioimmunoassays, Western blotting, and the use of BIACORE® (GE Healthcare Life Sciences, Marlborough, MA) analysis are suitable for use in such simple competition studies.

In certain embodiments, the control anti-CoV-S antibody is pre-mixed with varying amounts of the test antibody (e.g., in ratios of about 1:1, 1:2, 1:10, or about 1:100) for a period of time prior to applying to the CoV-S (e.g., SARS-CoV-S or SARS-CoV-2-S) antigen sample. In other embodiments, the control and varying amounts of test antibody can simply be added separately and admixed during exposure to the SARS-CoV-S or SARS-CoV-2-S antigen sample. As long as bound antibodies can be distinguished from free antibodies (e.g., by using separation or washing techniques to eliminate unbound antibodies) and control antibody from the test antibody (e.g., by using species specific or isotype specific secondary antibodies or by specifically labeling the control antibody with a detectable label) it can be determined if the test antibody reduces the binding of the control antibody to the SARS-CoV-S or SARS-CoV-2-S antigens, indicating that the test antibody recognizes substantially the same epitope as the control anti-CoV-S antibody. The binding of the (labeled) control antibody in the presence of a completely irrelevant antibody (that does not bind CoV-S) can serve as the control high value. The control low value can be obtained by incubating the labeled control antibody with the same but unlabeled control antibody, where competition would occur and reduce binding of the labeled antibody. In a test assay, a significant reduction in labeled antibody reactivity in the presence of a test antibody is indicative of a test antibody that recognizes substantially the same epitope, i.e., one that competes with the labeled control antibody. For example, any test antibody that reduces the binding of the control antibody to SARS-CoV-S or SARS-CoV-2-S by at least about 50%, such as at least about 60%, or more preferably at least about 70% (e.g., about 65-100%), at any ratio of test antibody between about 1:1 or 1:10 and about 1:100 is considered to be an antibody that binds to substantially the same or overlapping epitope or determinant as the control antibody.

Preferably, such test antibody will reduce the binding of the control antibody to SARS-CoV-S or SARS-CoV-2-S (or another CoV-S) antigen preferably at least about 50%, at least about 60%, at least about 80%, or at least about 90% (e.g., about 95%) of the binding of the control antibody observed in the absence of the test antibody.

A simple competition assay in which a test antibody is applied at saturating concentration to a surface onto which SARS-CoV-S or SARS-CoV-2-S (or another CoV-S) is immobilized also may be advantageously employed. The surface in the simple competition assay is preferably a BIACORE® (GE Healthcare Life Sciences, Marlborough, MA) chip (or other media suitable for surface plasmon resonance (“SPR”) analysis). The binding of a control antibody that binds SARS-CoV-S or SARS-CoV-2-S to the COV-S-coated surface is measured. This binding to the SARS-CoV-S- or SARS-CoV-2-S-containing surface of the control antibody alone is compared with the binding of the control antibody in the presence of a test antibody. A significant reduction in binding to the SARS-CoV-S- or SARS-CoV-2-S-containing surface by the control antibody in the presence of a test antibody indicates that the test antibody recognizes substantially the same epitope as the control antibody such that the test antibody “competes” with the control antibody. Any test antibody that reduces the binding of control antibody by at least about 20% or more, at least about 40%, at least about 50%, at least about 70%, or more, can be considered to be an antibody that binds to substantially the same epitope or determinant as the control antibody. Preferably, such test antibody will reduce the binding of the control antibody to SARS-CoV-S or SARS-CoV-2-S by at least about 50% (e.g., at least about 60%, at least about 70%, or more). It will be appreciated that the order of control and test antibodies can be reversed; i.e. the control antibody can be first bound to the surface and then the test antibody is brought into contact with the surface thereafter in a competition assay. Preferably, the “sandwich-style” binding assay infra is used. Alternatively, the antibody having greater affinity for SARS-CoV-S or SARS-CoV-2-S antigen is bound to the SARS-CoV-S- or SARS-CoV-2-S-containing surface first, as it will be expected that the decrease in binding seen for the second antibody (assuming the antibodies are competing) will be of greater magnitude. Further examples of such assays are provided in e.g., Saunal and Regenmortel, J. Immunol. Methods, 183:33-41 (1995), the disclosure of which is incorporated herein by reference.

In addition, whether an antibody binds the same or overlapping epitope(s) on COV-S as another antibody or the epitope bound by a test antibody may in particular be determined using a Western-blot based assay. In this assay a library of peptides corresponding to the antigen bound by the antibody, the CoV-S protein, is made, that comprise overlapping portions of the protein, typically 10-25, 10-20, or 10-15 amino acids long. These different overlapping amino acid peptides encompassing the CoV-S sequence are synthesized and covalently bound to a PEPSPOTS™ nitrocellulose membrane (JPT Peptide Technologies, Berlin, Germany). Blots are then prepared and probed according to the manufacturer's recommendations.

Essentially, the immunoblot assay then detects by fluorometric means what peptides in the library bind to the test antibody and thereby can identify what residues on the antigen, i.e., COV-S, interact with the test antibody. (See U.S. Pat. No. 7,935,340, incorporated by reference herein).

Various epitope mapping techniques are known in the art. By way of example, X-ray co-crystallography of the antigen and antibody; NMR; SPR (e.g., at 250 or 37° C.); array-based oligo-peptide scanning (or “pepscan analysis”); site-directed mutagenesis (e.g., alanine scanning); mutagenesis mapping; hydrogen-deuterium exchange; phage display; and limited proteolysis are all epitope mapping techniques that are well known in the art (See, e.g., Epitope Mapping Protocols: Second Edition, Methods in Molecular Biology, editors Mike Schutkowski and Ulrich Reineke, 2^{nd} Ed., New York, NY: Humana Press (2009), and Epitope Mapping Protocols, Methods in Molecular Biology, editor Glenn Morris, 1^{st} Ed., New York, NY: Humana Press (1996), both of which are herein incorporated by referenced in their entirety).

The identification of one or more antibodies that bind(s) to substantially or essentially the same epitope as the monoclonal antibodies described herein, e.g., VYD2311, can be readily determined using any one of variety of immunological screening assays in which antibody competition can be assessed. A number of such assays are routinely practiced and well known in the art (see, e.g., U.S. Pat. No. 5,660,827, issued Aug. 26, 1997, which is incorporated herein by reference). It will be understood that determining the epitope to which an antibody described herein binds is not in any way required to identify an antibody that binds to the same or substantially the same epitope as the monoclonal antibody described herein.

For example, where the test antibodies to be examined are obtained from different source animals, or are even of a different Ig isotype, a simple competition assay may be employed in which the control antibody (for example, VYD2311) is mixed with the test antibody and then applied to a sample containing either or both SARS-CoV-S or SARS-CoV-2-S, each of which is known to be bound by the antibodies. Protocols based upon ELISAs, radioimmunoassays, Western blotting, and BIACORE® (GE Healthcare Life Sciences, Marlborough, MA) analysis (as described in the Examples section herein) are suitable for use in such simple competition studies.

In certain embodiments, the method comprises pre-mixing the control antibody with varying amounts of the test antibody (e.g., in ratios of about 1:1, 1:2, 1:10, or about 1:100) for a period of time prior to applying to the CoV-S antigen sample. In other embodiments, the control and varying amounts of test antibody can be added separately and admixed during exposure to the CoV-S antigen sample. As long as bound antibodies can be distinguished from free antibodies (e.g., by using separation or washing techniques to eliminate unbound antibodies) and control antibody from the test antibody (e.g., by using species specific or isotype specific secondary antibodies or by specifically labelling the control antibody with a detectable label), the method can be used to determine that the test antibody reduces the binding of the control antibody to the CoV-S antigen, indicating that the test antibody recognizes substantially the same epitope as the control antibody (e.g., VYD2311). The binding of the (labeled) control antibody in the presence of a completely irrelevant antibody (that does not bind CoV-S) can serve as the control high value. The control low value can be obtained by incubating the labeled control antibody with the same but unlabeled control antibody, where competition would occur and reduce binding of the labeled antibody. In a test assay, a significant reduction in labeled antibody reactivity in the presence of a test antibody is indicative of a test antibody that recognizes substantially the same epitope, i.e., one that competes with the labeled control antibody. For example, any test antibody that reduces the binding of VYD2311 to both of SARS-CoV-S or SARS-CoV-2-S antigens by at least about 50%, such as at least about 60%, or more preferably at least about 70% (e.g., about 65-100%), at any ratio of control antibody: test antibody between about 1:1 or 1:10 and about 1:100 is considered to be an antibody that binds to substantially the same epitope or determinant as the antibody. Preferably, such test antibody will reduce the binding of VYD2311 to at least one, preferably each, of the SARS-CoV-S or SARS-CoV-2-S antigens preferably at least about 50%, at least about 60%, at least about 80% or at least about 90% (e.g., about 95%) of the binding of VYD2311 observed in the absence of the test antibody. These methods can be adapted to identify and/or evaluate antibodies that compete with other control antibodies.

A simple competition assay in which a test antibody is applied at saturating concentration to a surface onto which either SARS-CoV-S or SARS-CoV-2-S, or both, are immobilized also may be advantageously employed. The surface in the simple competition assay is preferably of a media suitable for OCTET® and/or PROTEON®. The binding of a control antibody (e.g., VYD2311) to the CoV-S-coated surface is measured. This binding to the CoV-S-containing surface of the control antibody alone is compared with the binding of the control antibody in the presence of a test antibody. A significant reduction in binding to the CoV-S-containing surface by the control antibody in the presence of a test antibody indicates that the test antibody recognizes substantially the same epitope as the control antibody such that the test antibody “competes” with the control antibody. Any test antibody that reduces the binding of control antibody (e.g., VYD2311) to both of SARS-CoV-S and SARS-CoV-2-S antigens by at least about 20% or more, at least about 40%, at least about 50%, at least about 70%, or more, can be considered to be an antibody that binds to substantially the same epitope or determinant as the control antibody (e.g., VYD2311). Preferably, such test antibody will reduce the binding of the control antibody (e.g., VYD2311) to the CoV-S antigen by at least about 50% (e.g., at least about 60%, at least about 70%, or more). It will be appreciated that the order of control and test antibodies can be reversed; i.e. the control antibody can be first bound to the surface and then the test antibody is brought into contact with the surface thereafter in a competition assay. Preferably, the antibody having higher affinity for SARS-CoV-S and SARS-CoV-2-S is bound to the CoV-S-containing surface first, as it will be expected that the decrease in binding seen for the second antibody (assuming the antibodies are competing) will be of greater magnitude. Further examples of such assays are provided in, e.g., Saunal and Regenmortel, J. Immunol. Methods, 183:33-41 (1989), the disclosure of which is incorporated herein by reference.

Determination of whether an antibody, antigen-binding fragment thereof, or antibody derivative, e.g., an affinity-matured antibody or antigen binding fragment of any of the anti-CoV-S antibodies exemplified herein, binds within one of the epitope regions defined above can be carried out in ways known to the person skilled in the art. In another example of such mapping/characterization methods, an epitope region for an anti-CoV-S antibody may be determined by epitope “footprinting” using chemical modification of the exposed amines/carboxyls in the SARS-CoV-S and SARS-CoV-2-S protein. One specific example of such a foot-printing technique is the use of hydrogen-deuterium exchange detected by mass spectrometry (“HXMS”), wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry (See, e.g., Ehring H., Analytical Biochemistry, 267(2):252-259 (1999) and Engen, J. R. & Smith, D. L., Anal. Chem., 73:256A-265A (2001)). Another example of a suitable epitope identification technique is nuclear magnetic resonance epitope mapping (“NMR”), where typically the position of the signals in two-dimensional NMR spectras of the free antigen and the antigen complexed with the antigen binding peptide, such as an antibody, are compared. The antigen typically is selectively isotopically labeled with ¹⁵N so that only signals corresponding to the antigen and no signals from the antigen binding peptide are seen in the NMR-spectrum. Antigen signals originating from amino acids involved in the interaction with the antigen binding peptide typically will shift position in the spectras of the complex compared to the spectras of the free antigen, and the amino acids involved in the binding can be identified that way. See, e.g., Ernst Schering Res. Found. Workshop, (44):149-67 (2004); Huang et al., J. Mol. Biol., 281(1):61-67 (1998); and Saito and Patterson, Methods, 9(3):516-24 (1996). Epitope mapping/characterization also can be performed using mass spectrometry (“MS”) methods (See, e.g., Downard, J. Mass Spectrom., 35(4):493-503 (2000) and Kiselar and Downard, Anal. Chem., 71(9):1792-801 (1999)).

Protease digestion techniques also can be useful in the context of epitope mapping and identification. Antigenic determinant-relevant regions/sequences can be determined by protease digestion, e.g. by using trypsin in a ratio of about 1:50 to SARS-CoV-S or SARS-CoV-2-S overnight (“o/n”) digestion at 37° C. and pH 7-8, followed by mass spectrometry (“MS”) analysis for peptide identification. The peptides protected from trypsin cleavage by the anti-CoV-S antibody can subsequently be identified by comparison of samples subjected to trypsin digestion and samples incubated with antibody and then subjected to digestion by e.g. trypsin (thereby revealing a footprint for the antibody). Other enzymes like chymotrypsin or pepsin can be used in similar epitope characterization methods. Moreover, enzymatic digestion can provide a quick method for analyzing whether a potential antigenic determinant sequence is within a region of CoV-S in the context of a CoV-S-binding polypeptide. If the polypeptide is not surface exposed, it is most likely not relevant in terms of immunogenicity/antigenicity (See, e.g., Manca, Ann. 1st. Super. Sanita., 27(1):15-9 (1991) for a discussion of similar techniques).

Site-directed mutagenesis is another technique useful for characterization of a binding epitope. For example, in “alanine-scanning” site-directed mutagenesis (also known as alanine scanning, alanine scanning mutagenesis, alanine scanning mutations, combinatorial alanine scanning, or creation of alanine point mutations, for example), each residue within a protein segment is replaced with an alanine residue (or another residue such as valine where alanine is present in the wild-type sequence) through such methodologies as direct peptide or protein synthesis, site-directed mutagenesis, the GENEART™ Mutagenesis Service (Thermo Fisher Scientific, Waltham, MA U.S.A.) or shotgun mutagenesis, for example. A series of single point mutants of the molecule is thereby generated using this technique; the number of mutants generated is equivalent to the number of residues in the molecule, each residue being replaced, one at a time, by a single alanine residue. Alanine is generally used to replace native (wild-type) residues because of its non-bulky, chemically inert, methyl functional group that can mimic the secondary structure preferences that many other amino acids may possess. Subsequently, the effects replacing a native residue with an alanine has on binding affinity of an alanine scanning mutant and its binding partner can be measured using such methods as, but not limited to, SPR binding experiments. If a mutation leads to a significant reduction in binding affinity, it is most likely that the mutated residue is involved in binding. Monoclonal antibodies specific for structural epitopes (i.e., antibodies that do not bind the unfolded protein) can be used as a positive control for binding affinity experiments to verify that the alanine-replacement does not influence the overall tertiary structure of the protein (as changes to the overall fold of the protein may indirectly affect binding and thereby produce a false positive result). See, e.g., Clackson and Wells, Science, 267:383-386 (1995); Weiss et al., Proc. Natl. Acad. Sci. USA, 97(16):8950-8954 (2000); and Wells, Proc. Natl. Acad. Sci. USA, 93:1-6 (1996). Example 5 identifies the specific epitope or residues of CoV-S which specifically interact with the anti-CoV-S antibodies disclosed herein.

Electron microscopy can also be used for epitope “footprinting”. For example, Wang et al., Nature, 355:275-278 (1992) used coordinated application of cryoelectron microscopy, three-dimensional image reconstruction, and X-ray crystallography to determine the physical footprint of a Fab-fragment on the capsid surface of native cowpea mosaic virus.

Other forms of “label-free” assay for epitope evaluation include SPR (sold commercially as the BIACORE® system, GE Healthcare Life Sciences, Marlborough, MA) and reflectometric interference spectroscopy (“RifS”) (See, e.g., Fagerstam et al., Journal of Molecular Recognition, 3:208-14 (1990); Nice et al., J. Chromatogr., 646:159-168 (1993); Leipert et al., Angew. Chem. Int. Ed., 37:3308-3311 (1998); Kroger et al., Biosensors and Bioelectronics, 17:937-944 (2002)).

The expressions “framework region” or “FR” refer to one or more of the framework regions within the variable regions of the light and heavy chains of an antibody (See Kabat et al., Sequences of Proteins of Immunological Interest, 4^{th} edition, Bethesda, MD: U.S. Dept. of Health and Human Services, Public Health Service, National Institutes of Health (1987)). These expressions include those amino acid sequence regions interposed between the CDRs within the variable regions of the light and heavy chains of an antibody.

The term “Fe region” is used to define a C-terminal region of an immunoglobulin heavy chain. The “Fe region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th edition, Bethesda, MD: U.S. Dept. of Health and Human Services, Public Health Service, National Institutes of Health (1991). The Fc region of an immunoglobulin generally comprises two constant domains, CH2 and CH3.

The terms “Fe receptor” and “FcR” describe a receptor that binds to the Fc region of an antibody. The preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, Ann. Rev. Immunol., 9:457-92 (1991); Capel et al., Immunomethods, 4:25-34 (1994); and de Haas et al., J. Lab. Clin. Med., 126:330-41 (1995). “FcR” also includes the neonatal receptor, FcRn, which is responsible for the transfer of maternal IgGs to the fetus (Guyer et al., J. Immunol., 117:587 (1976); and Kim et al., J. Immunol., 24:249 (1994)), and which primarily functions to modulate and/or extend the half-life of antibodies in circulation. To the extent that the disclosed anti-CoV-S antibodies are aglycosylated, as a result of the expression system and/or sequence, the subject antibodies are expected to bind FcRn receptors, but not to bind (or to minimally bind) Fcγ receptors.

A “functional Fc region” possesses at least one effector function of a native sequence Fc region. Exemplary “effector functions” include C1q binding; complement dependent cytotoxicity (“CDC”); Fc receptor binding; antibody-dependent cell-mediated cytotoxicity (“ADCC”); phagocytosis; down-regulation of cell surface receptors (e.g. B cell receptor (“BCR”)), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence that differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95%, at least about 96%, at least about 97%, at least about 98%, or at least about 99% sequence identity therewith.

In some embodiments, the Fc region of an antibody or antigen-binding antibody fragment of the present disclosure may bind to an Fc receptor (FcR). The FcR may be, but is not limited to, Fe gamma receptor (FcgR), FcgRI, FcgRIIA, FcgRIIB1, FcgRIIB2, FcgRIIIA, FcgRIIIB, Fe epsilon receptor (FceR), FceRI, FceRII, Fe alpha receptor (FcaR), FcaRI, Fe alpha/mu receptor (Fca/mR), or neonatal Fc receptor (FcRn). The Fe may be an IgM, IgD, IgG, IgE, or IgA isotype. An IgG isotype may be an IgG1, IgG2, IgG3, or IgG4.

Certain amino acid modifications in the Fc region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half-life (Wang X. et al., Protein Cell. 2018 January; 9(1): 63-73; Dall'Acqua W. F. et al., J Biol Chem. 2006 Aug. 18; 281(33):23514-24. Epub 2006 Jun. 21; Monnet C. et al, Front Immunol. 2015 Feb. 4; 6:39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289(6): 3571-3590).

Any of the antibody variable region sequences disclosed herein may be used in combination with a wild-type (WT) Fe or a variant Fe.

An IgG1-type Fe optionally may comprise one or more amino acid substitutions. Such substitutions may include, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V3051, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y, M428L, N434S, L328F, M252Y, S254T, T256E, and/or any combination thereof (the residue numbering is according to the EU index as in Kabat) (Dall'Acqua W. F. et al., J Biol Chem. 2006 Aug. 18; 281(33):23514-24. Epub 2006 Jun. 21; Wang X. et al., Protein Cell. 2018 January; 9(1): 63-73), or for example, N434A, Q438R, S440E, L432D, N434L, and/or any combination thereof (the residue numbering according to EU numbering). The Fc region may further comprise one or more additional amino acid substitutions. Such substitutions may include but are not limited to A330L, L234F, L235E, P3318, and/or any combination thereof (the residue numbering is according to the EU index as in Kabat). Specific exemplary substitution combinations for an IgG1-type Fe include, but not limited to: M252Y, S254T, and T256E (“YTE” variant); M428L and N434A (“LA” variant), M428L and N434S (“LS” variant); M428L, N434A, Q438R, and S440E (“LA-RE” variant); L432D and N434L (“DEL” variant); and L234A, L235A, L432D, and N434L (“LALA-DEL” variant) (the residue numbering is according to the EU index as in Kabat).

When the Ab is an IgG2, the Fc region optionally may comprise one or more amino acid substitutions. Such substitutions may include but are not limited to P238S, V234A, G237A, H268A, H268Q, H268E, V309L, N297A, N297Q, A330S, P331S, C232S, C233S, M252Y, S254T, T256E, and/or any combination thereof (the residue numbering is according to the EU index as in Kabat). The Fc region optionally may further comprise one or more additional amino acid substitutions. Such substitutions may include but are not limited to M252Y, S254T, T256E, and/or any combination thereof (the residue numbering is according to the EU index as in Kabat).

An IgG3-type Fc region optionally may comprise one or more amino acid substitutions. Such substitutions may include but are not limited to E235Y (the residue numbering is according to the EU index as in Kabat).

An IgG4-type Fc region optionally may comprise one or more amino acid substitutions. Such substitutions may include but are not limited to, E233P, F234V, L235A, G237A, E318A, S228P, L236E, S241P, L248E, T394D, M252Y, S254T, T256E, N297A, N297Q, and/or any combination thereof (the residue numbering is according to the EU index as in Kabat). The substitution may be, for example, S228P (the residue numbering is according to the EU index as in Kabat).

In some cases, the glycan of the human-like Fc region may be engineered to modify the effector function (for example, see Li T. et al., Proc Natl Acad Sci USA. 2017 Mar. 28; 114(13):3485-3490. doi: 10.1073/pnas.1702173114. Epub 2017 Mar. 13).

### B. Anti-CoV-S Antibodies and Binding Fragments Thereof Having Binding Activity for CoV-S

The present disclosure provides antibodies, or antigen-binding fragments thereof, that display improved properties, e.g., broad activity against all SARS-CoV-2 variants of concern (VOCs) described to date (including the BA.1/Omicron variant), and improved thermal and colloidal stability in vitro.

In some embodiments, an antibody or antigen-binding fragment thereof is capable of binding to the spike protein of a coronavirus (CoV-S). In some embodiments, the CoV-S is the spike protein of SARS-CoV (“SARS-CoV-S”) and/or the spike protein of SARS-CoV-2 (“SARS-CoV-2-S”).

In certain embodiments, an antibody or antigen-binding fragment thereof is capable of binding to a SARS-CoV-2 variant. In some embodiments, the SARS-CoV-2-S is a B.1.1.7 variant, a B.1.351 variant, a B.1.1.28 variant, a B.1.429 variant, a P.1 variant, a B.1.617 variant, a B.1.617.2 variant, a C.37 variant, a 1.621 variant, a AY.1 variant, a 1.623 variant, a C.36 variant, a A.27 variant, a AV.1 variant, a B.1.1.482 variant, a B.1.1.523 variant, a B.1.427 variant, a AY.4 variant, a AY.11 variant, a D614G variant, a B.1.1.529/BA.1 variant, a BA.4/5 variant, a BA.4.6 variant, a BA.7 variant, a BQ.1 variant, a BQ.1.1 variant, a BA.2.75 variant, a BN.1 variant, a XBB variant, a XBB.1 variant, a XBB.1.5 variant, a BJ.1 variant, a BM.1.1.1 variant, a BA.2.3.20 variant, a BF.7 variant, a XBC variant, a CH.1.1 variant, a XBB.1.16 variant, a XBB.1.5.10 variant, a XBB.1.5.1 variant, a XBB.2.3 variant, a FL.1.5.1 variant, a XBB.1.5.CONV0817 variant, a XBB.1.5.70 variant, a BA.2.86 variant, a BA2.86 CONV1207 variant, a HV.1 variant, a HK.3 variant, a JN.1 variant, a JN.4 variant, a JD.1.1 variant, a BA.2 variant, a JN.1.11.1 variant, a GE.1.2.1 variant, a JN.1.13.1 variant, a KQ.1 variant, a KP.1.1 variant, a KP.3 variant, a KP.2 variant, a JN.1.50 variant, a KP.3.1.1 variant, a XEC variant, a LB.1 variant, a MV.1 variant, a LP.8.1 variant, a MC.10.2. variant, a LF.7 variant, a LP.8.1.2 variant, a FL.15.1.1 variant, or a LF.7.2.1 variant.

The antibodies are capable of binding to the spike protein of the coronavirus. CoV-S refers to the S protein of a coronavirus which is expressed on the surface of virions as a structural protein. As mentioned previously, the S protein plays an essential role for coronaviruses in binding to receptors on the host cell and determines host tropism (Zhu Z. et al., Infect Genet Evol. 2018 July; 61:183-184. doi: 10.1016/j.meegid.2018.03.028. Epub 2018 Apr. 4). SARS-CoV and SARS-CoV-2 bind to angiotensin-converting enzyme 2 (ACE2) of the host cell via the S protein's receptor-binding domains (RBDs) and uses ACE2 as a receptor to enter the host cells (Ge X. Y. et al., Nature. 2013 Nov. 28; 503(7477):535-8. doi: 10.1038/nature12711. Epub 2013 Oct. 30.; Hoffmann M. et al., Cell. 2020 Mar. 4. pii: S0092-8674(20)30229-4. doi: 10.1016/j.cell.2020.02.052). SARS-CoV can also use CD209L (also known as L-SIGN) as an alternative receptor (Jeffers S. A. et al., Proc Natl Acad Sci USA. 2004 Nov. 2; 101(44):15748-53. Epub 2004 Oct. 20). MERS-CoV binds dipeptidyl peptidase 4 (“DPP4”, also known as CD26) of the host cells via a different RBD of the S protein. Cell entry of coronaviruses depends on not only binding of the S protein to a host cell receptor but often also priming of the S protein by host cell proteases, and recently SARS-CoV-2 was found to use the serine protease TMPRSS2 for S protein priming and then ACE2 for entry (Wu A. et al., Cell Host Microbe. 2020 Mar. 11; 27(3):325-328. doi: 10.1016/j.chom.2020.02.001. Epub 2020 Feb. 7; Hoffmann M. et al., Cell. 2020 Mar. 4. pii: S0092-8674(20)30229-4. doi: 10.1016/j.cell.2020.02.052).

The S protein of SARS-CoV is referred to as SARS-CoV-S and may for example comprise the amino acid sequence of SEQ ID NO: 1 (1288 amino acids). The S protein of SARS-CoV-2 is referred to as SARS-CoV-2-S and may for example comprise the amino acid sequence of SEQ ID NO: 5 (1273 amino acids).

The present disclosure provides exemplary antibodies and antigen-binding antibody fragments that bind, e.g., specifically bind, to CoV, wherein at least some of these antibodies and antigen-binding antibody fragments bind to SARS-CoV-2-S and/or SARS-CoV-2-S. Due to the sequence similarity among different CoV species, such antibodies or antigen-binding antibody fragments of the present disclosure may also cross react with the S protein of other CoV species.

The exemplary S proteins of CoV that the antibodies or antigen-binding antibody fragments of the present disclosure may bind, e.g., specifically bind, include by way of example, Bat SARS CoV (GenBank Accession No. FJ211859), SARS CoV (GenBank Accession No. FJ211860), BtSARS.HKU3.1 (GenBank Accession No. DQ022305), BtSARS.HKU3.2 (GenBank Accession No. DQ084199), BtSARS.HKU3.3 (GenBank Accession No. DQ084200), BtSARS.Rml (GenBank Accession No. DQ412043), BtCoV.279.2005 (GenBank Accession No. DQ648857), BtSARS.Rfl (GenBank Accession No. DQ412042), BtCoV.273.2005 (GenBank Accession No. DQ648856), BtSARS.Rp3 (GenBank Accession No. DQ071615), SARS CoV.A022 (GenBank Accession No. AY686863), SARSCoV.CUHK-W1 (GenBank Accession No. AY278554), SARSCoV.GDO1 (GenBank Accession No. AY278489), SARSCoV.HC.SZ.61.03 (GenBank Accession No. AY515512), SARSCoV.SZ16 (GenBank Accession No. AY304488), SARSCoV.Urbani (GenBank Accession No. AY278741), SARSCoV.civet010 (GenBank Accession No. AY572035), or SARSCoV.MA.15 (GenBank Accession No. DQ497008), Rs SHCO14 (GenBank® Accession No. KC881005), Rs3367 (GenBank® Accession No. KC881006), WiV1 S (GenBank® Accession No. KC881007).

In some embodiments, the antibodies and antigen-binding antibody fragments provided herein may also bind to and neutralize existing bat CoV or pre-emergent bat CoVs. Antibodies and antigen-binding antibody fragments with such binding and/or neutralization abilities would be particularly useful in a future pandemic that may be caused by a spillover from an animal reservoir, like a bat.

Alternatively, the S proteins of CoV to which the antibodies or antigen-binding antibody fragments of the present disclosure may bind, e.g., specifically bind, to and neutralize pre-emergent coronaviruses from other species, e.g., bats.

Still alternatively, the S proteins of CoV to which the antibodies or antigen-binding antibody fragments of the present disclosure may bind, e.g., specifically bind, to may include, for example, Middle East respiratory syndrome coronavirus isolate Riyadh_2_2012 (GenBank Accession No. KF600652.1), Middle East respiratory syndrome coronavirus isolate Al-Hasa_18_2013 (GenBank Accession No. KF600651.1), Middle East respiratory syndrome coronavirus isolate Al-Hasa_17_2013 (GenBank Accession No. KF600647.1), Middle East respiratory syndrome coronavirus isolate Al-Hasa 15_2013 (GenBank Accession No. KF600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa_19_2013 (GenBank Accession No. KF600632), Middle East respiratory syndrome coronavirus isolate Buraidah_1_2013 (GenBank Accession No. KF600630.1), Middle East respiratory syndrome coronavirus isolate Hafr-Al-Batin_1_2013 (GenBank Accession No. KF600628.1), Middle East respiratory syndrome coronavirus isolate Al-Hasa_12_2013 (GenBank Accession No. KF600627.1), Middle East respiratory syndrome coronavirus isolate Bisha_1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate Riyadh_1_2012 (GenBank Accession No. KF600612.1), Middle East respiratory syndrome coronavirus isolate Al-Hasa_3_2013 (GenBank Accession No. KF186565.1), Middle East respiratory syndrome coronavirus isolate Al-Hasa_1_2013 (GenBank Accession No. KF186567.1), Middle East respiratory syndrome coronavirus isolate Al-Hasa_2_2013 (GenBank Accession No. KF186566.1), Middle East respiratory syndrome coronavirus isolate Al-Hasa_4_2013 (GenBank Accession No. KF186564.1), Middle East respiratory syndrome coronavirus (GenBank Accession No. KF192507.1), Betacoronavirus England 1-N1 (GenBank Accession No. NC_019843), MERS-CoV_SA-N1 (GenBank Accession No. KC667074), following isolates of Middle East Respiratory Syndrome Coronavirus (GenBank Accession No: KF600656.1, GenBank Accession No: KF600655.1, GenBank Accession No: KF600654.1, GenBank Accession No: KF600649.1, GenBank Accession No: KF600648.1, GenBank Accession No: KF600646.1, GenBank Accession No: KF600643.1, GenBank Accession No: KF600642.1, GenBank Accession No: KF600640.1, GenBank Accession No: KF600639.1, GenBank Accession No: KF600638.1, GenBank Accession No: KF600637.1, GenBank Accession No: KF600636.1, GenBank Accession No: KF600635.1, GenBank Accession No: KF600631.1, GenBank Accession No: KF600626.1, GenBank Accession No: KF600625.1, GenBank Accession No: KF600624.1, GenBank Accession No: KF600623.1, GenBank Accession No: KF600622.1, GenBank Accession No: KF600621.1, GenBank Accession No: KF600619.1, GenBank Accession No: KF600618.1, GenBank Accession No: KF600616.1, GenBank Accession No: KF600615.1, GenBank Accession No: KF600614.1, GenBank Accession No: KF600641.1, GenBank Accession No: KF600633.1, GenBank Accession No: KF600629.1, GenBank Accession No: KF600617.1), Coronavirus Neoromicia/PML-PHE1/RSA/2011 GenBank Accession: KC869678.2, Bat Coronavirus Taper/CII_KSA_287/Bisha/Saudi Arabia/GenBank Accession No: KF493885.1, Bat coronavirus Rhhar/CII_KSA_003/Bisha/Saudi Arabia/2013 GenBank Accession No: KF493888.1, Bat coronavirus Pikuh/CII_KSA_001/Riyadh/Saudi Arabia/2013 GenBank Accession No: KF493887.1, Bat coronavirus Rhhar/CII_KSA_002/Bisha/Saudi Arabia/2013 GenBank Accession No: KF493886.1, Bat Coronavirus Rhhar/CII_KSA_004/Bisha/Saudi Arabia/2013 GenBank Accession No: KF493884.1, BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV 133.2005 (GenBank Accession No. NC 008315), BtCoV.HKU5.5 (GenBank Accession No. EF065512); BtCoV.HKU5.1 (GenBank Accession No. NC_009020), BtCoV.HKU5.2 (GenBank Accession No. EF065510), BtCoV.HKU5.3 (GenBank Accession No. EF065511), human betacoronavirus 2c Jordan-N3/2012 (GenBank Accession No. KC776174.1; human betacoronavirus 2c EMC/2012 (GenBank Accession No. JX869059.2), Pipistrellus bat coronavirus HKU5 isolates (GenBank Accession No:KC522089.1, GenBank Accession No:KC522088.1, GenBank Accession No:KC522087.1, GenBank Accession No:KC522086.1, GenBank Accession No:KC522085.1, GenBank Accession No: KC522084.1, GenBank Accession No:KC522083.1, GenBank Accession No:KC522082.1, GenBank Accession No:KC522081.1, GenBank Accession No:KC522080.1, GenBank Accession No:KC522079.1, GenBank Accession No:KC522078.1, GenBank Accession No: KC522077.1, GenBank Accession No:KC522076.1, GenBank Accession No:KC522075.1, GenBank Accession No:KC522104.1, GenBank Accession No:KC522104.1, GenBank Accession No:KC522103.1, GenBank Accession No:KC522102.1, GenBank Accession No: KC522101.1, GenBank Accession No:KC522100.1, GenBank Accession No:KC522099.1, GenBank Accession No:KC522098.1, GenBank Accession No:KC522097.1, GenBank Accession No:KC522096.1, GenBank Accession No:KC522095.1, GenBank Accession No: KC522094.1, GenBank Accession No:KC522093.1, GenBank Accession No:KC522092.1, GenBank Accession No:KC522091.1, GenBank Accession No:KC522090.1, GenBank Accession No:KC522119.1 GenBank Accession No:KC522118.1 GenBank Accession No: KC522117.1 GenBank Accession No:KC522116.1 GenBank Accession No:KC522115.1 GenBank Accession No:KC522114.1 GenBank Accession No:KC522113.1 GenBank Accession No:KC522112.1 GenBank Accession No:KC522111.1 GenBank Accession No: KC522110.1 GenBank Accession No:KC522109.1 GenBank Accession No:KC522108.1, GenBank Accession No:KC522107.1, GenBank Accession No:KC522106.1, GenBank Accession No:KC522105.1) Pipistrellus bat coronavirus HKU4 isolates (GenBank Accession No:KC522048.1, GenBank Accession No:KC522047.1, GenBank Accession No:KC522046.1, GenBank Accession No:KC522045.1, GenBank Accession No: KC522044.1, GenBank Accession No:KC522043.1, GenBank Accession No:KC522042.1, GenBank Accession No:KC522041.1, GenBank Accession No:KC522040.1 GenBank Accession No:KC522039.1, GenBank Accession No:KC522038.1, GenBank Accession No:KC522037.1, GenBank Accession No:KC522036.1, GenBank Accession No:KC522048.1 GenBank Accession No:KC522047.1 GenBank Accession No:KC522046.1 GenBank Accession No:KC522045.1 GenBank Accession No:KC522044.1 GenBank Accession No:KC522043.1 GenBank Accession No:KC522042.1 GenBank Accession No:KC522041.1 GenBank Accession No:KC522040.1, GenBank Accession No:KC522039.1 GenBank Accession No:KC522038.1 GenBank Accession No:KC522037.1 GenBank Accession No:KC522036.1, GenBank Accession No:KC522061.1 GenBank Accession No:KC522060.1 GenBank Accession No:KC522059.1 GenBank Accession No:KC522058.1 GenBank Accession No:KC522057.1 GenBank Accession No:KC522056.1 GenBank Accession No:KC522055.1 GenBank Accession No:KC522054.1 GenBank Accession No:KC522053.1 GenBank Accession No:KC522052.1 GenBank Accession No:KC522051.1 GenBank Accession No:KC522050.1 GenBank Accession No:KC522049.1 GenBank Accession No:KC522074.1, GenBank Accession No:KC522073.1 GenBank Accession No:KC522072.1 GenBank Accession No:KC522071.1 GenBank Accession No:KC522070.1 GenBank Accession No:KC522069.1 GenBank Accession No:KC522068.1 GenBank Accession No:KC522067.1, GenBank Accession No:KC522066.1 GenBank Accession No:KC522065.1 GenBank Accession No:KC522064.1, GenBank Accession No:KC522063.1, or GenBank Accession No:KC522062.1.

Alternatively, the S proteins of CoV to which the antibodies or antigen-binding antibody fragments of the present disclosure may bind, e.g., specifically bind, may include for example, FCov.FIPV.79.1146.VR.2202 (GenBank Accession No. NV_007025), transmissible gastroenteritis virus (TGEV) (GenBank Accession No. NC_002306; GenBank Accession No. Q811789.2; GenBank Accession No. DQ811786.2; GenBank Accession No. DQ811788.1; GenBank Accession No. DQ811785.1; GenBank Accession No. X52157.1; GenBank Accession No. AJ011482.1; GenBank Accession No. KC962433.1; GenBank Accession No. AJ271965.2; GenBank Accession No. JQ693060.1; GenBank Accession No. KC609371.1; GenBank Accession No. JQ693060.1; GenBank Accession No. JQ693059.1; GenBank Accession No. JQ693058.1; GenBank Accession No. JQ693057.1; GenBank Accession No. JQ693052.1; GenBank Accession No. JQ693051.1; GenBank Accession No. JQ693050.1), or porcine reproductive and respiratory syndrome virus (PRRSV) (GenBank Accession No. NC_001961.1; GenBank Accession No. DQ811787).

Alternatively, the S proteins of CoV to which the antibodies or antigen-binding antibody fragments of the present disclosure may bind, e.g., specifically bind, may include, for example, BtCoV.1A.AFCD62 (GenBank Accession No. NC_010437), BtCoV.1B.AFCD307 (GenBank Accession No. NC_010436), BtCov.HKU8.AFCD77 (GenBank Accession No. NC_010438), BtCoV.512.2005 (GenBank Accession No. DQ648858), porcine epidemic diarrhea virus PEDV.CV777 (GenBank Accession No. NC_003436, GenBank Accession No. DQ355224.1, GenBank Accession No. DQ355223.1, GenBank Accession No. DQ355221.1, GenBank Accession No. JN601062.1, GenBank Accession No. N601061.1, GenBank Accession No. JN601060.1, GenBank Accession No. JN601059.1, GenBank Accession No. JN601058.1, GenBank Accession No. JN601057.1, GenBank Accession No. JN601056.1, GenBank Accession No. JN601055.1, GenBank Accession No. JN601054.1, GenBank Accession No. JN601053.1, GenBank Accession No. JN601052.1, GenBank Accession No. JN400902.1, GenBank Accession No. JN547395.1, GenBank Accession No. FJ687473.1, GenBank Accession No. FJ687472.1, GenBank Accession No. FJ687471.1, GenBank Accession No. FJ687470.1, GenBank Accession No. FJ687469.1, GenBank Accession No. FJ687468.1, GenBank Accession No. FJ687467.1, GenBank Accession No. FJ687466.1, GenBank Accession No. FJ687465.1, GenBank Accession No. FJ687464.1, GenBank Accession No. FJ687463.1, GenBank Accession No. FJ687462.1, GenBank Accession No. FJ687461.1, GenBank Accession No. FJ687460.1, GenBank Accession No. FJ687459.1, GenBank Accession No. FJ687458.1, GenBank Accession No. FJ687457.1, GenBank Accession No. FJ687456.1, GenBank Accession No. FJ687455.1, GenBank Accession No. FJ687454.1, GenBank Accession No. FJ687453 GenBank Accession No. FJ687452.1, GenBank Accession No. FJ687451.1, GenBank Accession No. FJ687450.1, GenBank Accession No. FJ687449.1, GenBank Accession No. AF500215.1, GenBank Accession No. KF476061.1, GenBank Accession No. KF476060.1, GenBank Accession No. KF476059.1, GenBank Accession No. KF476058.1, GenBank Accession No. KF476057.1, GenBank Accession No. KF476056.1, GenBank Accession No. KF476055.1, GenBank Accession No. KF476054.1, GenBank Accession No. KF476053.1, GenBank Accession No. KF476052.1, GenBank Accession No. KF476051.1, GenBank Accession No. KF476050.1, GenBank Accession No. KF476049.1, GenBank Accession No. KF476048.1, GenBank Accession No. KF177258.1, GenBank Accession No. KF177257.1, GenBank Accession No. KF177256.1, GenBank Accession No. KF177255.1), HCoV.229E (GenBank Accession No. NC_002645), HCoV.NL63.Amsterdam.I (GenBank Accession No. NC_005831), BtCoV.HKU2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), or BtCoV.HKU2.GD.430.2006 (GenBank Accession No. EF203064).

Alternatively, the S proteins of CoV to which the antibodies or antigen-binding antibody fragments of the present disclosure may bind, e.g., specifically bind, may include, for example, HCoV.HKU1.C.N5 (GenBank Accession No. DQ339101), MHV.A59 (GenBank Accession No. NC 001846), PHEV.VW572 (GenBank Accession No. NC 007732), HCoV.OC43.ATCC.VR.759 (GenBank Accession No. NC_005147), or bovine enteric coronavirus (BCoV.ENT) (GenBank Accession No. NC_003045).

Alternatively, the S proteins of CoV to which the antibodies or antigen-binding antibody fragments of the present disclosure may bind, e.g., specifically bind, may include, for example, BtCoV.HKU9.2 (GenBank Accession No. EF065514), BtCoV.HKU9.1 (GenBank Accession No. NC_009021), BtCoV.HkU9.3 (GenBank Accession No. EF065515), or BtCoV.HKU9.4 (GenBank Accession No. EF065516).sarbecovirus

In some instances, an anti-CoV-S antibody or antigen-binding fragment thereof according to the disclosure binds to CoV-S (e.g., SARS-CoV-S and/or SARS-CoV-2-S, and/or any of the CoV S proteins listed above) with a dissociation constant (KD) of (i) 100 nM or lower; (ii) about 10 nM or lower; (iii) about 1 nM or lower; (iv) about 100 pM or lower; (v) about 10 pM or lower; (vi) about 1 pM or lower; or (vii) about 0.1 pM or lower.

In some instances, an anti-CoV-S antibody or antigen-binding fragment thereof according to the disclosure binds to the CoV-S of the XBB.1.5 variant, the BQ.1 variant, the BA.2 variant, the BA.4/5 variant, or the BA.2.86 variant, with a dissociation constant (KD) of (i) 100 nM or lower; (ii) about 20 nM or lower; (iii) about 1 nM or lower; (iv) about 100 pM or lower; (v) about 10 pM or lower; (vi) about 1 pM or lower; or (vii) about 0.1 pM or lower.

The present disclosure provides exemplary antibodies or antigen-binding fragments thereof that bind CoV-S, including human CoV-S, which optionally may be affinity-matured. Other antibodies or antigen-binding fragments thereof that bind CoV-S, including those having different CDRs, and epitopic specificity may be obtained using the disclosure of the present specification, and using methods that are generally known in the art. Such antibodies and antigen-binding fragments thereof antagonize the biological effects of CoV-S in vivo and therefore are useful in treating or preventing COV-S-related conditions including, particularly coronavirus infection. In preferred embodiments, the antibody or antigen-binding fragment thereof according to the disclosure comprises one or more CDRs, a VL chain and/or VH chain of the anti-CoV-S antibodies and antigen-binding fragments thereof described herein.

In some embodiments, an anti-CoV-S antibody or antigen-binding fragment thereof according to the disclosure will interfere with, block, reduce, or modulate the interaction between COV-S and its receptor(s) (e.g., ACE2, CD209L, L-SIGN, DPP4, or CD26) on host cells or a S protein-priming protein on host cells (e.g., TMPRSS2). If binding of the S protein to its receptor is blocked or reduced, CoV virions may be prohibited from entering the cells, i.e., infection to further cells is prevented. Also, if the S protein is prevented from binding to a S protein-priming protein, the S protein would not be activated and therefore the host cell entry via the receptor may be reduced, i.e., infection to further cells is prevented.

In some instance, an anti-CoV-S antibody or antigen-binding fragment thereof according to the disclosure is “neutralizing”, e.g., it substantially or totally prevents the specific interaction of CoV-S with the host receptors or priming protein. As a result, CoV virions may be substantially or totally cleared by immune cells of the host, such as phagocytes via, for example, Fc receptor mediated phagocytosis or mere phagocytosis due to increased time of virions outside the cells. In some embodiments, the antibody or antigen-binding fragment thereof neutralizes CoV-S, e.g., by remaining bound to CoV-S in a location and/or manner that prevents CoV-S from binding to its receptor or priming protein on host cells. As a result, CoV virions may be substantially or totally prevented from entering the cells, i.e. infection to further cells is prevented.

In certain embodiments, an anti-CoV-S antibody or antigen-binding fragment thereof according to the disclosure neutralizes CoV (e.g., SARS-CoV and/or SARS-CoV-2) at an IC50 of about 100 nM or lower, of about 50 nM or lower, of about 20 nM or lower, of about 10 nM or lower, of about 5 nM or lower, of about 2 nM or lower, of about 1 nM or lower, of about 500 pM or lower, of about 200 pM or lower, of about 100 pM or lower, of about 50 pM or lower, of about 20 pM or lower, of about 10 pM or lower, of about 5 pM or lower, of about 2 pM or lower, or of about 1 pM or lower, or at an IC50 of about 500 ng/mL or lower, of about 200 ng/mL or lower, of about 100 ng/mL or lower, of about 50 ng/mL or lower, at about 20 ng/mL or lower, at about 10 ng/mL or lower, at about 5 ng/mL or lower, at about 2 ng/mL or lower, or at about 1 ng/mL or lower, in vitro, as measured by any of the neutralization assays described in Examples herein.

In some embodiments, the antibody, or antigen-binding fragment thereof, neutralizes the BA.1 variant, the XBB.1.5 variant, the XBB.1.16 variant, the XBB.1.5.10 variant, the XBB.1.5.1 variant, the XBB.2.3 variant, the FL.1.5.1 variant, the XBB.1.5.CONV0817 variant, the BA.2.86 variant, the HV.1 variant, the HK.3 variant, the JN.1 variant, the JN.4 variant, the JD.1.1 variant, the BA.2 variant, the GE.1.2.1 variant, the JN.1.13.1 variant, the KQ.1 variant, the KP.1.1 variant, the KP.3 variant, the KP.2 variant, the JN.1.50 variant, the KP.3.1.1 variant, the XEC variant, the LB.1 variant, the MV.1 variant, the LP.8.1 variant, the MC.10.2. variant, the LF.7 variant, the LP.8.1.2 variant, or the LF.7.2.1 variant of SARS-CoV-2 with an IC50 of about 100 ng/mL or lower, of about 50 ng/mL the FL.15.1.1 variant, or lower, of about 40 ng/mL or lower, of about 30 ng/mL or lower, of about 20 ng/mL or lower, of about 10 mg/mL or lower, of about 5 ng/mL or lower, of about 2 ng/mL or lower, or of about 1 ng/mL or lower, in vitro.

In some instances, an anti-CoV-S antibody or antigen-binding fragment thereof according to the disclosure or cocktail thereof, when administered to a coronavirus infected host or one susceptible to coronavirus infection such as a health care worker may promote a neutralization response in the host against the coronavirus which is sufficient to permit the host to be able to mount an effective cell-mediated immune response against the virus, e.g., T cell-mediated or cytokine-mediated immune response against the coronavirus and/or to be more responsive to other treatment methods such as drugs, antivirals or other biologics.

As mentioned, the anti-CoV-S antibodies or antigen-binding fragments thereof according to the disclosure have a variety of uses. For example, the subject antibodies and fragments can be useful in prophylactic or therapeutic applications, as well as diagnostically in binding assays. The subject anti-CoV-S antibodies or antigen-binding fragments thereof are useful for affinity purification of CoV-S, in particular human CoV-S or its ligands and in screening assays to identify other antagonists of CoV-S activity. Some of the antibodies or antigen-binding fragments thereof are useful for inhibiting binding of CoV-S to its receptor(s) (e.g., ACE2, CD209L, L-SIGN, DPP4, or CD26) on host cells or a S protein-priming protein on host cells (e.g., TMPRSS2) or inhibiting COV-S-mediated activities and/or biological effects.

As used herein, the term “one or more biological effects associated with CoV-S refers to any biological effect mediated, induced, or otherwise attributable to CoV-S, e.g., binding properties, functional properties, and other properties of biological significance. Non-limiting exemplary biological effects of CoV-S include CoV-S binding to its receptor(s) (e.g., ACE2, CD209L, L-SIGN, DPP4, or CD26) on host cells or a S protein-priming protein on host cells (e.g., TMPRSS2), activation of host cells for allowing virus entry, activation of immune cells as a result of the entry of CoV into the cell, e.g., via presentation of CoV antigen(s) on the host cells' MHC molecule, and resulting inflammation. The subject anti-CoV-S antibodies are capable of inhibiting one, a combination of, or all of these exemplary CoV-S biological activities. For example, the anti-CoV-S antibodies and antigen-binding fragments thereof provided herein may neutralize CoV virions or reduce the infectivity of CoV virions.

The antibody or antigen-binding fragment thereof according to the disclosure can be used in a variety of therapeutic applications. For example, in some embodiments the anti-CoV-S antibody or antigen-binding fragment thereof are useful for treating conditions associated with CoV-S, such as, but not limited to, symptoms associated with CoV infection. The CoV may be any CoV, including SARS-CoV, SARS-CoV-2, MERS-CoV, HCoV-HKU1, HCoV-OC43, HCoV-229E, and HCoV-NL63, and also may be any of the CoV species listed above herein. In some embodiments, the CoV infection is a chronic infection. In some embodiments, the CoV infection is an acute infection.

Specific examples of CoV infection-associated symptoms are fever, cough, dry cough, shortness of breath or difficulty of breath, fatigue, aches, runny nose, congestion, sore throat, conjunctivitis, chest pain, headache, muscle ache, chills, loss of smell, and loss of taste, and gastrointestinal symptoms including diarrhea. Complications and/or diseases/disorders associated with coronavirus infection may include, for example, bronchitis, pneumonia, respiratory failure, acute respiratory failure, organ failure, multi-organ system failure, pediatric inflammatory multisystem syndrome, acute respiratory distress syndrome (a severe lung condition that causes low oxygen in the blood and organs), blood clots, cardiac conditions, myocardial injury, myocarditis, heart failure, cardiac arrest, acute myocardial infarction, dysrhythmias, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, gastrointestinal complications, aspergillosis, increased susceptibility to infection with another virus or bacteria, and/or pregnancy-related complications. Certain diseases and conditions, such as high blood pressure, type 1 diabetes, liver disease, overweight, chronic lung diseases including cystic fibrosis, pulmonary fibrosis, and asthma, compromised immune system due to transplant, use of an immunosuppressant, or HIV infection, and brain and nervous system condition, may increase the risk of CoV infection-associated complications and diseases.

The subject anti-CoV-S antibodies and antigen-binding fragments thereof may be used alone or in association with other active agents or drugs, including other biologics, to treat any subject in which blocking, inhibiting, or neutralizing the in vivo effect of CoV-S or blocking or inhibiting the interaction of CoV-S and its receptor(s) (e.g., ACE2, CD209L, L-SIGN, DPP4, or CD26) on host cells or a S protein-priming protein on host cells (e.g., TMPRSS2), is therapeutically desirable.

The anti-CoV-S antibodies and antigen-binding fragments thereof comprising the disclosure have binding affinity for CoV-S, such as SARS-CoV-S or SARS-CoV-S2. Some antibodies of the present disclosure binds to SARS-CoV-S or SARS-CoV-S2 with a similar K_{D} (M), while some antibodies of the present disclosure bind to SARS-CoV-S with a lower K_{D} (M) (i.e., higher affinity) than to SARS-CoV-S2, and some antibodies of the present disclosure bind to SARS-CoV-S-2 with a lower K_{D} (M) (i.e., higher affinity) than to SARS-CoV-S.

Exemplary anti-CoV antibodies and antigen-binding fragments thereof according to the disclosure, and the specific CDRs thereof are identified in this section. For convenience, the exemplified antibody or antigen-binding fragment thereof, and corresponding sequences are separately identified by a specific nomenclature as shown in Table 13.

The anti-CoV-S antibodies and antigen-binding fragments thereof comprising the disclosure have binding affinity for CoV-S, such as SARS-CoV-S or SARS-CoV-S2. Some antibodies of the present disclosure binds to SARS-CoV-S or SARS-CoV-S2 with a similar K_{D} (M), while some antibodies of the present disclosure bind to SARS-CoV-S with a lower K_{D} (M) (i.e., higher affinity) than to SARS-CoV-S2, and some antibodies of the present disclosure bind to SARS-CoV-S-2 with a lower K_{D} (M) (i.e., higher affinity) than to SARS-CoV-S.

### C. Anti-CoV-S Antibody Polypeptide Sequences and Nucleic Acid Sequences Encoding Thereof

### Antibodies Disclosed Herein

Anti-CoV-S antibodies, and antigen-binding fragments thereof, specifically provided by the present disclosure include VYD2311, ADI-90031, ADI-90032, ADI-90033, ADI-90035, and antigen-binding fragments thereof. Any Fe variant may be used in combination with any of the variable sequences disclosed herein.

Table 13 shows (i) the amino acid sequences of the VH, VH FR1, VH CDR1, VH FR2, VH CDR2, VH FR3, VH CDR3, VH FR4, VL, VL FR1, VL CDR1, VL FR2, VL CDR2, VL FR3, VL CDR3, and VL FR4, and (ii) the DNA sequences of the VH and VL chains for the antibody.

### Variations of the Disclosed Antibodies and Polynucleotide Sequences Encoding Such Variations

In one embodiment, the disclosure contemplates anti-CoV-S antibodies or antigen-binding antibody fragments comprising (i) a VH CDR that is same as the VH CDR3 of, (ii) a VH CDR3 and VL CDR3, both of which as same as both of the VH CDR3 and the VL CDR3 of, (iii) at least 1, 2, 3, 4, 5, or 6 CDRs that are same as the corresponding CDR(s) of, or (iv) 6 CDRs that are all the same as the 6 CDRs of the antibody of the present disclosure, e.g., VYD2311, ADI-90031, ADI-90032, ADI-90033, ADI-90035; particularly VYD2311.

In some embodiments, the disclosure contemplates anti-CoV-S antibodies or antigen-binding antibody fragments, wherein (a) the VH comprises an amino acid sequence with at least 80, 85, 90, 91, 92, 93, 94, 95, 96, 97, 98, 99, or 100% sequence identity to the amino acid sequence of the VH of, and (b) the VL comprises an amino acid sequence with at least 80, 85, 90, 91, 92, 93, 94, 95, 96, 97, 98, 99, or 100% sequence identity to the amino acid sequence of the VL of the antibody of the present disclosure, e.g., VYD2311, ADI-90031, ADI-90032, ADI-90033, ADI-90035; particularly VYD2311.

In further embodiments, the disclosure contemplates anti-CoV-S antibodies or antigen-binding antibody fragments which optionally may be affinity-matured, comprising (i) a VH CDR that is same as the VH CDR3 of, (ii) a VH CDR3 and VL CDR3, both of which as same as both of the VH CDR3 and the VL CDR3 of, (iii) at least 1, 2, 3, 4, 5, or 6 CDRs that are same as the corresponding CDR(s) of, or (iv) 6 CDRs that are all the same as the 6 CDRs of the antibody of the present disclosure, e.g., VYD2311, ADI-90031, ADI-90032, ADI-90033, ADI-90035; particularly VYD2311.

In further embodiments, the disclosure contemplates anti-CoV-S antibodies or antigen-binding antibody fragments which optionally may be affinity-matured, comprising one of the CDR requirements (i)-(iv) of the immediately above paragraph, further wherein (a) the VH comprises an amino acid sequence with at least 80, 85, 90, 91, 92, 93, 94, 95, 96, 97, 98, 99, or 100% sequence identity to the amino acid sequence of the VH of, and (b) the VL comprises an amino acid sequence with at least 80, 85, 90, 91, 92, 93, 94, 95, 96, 97, 98, 99, or 100% sequence identity to the amino acid sequence of the VL of the antibody of the present disclosure, e.g., VYD2311, ADI-90031, ADI-90032, ADI-90033, ADI-90035; particularly VYD2311.

In other embodiments, the disclosure includes antibodies and antigen-binding fragments having binding specificity to CoV-S, which optionally may be affinity-matured, that bind the same epitope as the antibody of the present disclosure, e.g., VYD2311, ADI-90031, ADI-90032, ADI-90033, ADI-90035; particularly VYD2311.

In other embodiments, the anti-CoV-S antibodies and antigen-binding fragments of the disclosure which optionally may be affinity-matured, comprise, or alternatively consist of, combinations of one or more of the FRs, CDRs, the VH and VL sequences, and the heavy chain and light chain sequences set forth above, including all of them, or sequences that are at least 90%, 91%, 92%, 93%, 94%, 95%, 96%, 97%, 98%, or 99% identical thereto.

In a further embodiment of the disclosure, antigen-binding fragments comprise, or alternatively consist of, Fab fragments having binding specificity for CoV-S. The Fab fragment preferably includes the VH and the VL sequence of the antibody of the present disclosure, e.g., VYD2311, ADI-90031, ADI-90032, ADI-90033, ADI-90035; particularly VYD2311, or sequences that are at least 90%, 91%, 92%, 93%, 94%, 95%, 96%, 97%, 98%, or 99% identical thereto. This embodiment of the disclosure further includes Fabs containing additions, deletions, and variants of such VH and VL sequence while retaining binding specificity for CoV-S.

In some embodiments, the disclosure contemplates anti-CoV-S antibodies or antigen-binding antibody fragments, wherein (a) the VH comprises a VH CDR1 comprising the amino acid sequence FX₁FGSYEMN of SEQ ID NO: 151, wherein X₁ is E or D; a VH CDR2 comprising the amino acid sequence SISEDGX₁X₂TYYPDSLKG of SEQ ID NO: 152, wherein X₁ is Y or R, and X₂ is T or S; and a VH CDR3 comprising the amino acid sequence ARDFGGDTX₁WAGTGFTY of SEQ ID NO: 153, wherein X₁ is N or A; and the VL comprises a VL CDR1 comprising the amino acid sequence X₁GX₂SSNIGAGYDVH of SEQ ID NO: 154, wherein X₁ is E or T, and X₂ is S or T; a VL CDR2 comprising the amino acid sequence GSSX₁RNY of SEQ ID NO: 155, wherein X₁ is V, L, E or S; and a VL CDR3 comprising the amino acid sequence QSYDSDLGX₁LYT of SEQ ID NO: 156, wherein X₁ is I or V.

In some embodiments of the disclosure described herein, Fab fragments may be produced by enzymatic digestion (e.g., papain) of the parent full antibody. In another embodiment of the disclosure, anti-CoV-S antibodies such as the antibody of the present disclosure, e.g., VYD2311, ADI-90031, ADI-90032, ADI-90033, ADI-90035, and Fab fragments thereof may be produced via expression in mammalian cells, such as CHO, NS0, or HEK 293 cells, fungal, insect, or microbial systems, such as yeast cells.

In additional embodiments, the disclosure is further directed to polynucleotides encoding antibody polypeptides having binding specificity to CoV-S, including the VH and VL of the antibody of the present disclosure, e.g., VYD2311, ADI-90031, ADI-90032, ADI-90033, ADI-90035; particularly VYD2311, as well as fragments, variants, optionally affinity-matured variants, and combinations of one or more of the FRs, CDRs, the VH and VL sequences, and the heavy chain and light chain sequences set forth above, including all of them, or sequences that are at least 90%, 91%, 92%, 93%, 94%, 95%, 96%, 97%, 98%, or 99% identical thereto.

In other embodiments, the disclosure contemplates isolated anti-CoV-S antibodies and antigen binding fragments comprising (i) a VH which is same as the VH of the antibody of the present disclosure, e.g., VYD2311, ADI-90031, ADI-90032, ADI-90033, ADI-90035; particularly VYD2311; or (ii) a VL which is same as the VL of the antibody of the present disclosure, e.g., VYD2311, ADI-90031, ADI-90032, ADI-90033, ADI-90035; particularly VYD2311, or a variant thereof, wherein optionally one or more of the framework region residues (“FR residues”) and/or CDR residues in said VH or VL polypeptide has been substituted with another amino acid residue resulting in an anti-CoV-S antibody that binds, e.g., specifically binds, CoV-S.

In some embodiments, the antibody, or antigen-binding fragment thereof, have a Tₘ of at least about 62-70° C., about 63-70° C., or about 64-70° C. In some embodiments, the antibody, or antigen-binding fragment thereof, have a Tₘ of at least about 62° C., about 63° C., about 64° C., about 65° C., about 66° C., about 67° C., about 68° C., about 69° C., or about 70° C.

In some embodiments, the antibody, or antigen-binding fragment thereof, have a Tₒₙ of at least about 56-70° C., about 57-70° C., or about 58-70° C. In some embodiments, the antibody, or antigen-binding fragment thereof, have a Tₒₙ of at least about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C.

In some embodiments, the antibody, or antigen-binding fragment thereof, have improved thermal and colloidal stability in vitro, which can lead to an improved shelf life and/or half-life, and small dosing volumes.

In some embodiments, the antibody, or antigen-binding fragment thereof, has a serum half-life of about 40-200 days, about 45-20 days, about 50-160 days, about 50-140 days, about 40-130 days, about 45-130 days, about 50-100 days, about 60-100 days, about 60-90 days, about 50-90 days, about 50-80 days, about 40-70 days, about 60-80 days, about 55-75 days, about 65-95 days, about 70-90 days, about 90-100 days, about 70-80 days, about 80-130 days, about 100-150 days, about 110-140 days, about 120-140 days, about 120-160 days, about 130-170 days, about 140-180 days, about 150-190 days, or about 160-200 days. In some embodiments, the antibody, or antigen-binding fragment thereof, has a serum half-life of about 40, 45, 50, 55, 60, 65, 70, 71, 72, 73, 74, 75, 76, 77, 78, 79, 80, 81, 82, 83, 84, 85, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 165, 170, 175, 180, 185, 190, 195, or 200 days.

The disclosure also includes humanized, primatized and other chimeric forms of these antibodies. The chimeric and humanized antibodies may include an Fe derived from IgG1, IgG2, IgG3, or IgG4 constant regions.

In some embodiments of the disclosure, the chimeric or humanized antibodies or fragments or VH or VL polypeptides originate or are derived from one or more human antibodies, e.g., a human antibody identified from a clonal human B cell population.

In some aspects, the disclosure provides vectors comprising a nucleic acid molecule encoding an anti-CoV-S antibody or fragment thereof as disclosed herein. In some embodiments, the disclosure provides host cells comprising a nucleic acid molecule encoding an anti-CoV-S antibody or fragment thereof as disclosed herein.

In some aspects, the disclosure provides isolated antibodies or antigen binding fragments thereof that competes for binding to CoV-S with an antibody or antigen binding fragment thereof disclosed herein.

In some aspects, the disclosure provides a nucleic acid molecule, e.g., a DNA or an mRNA molecule, encoding any of the antibodies or antigen binding fragments disclosed herein.

In some aspects, the disclosure provides a pharmaceutical or diagnostic composition comprising at least one antibody or antigen binding fragment thereof as disclosed herein.

In some aspects, the disclosure provides a method for treating or preventing a condition associated with elevated CoV-S levels in a subject, comprising administering to a subject in need thereof an effective amount of at least one isolated antibody or antigen binding fragment thereof as disclosed herein.

In some aspects, the disclosure provides a method of inhibiting binding of CoV-S to its receptor (e.g., ACE2, L-SIGN, CD209L, DPP4, CD26) or an S protein-priming protein (e.g., TMPRSS2) in a subject comprising administering an effective amount of at least one antibody or antigen binding fragment thereof as disclosed herein. For example, administering VYD2311 may inhibit binding of CoV-S to its receptor, e.g., ACE2.

In some aspects, the disclosure provides an antibody or antigen binding fragment thereof that selectively binds to CoV-S, wherein the antibody or antigen binding fragment thereof binds to CoV-S with a K_{D} of less than or equal to 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, or 10⁻¹³ M; preferably, with a K_{D} of less than or equal to 5×10⁻¹⁰ M, 10⁻¹⁰ M, 5×10⁻¹¹ M, 10⁻¹¹ M, 5×10⁻¹² M, or 10⁻¹² M; more preferably, with a K_{D} that is less than about 100 pM, less than about 50 pM, less than about 40 pM, less than about 25 pM, less than about 1 pM, between about 10 pM and about 100 pM, between about 1 pM and about 100 pM, or between about 1 pM and about 10 pM. Preferably, the anti-CoV-S antibody or antigen binding fragment has cross-reactivity to the S protein of CoV other than SARS-CoV-S or SARS-CoV-2-S.

The inventive antibodies and antigen binding fragments thereof may be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Antibodies and antigen binding fragments thereof may also be chemically modified to provide additional advantages such as increased solubility, stability and circulating time (in vivo half-life) of the polypeptide, or decreased immunogenicity (See U.S. Pat. No. 4,179,337). The chemical moieties for derivatization may be selected from water soluble polymers such as polyethylene glycol, ethylene glycol/propylene glycol copolymers, carboxymethylcellulose, dextran, polyvinyl alcohol, and the like. The antibodies and fragments thereof may be modified at random positions within the molecule, or at predetermined positions within the molecule and may include one, two, three, or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. Branched polyethylene glycols are described, for example, in U.S. Pat. No. 5,643,575; Morpurgo et al., Appl. Biochem. Biotechnol., 56:59-72 (1996); Vorobjev et al., Nucleosides and Nucleotides, 18:2745-2750 (1999); and Caliceti et al., Bioconjug. Chem., 10:638-646 (1999), the disclosures of each of which are incorporated herein by reference.

There are a number of attachment methods available to those skilled in the art (See e.g., EP 0 401 384, herein incorporated by reference, disclosing a method of coupling PEG to G-CSF; and Malik et al., Exp. Hematol., 20:1028-1035 (1992) (reporting pegylation of GM-CSF using tresyl chloride)). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As described above, polyethylene glycol may be attached to proteins via linkage to any of a number of amino acid residues. For example, polyethylene glycol can be linked to polypeptides via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) or to more than one type of amino acid residue (e.g., lysine, histidine, aspartic acid, glutamic acid, cysteine and combinations thereof).

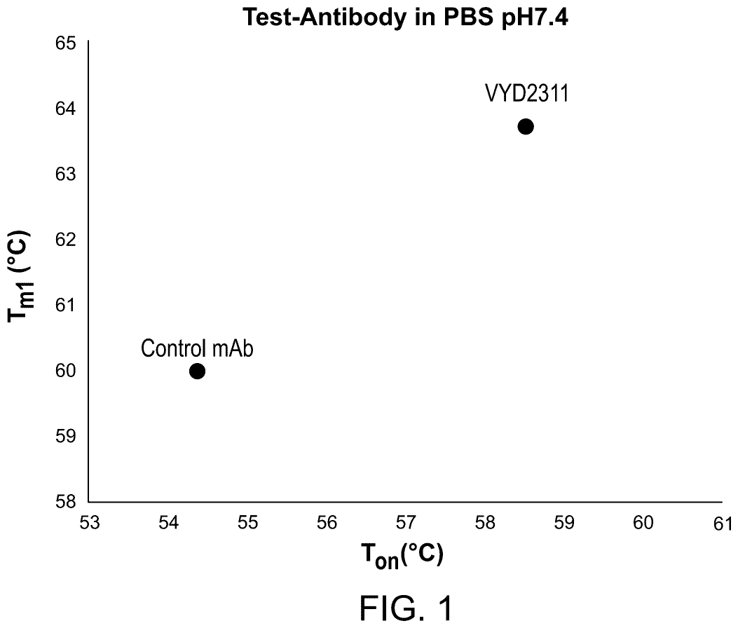

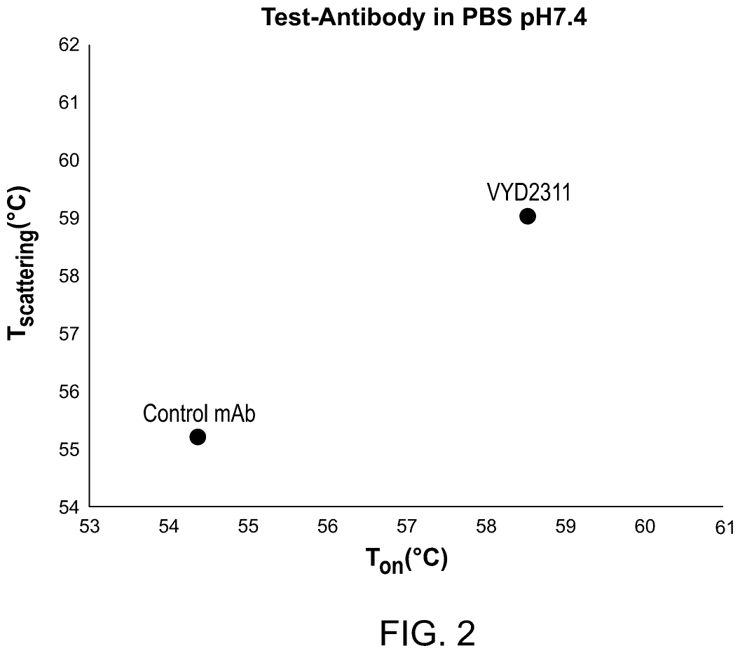

Alternatively, antibodies or antigen binding fragments thereof having increased in vivo half-lives may be produced via fusion with albumin (including but not limited to recombinant human serum albumin or fragments or variants thereof (See, e.g., U.S. Pat. No. 5,876,969, EP 0 413 622, and U.S. Pat. No. 5,766,883, herein incorporated by reference in their entirety)), or other circulating blood proteins such as transferrin or ferritin. In a preferred embodiment, polypeptides and/or antibodies of the present disclosure (including fragments or variants thereof) are fused with the mature form of human serum albumin (i.e., amino acids 1-585 of human serum albumin as shown in FIGS. 1 and 2 of EP 0 322 094) which is herein incorporated by reference in its entirety. Polynucleotides encoding fusion proteins of the disclosure are also encompassed by the disclosure.

Regarding detectable moieties, further exemplary enzymes include, but are not limited to, horseradish peroxidase, acetylcholinesterase, alkaline phosphatase, beta-galactosidase, and luciferase. Further exemplary fluorescent materials include, but are not limited to, rhodamine, fluorescein, fluorescein isothiocyanate, umbelliferone, dichlorotriazinylamine, phycoerythrin, and dansyl chloride. Further exemplary chemiluminescent moieties include, but are not limited to, luminol. Further exemplary bioluminescent materials include, but are not limited to, luciferin and aequorin. Further exemplary radioactive materials include, but are not limited to, Iodine 125 (¹²⁵I), Carbon 14 (¹⁴C), Sulfur 35 (³⁵S), Tritium (3H) and Phosphorus 32 (³²P).

Methods are known in the art for conjugating an antibody or antigen binding fragment thereof to a detectable moiety and the like, such as for example those methods described by Hunter et al., Nature, 144:945 (1962); David et al., Biochemistry, 13:1014 (1974); Pain et al., J. Immunol. Meth., 40:219 (1981); and Nygren, J., Histochem. and Cytochem., 30:407 (1982).

Embodiments described herein further include variants and equivalents that are substantially homologous to the antibodies, antibody fragments, diabodies, SMIPs, camelbodies, nanobodies, IgNAR, polypeptides, variable regions, and CDRs set forth herein. These may contain, e.g., conservative substitution mutations, (i.e., the substitution of one or more amino acids by similar amino acids). For example, conservative substitution refers to the substitution of an amino acid with another within the same general class, e.g., one acidic amino acid with another acidic amino acid, one basic amino acid with another basic amino acid, or one neutral amino acid by another neutral amino acid. The intent of a conservative amino acid substitution is well known in the art.

In other embodiments, the disclosure contemplates polypeptide sequences having at least 90% or greater sequence homology to any one or more of the polypeptide sequences of antigen binding fragments, variable regions and CDRs set forth herein. More preferably, the disclosure contemplates polypeptide sequences having at least 95% or greater sequence homology, even more preferably at least 98% or greater sequence homology, and still more preferably at least 99% or greater sequence homology to any one or more of the polypeptide sequences of antigen binding fragments, variable regions, and CDRs set forth herein.

Methods for determining homology between nucleic acid and amino acid sequences are well known to those of ordinary skill in the art.

In other embodiments, the disclosure further contemplates the above-recited polypeptide homologs of the antigen binding fragments, variable regions and CDRs set forth herein further having anti-CoV-S activity. Non-limiting examples of anti-CoV-S activity are set forth herein, e.g., ability to inhibit CoV-S binding to its receptor such as ACE2 or L-SIGN or an S protein-priming protein, thereby resulting in the reduced entry of CoV into cells.

In other embodiments, the disclosure further contemplates the generation and use of antibodies that bind any of the foregoing sequences, including, but not limited to, anti-idiotypic antibodies. In an exemplary embodiment, such an anti-idiotypic antibody could be administered to a subject who has received an anti-CoV-S antibody to modulate, reduce, or neutralize, the effect of the anti-CoV-S antibody. Such antibodies could also be useful for treatment of an autoimmune disease characterized by the presence of anti-CoV-S antibodies. A further exemplary use of such antibodies, e.g., anti-idiotypic antibodies, is for detection of the anti-CoV-S antibodies of the present disclosure, for example to monitor the levels of the anti-CoV-S antibodies present in a subject's blood or other bodily fluids. For example, in one embodiment, the disclosure provides a method of using the anti-idiotypic antibody to monitor the in vivo levels of said anti-CoV-S antibody or antigen binding fragment thereof in a subject or to neutralize said anti-CoV-S antibody in a subject being administered said anti-CoV-S antibody or antigen binding fragment thereof.

The present disclosure also contemplates anti-CoV-S antibodies comprising any of the polypeptide or polynucleotide sequences described herein substituted for any of the other polynucleotide sequences described herein. For example, without limitation thereto, the present disclosure contemplates antibodies comprising the combination of any of the VL and VH sequences described herein, and further contemplates antibodies resulting from substitution of any of the CDR sequences described herein for any of the other CDR sequences described herein.

Another embodiment of the disclosure contemplates these polynucleotides incorporated into an expression vector for expression in mammalian cells such as CHO, NS0, or HEK-293 cells, or in fungal, insect, or microbial systems such as yeast cells. In one embodiment of the disclosure described herein, Fab fragments can be produced by enzymatic digestion (e.g., papain) of the antibody of the present disclosure, e.g., VYD2311; following expression of the full-length polynucleotides in a suitable host. In another embodiment, anti-CoV-S antibodies, such as the antibody of the present disclosure, e.g., VYD2311, or Fab fragments thereof, can be produced via expression of the polynucleotides encoding VYD2311, in mammalian cells such as CHO, NS0, or HEK 293 cells, fungal, insect, or microbial systems such as yeast cells.

Host cells and vectors comprising said polynucleotides are also contemplated.

The disclosure further contemplates vectors comprising the polynucleotide sequences encoding the variable heavy and light chain polypeptide sequences, as well as the individual CDRs (hypervariable regions), as set forth herein, as well as host cells comprising said vector sequences. In embodiments of the disclosure, the host cells are mammalian cells, such as CHO cells. In embodiments of the disclosure, the host cells are yeast cells.

### D. Antibody-Drug Conjugate Comprising Anti-CoV-S Antibody

In some aspects, the disclosure is further directed to antibody-drug conjugates (ADCs) comprising (a) any antibody or antigen-binding antibody fragment described herein; and (b) a drug conjugated to the antibody or antigen-binding antibody fragment, either directly or indirectly (e.g., via a linker), and the use of antibody-drug conjugates for the methods of the present application.

In some aspects, the drug may be, but not limited to, a cytotoxic drug, an apoptotic drug, an immunostimulatory drug, an anti-microbial drug, an antibacterial drug or vaccine, an antiviral drug, antihelminth drug, antiparasitic drug, an anti-inflammatory drug, antihistamine, an anti-fibrotic drug, an immunosuppressive drug, a steroid, a bronchodilator, a beta blocker, an ACE inhibitor, an enzyme, a serine protease inhibitor, a toxin, a radioisotope, a compound, a small molecule, a small molecule inhibitor, a protein, a peptide, a vector, a plasmid, a viral particle, a nanoparticle, a DNA molecule, an RNA molecule, an siRNA, an shRNA, a micro RNA, an oligonucleotide, and an imaging drug.

An antiviral drug may be remdesivir, favipiravir, darunavir, nelfinavir, saquinavir, lopinavir, nirmatrelvir, or ritonavir; an antihelminth drug may be ivermectin; an antiparasite drug may be hydroxychloroquine, chloroquine, or atovaquone; antibacterial drug or vaccine may be the tuberculosis vaccine BCG; an anti-inflammatory drug, may be ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; an antihistamine drug may be bepotastine; an ACE inhibitor may be moexipril; and a drug that inhibits priming of CoV-S may be a serine protease inhibitor such as nafamostat.

The toxin may be a bacterial, fungal, plant, or animal toxin, or a fragment thereof. Examples include, but are not limited to, diphtheria A chain, diphtheria toxin, exotoxin A chain, ricin A chain, abrin A chain, modeccin A chain, alpha sarcin, Aleurites fordii protein, a dianthin protein, or a Phytolacca Americana protein.

The a cytotoxic drug or anti-proliferative drug may be, for example, but is not limited to, doxorubicin, daunorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, epothilone, or a derivative thereof (for example, see Polakis P. et al., Pharmacol Rev. 2016 January; 68(1):3-19. doi: 10.1124/pr.114.009373) (the drugs may be obtained from many vendors, including Creative Biolabs®).

The radioisotope may be for example, but is not limited to, At²¹¹, I¹³¹, In¹³¹, I²⁵, Y⁹⁰, Re¹⁸⁶, Re¹⁸⁸, Sm¹⁵³, Bi²¹², P³², Pb²¹² and radioactive isotopes of Lu.

In certain embodiments, the drug may be, but is not limited to, MMAE or MMAF.

In some embodiments, the Ab or antigen-binding Ab fragment is directly conjugated to the drug to form an ADC.

In some embodiments, the antibody or antigen-binding antibody fragment is indirectly conjugated to the drug to form an ADC.

Any appropriate conjugation method may be used to generate an ADC (for example, Nolting B. Methods Mol Biol. 2013; 1045:71-100. doi: 10.1007/978-1-62703-541-5_5; Jain N. et al., Pharm Res. 2015 November; 32(11):3526-40. doi: 10.1007/s11095-015-1657-7. Epub 2015 Mar. 11; Tsuchikama K. et al., Protein Cell. 2018 January; 9(1):33-46. doi: 10.1007/s13238-016-0323-0. Epub 2016 Oct. 14; Polakis P. et al., Pharmacol Rev. 2016 January; 68(1):3-19. doi: 10.1124/pr.114.009373). Examples of methods that may be used to perform conjugation include, but are not limited to, chemical conjugation and enzymatic conjugation.

Chemical conjugation may utilize, for example, but is not limited to, lysine amide coupling, cysteine coupling, and/or non-natural amino acid incorporation by genetic engineering. Enzymatic conjugation may utilize, for example, but is not limited to, transpeptidation using sortase, transpeptidation using microbial transglutaminase, and/or N-Glycan engineering.

In certain aspects, one or more of cleavable linkers may be used for conjugation. The cleavable linker may enable cleavage of the drug upon responding to, for example, but not limited to, an environmental difference between the extracellular and intracellular environments (pH, redox potential, etc.) or by specific lysosomal enzymes.

Examples of the cleavable linker include, but are not limited to, hydrazone linkers, peptide linkers including cathepsin B-responsive linkers, such as valine-citrulline (vc) linker, disulfide linkers such as N-succinimidyl-4-(2-pyridyldithio) (SPP) linker or N-succinimidyl-4-(2-pyridyldithio)butanoate (SPDB) linker, and pyrophosphate diester linkers.

Alternatively or simultaneously, one or more of non-cleavable linkers may be used. Examples of non-cleavable linkers include thioether linkers, such as N-succinimidyl 4-(N-maleimidomethyl) cyclohexane-1-carboxylate (SMCC), and maleimidocaproyl (me) linkers. Generally, non-cleavable linkers are more resistant to proteolytic degradation and more stable compared to cleavable linkers.

### E. Chimeric Antigen Receptor Comprising Anti-CoV-S Antigen-Binding Antibody Fragment

The present application further provides the use of chimeric antigen receptor comprising the anti-CoV-S antigen-binding fragment for the methods of the disclosure. In some embodiments, a compound specific to CoV-S according to the present disclosure may be a chimeric antigen receptor (CAR). In particular, the CARs of the present disclosure comprise an antigen binding (AB) domain that binds to CoV-S, a transmembrane (TM) domain, and an intracellular signaling (ICS) domain.

In some embodiments, a CAR may comprise a hinge that joins the AB domain and said TM domain.

In some embodiments, the CAR may comprise one or more costimulatory (CS) domains.

### AB Domain

A CAR according to the disclosure will comprise an antigen-binding (AB) domain which binds to COV-S. In some embodiments, the AB domain of the CAR may comprise any of the anti-COV-S antigen-binding antibody fragments disclosed herein.

In some embodiments, the AB domain of the CAR may comprise any of the antigen-binding domain of any of the anti-COV-S antibodies disclosed herein.

In some embodiments, the AB domain of the CAR may comprise any of the anti-COV-S antibodies, anti-COV-S antigen-binding antibody fragments, anti-COV-S multi-specific Abs, anti-COV-S multi-specific antigen-binding antibody fragments, and anti-COV-S ADCs disclosed herein, or the ABD thereof.

In some embodiments, the AB domain of the CAR may comprise an anti-COV-S scFv.

In some embodiments, the AB domain may comprise an amino acid sequence at least 80%, at least 85%, at least 90%, at least 91%, at least 92%, at least 93%, at least 94%, at least 95%, at least 96%, at least 97%, at least 98%, at least 99%, or 100% identical to an scFv comprising the VH and VL of the antibody of the present disclosure, e.g., VYD2311.

In some aspects, the AB domain may compete for binding to CoV-S with the antibody of the present disclosure, e.g., VYD2311.

### Hinge

In some embodiments, the CAR may comprise a hinge sequence between the AB domain and the TM domain. One of the ordinary skill in the art will appreciate that a hinge sequence is a short sequence of amino acids that facilitates flexibility (see, e.g. Woof J. M. et al., Nat. Rev. Immunol., 4(2): 89-99 (2004)). The hinge sequence can be any suitable sequence derived or obtained from any suitable molecule.

In some embodiments, the length of the hinge sequence may be optimized based on the desired length of the extracellular portion of the CAR, which may be based on the location of the epitope within the target molecule. For example, if the epitope is in the membrane proximal region within the target molecule, longer hinges may be optimal.

In some embodiments, the hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the hinge includes at least a portion of an IgG1, an IgG2, an IgG3, an IgG4, an IgE, an IgM, or an IgA immunoglobulin Fc region that falls within its CH2 and CH3 domains. In some embodiments, the hinge may also include at least a portion of a corresponding immunoglobulin hinge region. In some embodiments, the hinge is derived from or includes at least a portion of a modified immunoglobulin Fc region, for example, a modified IgG1 Fc region, a modified IgG2 Fc region, a modified IgG3 Fc region, a modified IgG4 Fc region, a modified IgE Fc region, a modified IgM Fc region, or a modified IgA Fc region. The modified immunoglobulin Fc region may have one or more mutations (e.g., point mutations, insertions, deletions, duplications) resulting in one or more amino acid substitutions, modifications, or deletions that cause impaired binding of the hinge to an Fc receptor (FcR). In some aspects, the modified immunoglobulin Fc region may be designed with one or more mutations which result in one or more amino acid substitutions, modifications, or deletions that cause impaired binding of the hinge to one or more FcR including, but not limited to, FcγRI, FcγR2A, FcγR2B1, Fcγ2B2, Fcγ 3A, Fcγ 3B, FcεRI, FcεR2, FcαRI, Fca/μR, or FcRn.

In some aspects, a portion of the immunoglobulin constant region may serve as a hinge between the AB domain, for example scFv or nanobody, and the TM domain. The hinge can be of a length that provides for increased responsiveness of the CAR-expressing cell following antigen binding, as compared to in the absence of the hinge. In some examples, the hinge is at or about 12 amino acids in length or is no more than 12 amino acids in length. Exemplary hinges include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a hinge has about 12 amino acids or less, about 119 amino acids or less, or about 229 amino acids or less. Exemplary hinges include a CD28 hinge, IgG4 hinge alone, IgG4 hinge linked to CH2 and CH3 domains, or IgG4 hinge linked to the CH3 domain. Exemplary hinges include, but are not limited to, those described in Hudecek M. et al. (2013) Clin. Cancer Res., 19:3153, international patent application publication number WO2014031687, U.S. Pat. No. 8,822,647 or published App. No. US2014/0271635. Known hinge sequences include those derived from CD8 α molecule or a CD28 molecule.

### Transmembrane (TM) Domain

With respect to the TM domain, the CAR can be designed to comprise a TM domain that is fused to the AB domain of the CAR. A hinge sequence may be inserted between the AB domain and the TM domain. TM domains may be derived from a natural or from synthetic sources. Where the source is natural, the domain may be derived from any membrane-bound or transmembrane protein. Typically, a TM domain denotes a single transmembrane α helix of a transmembrane protein, also known as an integral protein. TM domains e.g., may be derived from (i.e. comprise at least the transmembrane region(s) of) CD28, CD3 ε, CD4, CD5, CD8, CD9, CD16, CD22, CD33, CD37, CD45, CD64, CD80, CD86, CD134, CD137, CD154, TCR α, TCR β, or CD3 zeta and/or TM domains containing functional variants thereof such as those retaining a substantial portion of the structural, e.g., transmembrane, properties thereof.

Alternatively, the TM domain may be synthetic, in which case the TM domain will comprise predominantly hydrophobic residues such as leucine and valine. Preferably a triplet of phenylalanine, tryptophan and valine will be found at each end of a synthetic TM domain. A TM domain is generally thermodynamically stable in a membrane. It may be a single α helix, a transmembrane β barrel, a β-helix of gramicidin A, or any other structure. Transmembrane helices are usually about 20 amino acids in length.

A well-used TM domain comprises the TM region of CD28, e.g., human CD28. Often, a short oligo- or polypeptide spacer, e.g., between 2 and 10 amino acids in length is used to form the linkage between the TM domain and the ICS domain(s) of the CAR.

### Intracellular Signaling (ICS) Domain and Costimulatory (CS) Domain

The ICS domain or the cytoplasmic domain of a CAR generally triggers or elicits activation of at least one of the normal effector functions of the cell in which the CAR has been placed. The term “effector function” refers to a specialized function of a cell. Effector function of a T cell, for example, may be cytolytic activity or helper activity including the secretion of cytokines. Thus, the term “intracellular signaling domain” or “ICS domain” refers to the portion of a protein which transduces the effector function signal and directs the cell to perform a specialized function. While usually the entire ICS domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling domain” or “ICS domain” is thus meant to include any truncated portion of the ICS domain sufficient to transduce the effector function signal.

Examples of known ICS domains include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Signals generated through one ICS domain alone may be insufficient for full activation of a cell, and a secondary or costimulatory signal may also be required. In such cases, a costimulatory domain (CS domain) may be included in the cytoplasmic portion of a CAR. A CS domain is a domain that transduces such a secondary or costimulatory signal. In some instances, a CAR of the present disclosure may comprise two or more CS domains. The CS domain(s) may be placed upstream of the ICS domain or downstream of the ICS domain.

T cell activation can be mediated by two distinct classes of cytoplasmic signaling sequence: those that initiate antigen-dependent primary activation through the TCR (primary cytoplasmic signaling sequences) and those that act in an antigen-independent manner to provide a secondary or costimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Such a cytoplasmic signaling sequence may be contained in the ICS or the CS domain of a CAR of the present disclosure.

Examples of ITAM-containing primary cytoplasmic signaling sequences include those derived from an ICS domain of a lymphocyte receptor chain, a TCR/CD3 complex protein, an Fc receptor subunit, an IL-2 receptor subunit, CDζ, FcR γ, FcRβ, CD3γ, CD3δ, CD3ε, CD5, CD22, CD66d, CD79a, CD79b, CD278 (ICOS), Fε RI, DAP10, and DAP12. A well-used ICS domain comprises a cytoplasmic signaling sequence derived from CD3 zeta. In some instances, the CD3ζ ICS domain may be combined with one or more of other cytoplasmic domain(s). For example, the cytoplasmic domain of the CAR can comprise a CD3ζ ICS domain and a CS domain wherein a CS region refers to a portion of the CAR comprising the intracellular domain of a costimulatory molecule. A costimulatory molecule is a cell surface molecule other than an antigen receptor or their ligands that is required for an efficient response of lymphocytes to an antigen.

Examples of co-stimulatory molecules include an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8 α, CD8 β, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, CRTAM, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R β, IL2R γ, IL7R α, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, a ligand that specifically binds with CD83, and the like. The ICS domain and the CS domain(s) of the CAR may be linked to each other in a random or specified order, optionally via a short oligo- or polypeptide linker, e.g., between 2 and 10 amino acids in length.

### Exemplary CAR Constructs

A CAR construct may comprise the following format: “AB domain-hinge-TM domain-CS domain-ICS domain.”

CARs of the present disclosure may comprise an amino acid sequence at least 80%, at least 85%, at least 90%, at least 91%, at least 92%, at least 93%, at least 94%, at least 95%, at least 96%, at least 97%, at least 98%, at least 99%, or 100% identical to any of the exemplary constructs below. In the exemplary constructs below, the “anti-CoV-S scFv” may be an scFv generated by linking the VH and VL (in the order of VH-linker-VL or VL-linker-VH) of the antibody of the present disclosure, e.g., VYD2311.

In some embodiments, a leader sequence (LS) may be placed upstream of the polynucleotide sequences encoding the CAR. The leader sequence facilitates expression of the CAR on the cell surface.

### Further Modification

CARs according to the present disclosure, nucleotide sequences encoding the same, vectors encoding the same, and cells comprising nucleotide sequences encoding said CARs may be further modified, engineered, optimized, or appended in order to provide or select for various features. These features may include, but are not limited to, efficacy, persistence, target specificity, reduced immunogenicity, multi-targeting, enhanced immune response, expansion, growth, reduced off-target effect, reduced subject toxicity, improved target cytotoxicity, improved attraction of disease alleviating immune cells, detection, selection, targeting, and the like. For example, the cells may be engineered to express another CAR, or to have a suicide mechanism, and may be modified to remove or modify expression of an endogenous receptor or molecule such as a TCR and/or MHC molecule.

In some embodiments, the vector or nucleic acid sequence encoding the CAR further encodes other genes. The vector or nucleic acid sequence may be constructed to allow for the co-expression of multiple genes using a multitude of techniques including co-transfection of two or more plasmids, the use of multiple or bidirectional promoters, or the creation of bicistronic or multicistronic vectors. The construction of multicistronic vectors may include the encoding of IRES elements or 2A peptides, such as T2A, P2A, E2A, or F2A (for example, see Kim, J. H., et al., “High cleavage efficiency of a 2A peptide derived from porcine teschovirus-1 in human cell lines, zebrafish and mice”, PLoS One. 2011; 6(4)). The CAR expressing cell may further comprise a disruption to one or more endogenous genes.

### Efficacy

The CARs of the present disclosure and cells expressing these CARs may be further modified to improve efficacy against cells expressing the target molecule. The cells may be cells expressing COV-S. The cells expressing COV-S may be cancer cells, vascular cells, or any other target disease-associated cells. In some embodiments, the improved efficacy may be measured by increased cytotoxicity against cells expressing the target molecule, for example cytotoxicity against cancer cells. In some embodiments, the improved efficacy may also be measured by increased production of cytotoxic mediators such as, but not limited to, IFN γ, perforin, and granzyme B. In some embodiments, the improved efficacy may be shown by reduction in the signature cytokines of the diseases, or alleviated symptoms of the disease when the CAR expressing cells are administered to a subject. Other cytokines that may be reduced include TGF-beta, IL-6, IL-4, IL-10, and/or IL-13. the improved efficacy may be shown by COV-S-specific immune cell responses, such as T cell cytotoxicity. In case of cancer, improved efficacy may be shown by better tumor cytotoxicity, better infiltration into the tumor, reduction of immunosuppressive mediators, reduction in weight decrease, reduction in ascites, reduction in tumor burden, and/or increased lifespan. In case of autoimmune diseases, reduced responsiveness of autoreactive cells or decrease in autoreactive T cells, B cells, or Abs may represent improved efficacy. In some embodiments, gene expression profiles may be also investigated to evaluate the efficacy of the CAR.

In one aspect, the CAR expressing cells are further modified to evade or neutralize the activity of immunosuppressive mediators, including, but not limited to prostaglandin E2 (PGE2) and adenosine. In some embodiments, this evasion or neutralization is direct. In other embodiments, this evasion or neutralization is mediated via the inhibition of protein kinase A (PKA) with one or more binding partners, for example ezrin. In a specific embodiment, the CAR-expressing cells further express the peptide “regulatory subunit I anchoring disruptor” (RIAD). RIAD is thought to inhibit the association of protein kinase A (PKA) with ezrin, which thus prevents PKA's inhibition of TCR activation (Newick K. et al. Cancer Immunol Res. 2016 June; 4(6):541-51. doi: 10.1158/2326-6066.CIR-15-0263. Epub 2016 Apr. 4).

In some embodiments, the CAR expressing cells of the disclosure may induce a broad immune response, consistent with epitope spreading.

In some embodiments, the CAR expressing cells of the disclosure further comprise a homing mechanism. For example, the cell may transgenically express one or more stimulatory chemokines or cytokines or receptors thereof. In particular embodiments, the cells are genetically modified to express one or more stimulatory cytokines. In certain embodiments, one or more homing mechanisms are used to assist the inventive cells to accumulate more effectively to the disease site. In some embodiments, the CAR expressing cells are further modified to release inducible cytokines upon CAR activation, e.g., to attract or activate innate immune cells to a targeted cell (so-called fourth generation CARs or TRUCKS). In some embodiments, CARs may co-express homing molecules, e.g., CCR4 or CCR2b, to increase trafficking to the disease site.

### Controlling CAR Expression

In some instances, it may be advantageous to regulate the activity of the CAR or CAR expressing cells CAR. For example, inducing apoptosis using, e.g., a caspase fused to a dimerization domain (see, e.g., Di et al., N Engl. J. Med. 2011 Nov. 3; 365(18):1673-1683), can be used as a safety switch in the CAR therapy of the instant disclosure. In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di et al. N. Engl. J. Med. 2011; 365:1673-83.

Alternative strategies for regulating the CAR therapy of the instant disclosure include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or compliment-induced cell death. For example, CAR expressing cells described herein may also express a receptor capable of being targeted by an antibody or antibody fragment. Examples of such receptors include EpCAM, VEGFR, integrins (e.g., integrins αvβ, α4, αI3/4β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain). For example, CAR-expressing cells described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., “Gene Ther. 2013; 20(8)853-860).

In some embodiments, the CAR cell comprises a polynucleotide encoding a suicide polypeptide, such as for example RQR8. See, e.g., WO2013153391A, which is hereby incorporated by reference in its entirety. In CAR cells comprising the polynucleotide, the suicide polypeptide may be expressed at the surface of a CAR cell. The suicide polypeptide may also comprise a signal peptide at the amino terminus. Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood 2014; 124(8)1277-1287). Other methods for depleting CAR-expressing cells include administration of CAMPATH®, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In some embodiments, a regulatable CAR (RCAR) where the CAR activity can be controlled is desirable to optimize the safety and efficacy of a CAR therapy. In some embodiments, a RCAR comprises a set of polypeptides, typically two in the simplest embodiments, in which the components of a standard CAR described herein, e.g., an AB domain and an ICS domain, are partitioned on separate polypeptides or members. In some embodiments, the set of polypeptides include a dimerization switch that, upon the presence of a dimerization molecule, can couple the polypeptides to one another, e.g., can couple an AB domain to an ICS domain. Additional description and exemplary configurations of such regulatable CARs are provided herein and in International Publication No. WO 2015/090229, hereby incorporated by reference in its entirety.

In an aspect, an RCAR comprises two polypeptides or members: 1) an intracellular signaling member comprising an ICS domain, e.g., a primary ICS domain described herein, and a first switch domain; 2) an antigen binding member comprising an AB domain, e.g., that binds, e.g., specifically binds, a target molecule described herein, as described herein and a second switch domain. Optionally, the RCAR comprises a TM domain described herein. In an embodiment, a TM domain can be disposed on the intracellular signaling member, on the antigen binding member, or on both. Unless otherwise indicated, when members or elements of an RCAR are described herein, the order can be as provided, but other orders are included as well. In other words, in an embodiment, the order is as set out in the text, but in other embodiments, the order can be different. E.g., the order of elements on one side of a transmembrane region can be different from the example, e.g., the placement of a switch domain relative to an ICS domain can be different, e.g., reversed.

In some embodiments, the CAR expressing immune cell may only transiently express a CAR. For example, the cells of the disclosure may be transduced with mRNA comprising a nucleic acid sequence encoding an inventive CAR. In this vein, the present disclosure also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequences (“UTRs”), a 5′ cap and/or Internal Ribosome Entry Site (IRES), the nucleic acid to be expressed, and a polyA tail, typically 50-2000 bases in length. RNA so produced can efficiently transfect different kinds of cells. In one embodiment, the template includes sequences for the CAR. In an embodiment, an RNA CAR vector is transduced into a cell by electroporation.

### Target Specificity

The CAR expressing cells of the present disclosure may further comprise one or more CARs, in addition to the first CAR. These additional CARs may or may not be specific for the target molecule of the first CAR. In some embodiments, the one or more additional CARs may act as inhibitory or activating CARs. In some aspects, the CAR of some embodiments is the stimulatory or activating CAR; in other aspects, it is the costimulatory CAR. In some embodiments, the cells further include inhibitory CARs (iCARs, see Fedorov et al., Sci. Transl. Medicine, 2013 December; 5(215): 215ra172), such as a CAR recognizing an antigen other than the target molecule of the first CAR, whereby an activating signal delivered through the first CAR is diminished or inhibited by binding of the inhibitory CAR to its ligand, e.g., to reduce off-target effects.

In some embodiments, the AB domain of the CAR is or is part of an immunoconjugate, in which the AB domain is conjugated to one or more heterologous molecule(s), such as, but not limited to, a cytotoxic agent, an imaging agent, a detectable moiety, a multimerization domain, or other heterologous molecule. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; antibiotics; toxins such as small molecule toxins or enzymatically active toxins. In some embodiments, the AB domain is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, to enhance persistence, the cells of the disclosure may be further modified to overexpress pro-survival signals, reverse anti-survival signals, overexpress Bel-xL, overexpress hTERT, lack Fas, or express a TGF-β dominant negative receptor. Persistence may also be facilitated by the administration of cytokines, e.g., IL-2, IL-7, and IL-15.

### F. B-cell Screening and Isolation

In one embodiment, the present disclosure contemplates the preparation and isolation of a clonal population of antigen-specific B-cells that may be used for isolating at least one CoV-S antigen-specific cell, which can be used to produce a monoclonal antibody against CoV-S, which is specific to a desired CoV-S antigen, or a nucleic acid sequence corresponding to such an antibody. Methods of preparing and isolating said clonal population of antigen-specific B-cells are taught, for example, in U.S. Patent Publication No. US2007/0269868 to Carvalho-Jensen et al., the disclosure of which is herein incorporated by reference in its entirety. Methods of preparing and isolating said clonal population of antigen-specific B-cells are also taught herein in the examples. Methods of “enriching” a cell population by size or density are known in the art. See, e.g., U.S. Pat. No. 5,627,052. These steps can be used in addition to enriching the cell population by antigen-specificity.

### G. Methods of Producing Antibodies and Fragments Thereof

In another embodiment, the present disclosure contemplates methods for producing anti-CoV-S antibodies and fragments thereof. Methods of producing antibodies are well known to those of ordinary skill in the art. For example, methods of producing chimeric antibodies are now well known in the art (See, for example, U.S. Pat. No. 4,816,567 to Cabilly et al.; Morrison et al., Proc. Natl. Acad. Sci. U.S.A., 81:8651-55 (1984); Neuberger et al., Nature, 314:268-270 (1985); Boulianne, G. L. et al., Nature, 312:643-46 (1984), the disclosures of each of which are herein incorporated by reference in their entireties).

As mentioned above, methods of producing humanized antibodies are now well known in the art (See, for example, U.S. Pat. Nos. 5,530,101, 5,585,089, 5,693,762, and 6,180,370 to Queen et al; U.S. Pat. Nos. 5,225,539 and 6,548,640 to Winter; U.S. Pat. Nos. 6,054,297, 6,407,213 and 6,639,055 to Carter et al; U.S. Pat. No. 6,632,927 to Adair; Jones, P. T. et al., Nature, 321:522-525 (1986); Reichmann, L. et al., Nature, 332:323-327 (1988); Verhoeyen, M. et al., Science, 239:1534-36 (1988), the disclosures of each of which are herein incorporated by reference in their entireties).

Antibody polypeptides of the disclosure having CoV-S binding specificity may also be produced by constructing, using conventional techniques well known to those of ordinary skill in the art, an expression vector containing a promoter (optionally as a component of a eukaryotic or prokaryotic operon) and a DNA sequence encoding an antibody heavy chain in which the DNA sequence encoding the CDRs required for antibody specificity is derived from a non-human cell source, e.g., a rabbit or rodent B-cell source, while the DNA sequence encoding the remaining parts of the antibody chain is derived from a human cell source.

A second expression vector is produced using the same conventional means well known to those of ordinary skill in the art, said expression vector containing a promoter (optionally as a component of a eukaryotic or prokaryotic operon) and a DNA sequence encoding an antibody light chain in which the DNA sequence encoding the CDRs required for antibody specificity is derived from a non-human cell source, e.g., a rabbit or rodent B-cell source, while the DNA sequence encoding the remaining parts of the antibody chain is derived from a human cell source.

The expression vectors are transfected into a host cell by convention techniques well known to those of ordinary skill in the art to produce a transfected host cell, said transfected host cell cultured by conventional techniques well known to those of ordinary skill in the art to produce said antibody polypeptides.

The host cell may be co-transfected with the two expression vectors described above, the first expression vector containing DNA encoding a promoter (optionally as a component of a eukaryotic or prokaryotic operon) and a light chain-derived polypeptide and the second vector containing DNA encoding a promoter (optionally as a component of a eukaryotic or prokaryotic operon) and a heavy chain-derived polypeptide. The two vectors contain different selectable markers, but preferably achieve substantially equal expression of the heavy and light chain polypeptides. Alternatively, a single vector may be used, the vector including DNA encoding both the heavy and light chain polypeptides. The coding sequences for the heavy and light chains may comprise cDNA, genomic DNA, or both.

The host cells used to express the antibody polypeptides may be either a bacterial cell such as E. coli, or a eukaryotic cell such as P. pastoris. In one embodiment, a mammalian cell of a well-defined type for this purpose, such as a myeloma cell, a CHO cell line, a NS0 cell line, or a HEK293 cell line may be used.

The general methods by which the vectors may be constructed, transfection methods required to produce the host cell and culturing methods required to produce the antibody polypeptides from said host cells all include conventional techniques. Although preferably the cell line used to produce the antibody is a mammalian cell line, any other suitable cell line, such as a bacterial cell line such as an E. coli-derived bacterial strain, or a yeast cell line, may alternatively be used.

Similarly, once produced the antibody polypeptides may be purified according to standard procedures in the art, such as for example cross-flow filtration, ammonium sulphate precipitation, affinity column chromatography, hydrophobic interaction chromatography (“HIC”), and the like.

The antibody polypeptides described herein may also be used for the design and synthesis of either peptide or non-peptide mimetics that would be useful for the same therapeutic applications as the antibody polypeptides of the disclosure (See, for example, Saragobi et al., Science, 253:792-795 (1991), the contents of which are herein incorporated by reference in its entirety).

In another embodiment, the present disclosure contemplates methods for humanizing antibody heavy and light chains which bind to CoV-S. Exemplary methods for humanizing antibody heavy and light chains that may be applied to anti-CoV-S antibodies are identified herein and are conventional in the art.

### H. Screening Assays

The screening assays described here may be used to identify high affinity anti-CoV-S Abs which may be useful in the treatment of diseases and disorders associated with CoV-S in subjects exhibiting symptoms of a CoV-S associated disease or disorder.

In some embodiments, the antibody is used as a diagnostic tool. The antibody can be used to assay the amount of CoV-S present in a sample and/or subject. As will be appreciated by one of skill in the art, such antibodies need not be neutralizing antibodies. In some embodiments, the diagnostic antibody is not a neutralizing antibody. In some embodiments, the diagnostic antibody binds to a different epitope than the neutralizing antibody binds to. In some embodiments, the two antibodies do not compete with one another.

In some embodiments, the antibodies disclosed herein are used or provided in an assay kit and/or method for the detection of CoV-S in mammalian tissues or cells in order to screen/diagnose for a disease or disorder associated with changes in levels of CoV-S. The kit comprises an antibody that binds CoV-S and means for indicating the binding of the antibody with CoV-S, if present, and optionally CoV-S protein levels. Various means for indicating the presence of an antibody can be used. For example, fluorophores, other molecular probes, or enzymes can be linked to the antibody and the presence of the antibody can be observed in a variety of ways. The method for screening for such disorders can involve the use of the kit, or simply the use of one of the disclosed antibodies and the determination of whether the antibody binds to CoV-S in a sample. As will be appreciated by one of skill in the art, high or elevated levels of CoV-S will result in larger amounts of the antibody binding to CoV-S in the sample. Thus, degree of antibody binding can be used to determine how much CoV-S is in a sample. Subjects or samples with an amount of CoV-S that is greater than a predetermined amount (e.g., an amount or range that a person without a CoV-S-related disorder would have) can be characterized as having a CoV-S-mediated disorder.

The present disclosure further provides for a kit for detecting binding of an anti-CoV-S antibody of the disclosure to CoV-S. In particular, the kit may be used to detect the presence of CoV-S specifically reactive with an anti-CoV-S antibody of the disclosure or an immunoreactive fragment thereof. The kit may also include an antibody bound to a substrate, a secondary antibody reactive with the antigen and a reagent for detecting a reaction of the secondary antibody with the antigen. Such a kit may be an ELISA kit and can comprise the substrate, primary and secondary antibodies when appropriate, and any other necessary reagents such as detectable moieties, enzyme substrates, and color reagents, for example as described herein. The diagnostic kit may also be in the form of an immunoblot kit. The diagnostic kit may also be in the form of a chemiluminescent kit (Meso Scale Discovery, Gaithersburg, MD). The diagnostic kit may also be a lanthanide-based detection kit (PerkinElmer, San Jose, CA).

A skilled clinician would understand that a biological sample includes, but is not limited to, sera, plasma, urine, fecal sample, saliva, mucous, pleural fluid, synovial fluid, and spinal fluid.

### I. Methods of Ameliorating or Reducing Symptoms of, or Treating, or Preventing, Diseases and Disorders Associated with CoV-S

The present disclosure provides methods for ameliorating or reducing the symptoms of, or treating, or preventing, diseases and disorders associated with CoV-S. The methods comprise administering an antibody, or antigen-binding fragment thereof, that display broad activity against all SARS-CoV-2 variants of concern (including the Omicron/BA.1 variant).

In some embodiments, an antibody or antigen-binding fragment thereof is capable of binding to the spike protein of a corona virus (CoV-S). In some embodiments, the CoV-S is the spike protein of SARS-CoV (“SARS-CoV-S”) and/or the spike protein of SARS-CoV-2 (“SARS-CoV-2-S”)

In certain embodiments, an antibody or antigen-binding fragment thereof is capable of binding to a SARS-CoV-2 variant. In some embodiments, the SARS-CoV-2-S is a B.1.1.7 variant, a B.1.351 variant, a B.1.1.28 variant, a B.1.429 variant, a P.1 variant, a B.1.617 variant, a B.1.617.2 variant, a C.37 variant, a 1.621 variant, a AY.1 variant, a 1.623 variant, a C.36 variant, a A.27 variant, a AV.1 variant, a B.1.1.482 variant, a B.1.1.523 variant, a B.1.427 variant, a AY.4 variant, a AY.11 variant, a D614G variant, a B.1.1.529/BA.1 variant, a BA.4/5 variant, a BA.4.6 variant, a BA.7 variant, a BQ.1 variant, a BQ.1.1 variant, a BA.2.75 variant, a BN.1 variant, a XBB variant, a XBB.1 variant, a XBB.1.5 variant, a BJ.1 variant, a BM.1.1.1 variant, a BA.2.3.20 variant, a BF.7 variant, a XBC variant, a CH.1.1 variant, a XBB.1.16 variant, a XBB.1.5.10 variant, a XBB.1.5.1 variant, a XBB.2.3 variant, a FL.1.5.1 variant, a XBB.1.5.CONV0817 variant, a XBB.1.5.70 variant, a BA.2.86 variant, a BA2.86 CONV1207 variant, a HV.1 variant, a HK.3 variant, a JN.1 variant, a JN.4 variant, a JD.1.1 variant, a BA.2 variant, a JN.1.11.1 variant, a GE.1.2.1 variant, a JN.1.13.1 variant, a KQ.1 variant, a KP.1.1 variant, a KP.3 variant, a KP.2 variant, a JN.1.50 variant, a KP.3.1.1 variant, a XEC variant, a LB.1 variant, a MV.1 variant, a LP.8.1 variant, a MC.10.2. variant, a LF.7 variant, a LP.8.1.2 variant, a FL.15.1.1 variant, or a LF.7.2.1 variant.

Anti-CoV-S antibodies described herein, or antigen-binding fragments thereof, e.g., VYD2311, can also be administered in a therapeutically effective amount to patients in need of treatment of diseases and disorders associated with CoV-S in the form of a pharmaceutical composition as described in greater detail below.

The antibodies, or antigen-binding fragments thereof, can be useful in prophylactic or therapeutic applications as described herein, and may be administered by any route as described herein. Where the antibodies are used prophylactically, they may in certain embodiments be administered intramuscularly or subcutaneously. In certain embodiments, they are administered intramuscularly. Where the antibodies are used therapeutically, they may in certain embodiments be administered intravenously. Thus, in some embodiments, the methods for ameliorating or reducing the symptoms of, or treating diseases and disorders associated with CoV-S described herein comprise administering the antibody, or antigen-binding fragment thereof (e.g. VYD2311) intravenously. In some embodiments, the methods for preventing diseases and disorders associated with CoV-S described herein comprise administering the antibody, or antigen-binding fragment thereof (e.g. VYD2311) intramuscularly or subcutaneously. In certain embodiments, they are administered intramuscularly.

Symptoms of CoV infection may include fever, cough, runny nose, congestion, sore throat, bronchitis, pneumonia, shortness of breath, chest pain, headache, muscle ache, chills, fatigue, conjunctivitis, diarrhea, loss of smell, and loss of taste. Complications and/or diseases/disorders associated with coronavirus infection may include, for example, bronchitis, pneumonia, respiratory failure, acute respiratory failure, organ failure, multi-organ system failure, pediatric inflammatory multisystem syndrome, acute respiratory distress syndrome (a severe lung condition that causes low oxygen in the blood and organs), blood clots, cardiac conditions, myocardial injury, myocarditis, heart failure, cardiac arrest, acute myocardial infarction, dysrhythmias, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, gastrointestinal complications, aspergillosis, increased susceptibility to infection with another virus or bacteria, and/or pregnancy-related complications. Certain diseases and conditions, such as high blood pressure, type 1 diabetes, liver disease, overweight, chronic lung diseases including cystic fibrosis, pulmonary fibrosis, and asthma, compromised immune system due to transplant, use of an immunosuppressant, or HIV infection, and brain and nervous system condition, may increase the risk of CoV infection-associated complications and diseases. In some embodiments, the CoV infection is a chronic infection. In some embodiments, the CoV infection is an acute infection.

In some embodiments, the CoV infection is a long COVID. As used herein, the term “long COVID” refers to a chronic condition that occurs after SARS-CoV-2 infection and is present for at least 3 months. Long COVID can include a wide range of ongoing symptoms and conditions that can last weeks, months, or even years after infection.

Also, the subject anti-CoV-S antibodies and antigen-binding fragments may be used alone or in conjunction with other active agents, e.g., opioids and non-opioid analgesics such as NSAIDs to elicit analgesia. In some embodiments, aspirin and/or acetaminophen may be taken in conjunction with the subject anti-CoV-S antibody or antigen-binding fragment. Aspirin is another type of non-steroidal anti-inflammatory compound.

The subject antibodies potentially optionally may be combined with one or more of the following: (i) an antiviral drug, optionally, remdesivir, favipiravir, darunavir, nelfinavir, saquinavir, lopinavir, nirmatrelvir, or ritonavir; (ii) an antihelminth drug, optionally ivermectin; (iii) an antiparasitic drug, optionally hydroxychloroquine, chloroquine, or atovaquone; (iv) antibacterial vaccine, optionally the tuberculosis vaccine BCG; or (v) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (vi) an antihistamine drug, optionally bepotastine; (vii) an ACE inhibitor, which is optionally moexipril; or (viii) a drug that inhibits priming of CoV-S, optionally a serine protease inhibitor, further optionally nafamostat. in order to increase or enhance pain management. This may allow for such analgesic compounds to be administered for longer duration or at reduced dosages thereby potentially alleviating adverse side effects associated therewith.

In some embodiments, the ant-CoV-S antibody, or antigen-binding fragment thereof, is administered in combination with one or more existing and/or approved therapeutics for treating COVID. In one embodiment, the administration is at the same time. In one embodiment, the administration is sequential. In some embodiments, the antibody, or antigen-binding fragment thereof, is administered in combination with Paxlovid (nirmatrelvir/ritonavir), Remdesivir, Molnupiravir, or any combinations thereof. In some embodiments, the combination treatment regimens with existing and/or approved COVID therapeutics, e.g., Paxlovid, reduces the risk of COVID rebound, i.e, the recurrence of COVID symptoms.

In some embodiments, the anti-CoV-S antibodies and antigen-binding fragments disclosed herein are administered in combination with one or more antibodies as described in U.S. Provisional Application No. 63/143,456, filed on Jan. 29, 2021, and in U.S. Provisional Application No. 63/401,942, filed on Aug. 29, 2022, the entire contents of which have been incorporated herein by reference. In some embodiments, the antibody to be administered in combination with the anti-CoV-S antibodies and antigen-binding fragments disclosed herein is ADI-58125, as described in U.S. Provisional Application No. 63/143,456. In some embodiments, the antibody to be administered in combination with the anti-CoV-S antibodies and antigen-binding fragments disclosed herein is VYD222, as described in U.S. Provisional Application No. 63/401,942. In one embodiment, the antibodies are administered at the same time. In another embodiment, the antibodies are administered sequentially.

The subject to which the pharmaceutical formulation is administered can be, e.g., any human or non-human animal needing such treatment, prevention and/or amelioration, or who would otherwise benefit from the inhibition or attenuation of CoV-S-mediated activity. For example, the subject can be an individual that is diagnosed with, or who is deemed to be at risk of being afflicted by any of the aforementioned diseases or disorders. In some instances the subject may be in an advanced state of CoV infection, e.g., a subject who is on a ventilator. In some embodiments, the subject is an immunocompetent subject. In some embodiments, the subject is an immunocompromised subject. In some embodiments, the subject is at risk of exposure to SARS-CoV, SARS-CoV-2, and/or another coronavirus. In some instances, the subject can be one having one or more risk factors (such as advanced age, obesity, diabetes, etc, and others previously identified) which correlate to a poor CoV treatment or recovery prognosis and/or progression to severe infections. In some embodiments, the subject is not responsive or ineligible for treatment with existing and/or approved therapeutics for treating COVID. In some embodiments, the subject is an adult. In some embodiment is an adolescent, e.g., at least 12 years of age or older and weighting at least 40 kg. The present disclosure further includes the use of any of the pharmaceutical formulations disclosed herein in the manufacture of a medicament for the treatment, prevention and/or amelioration of any disease or disorder associated with CoV or CoV-S activity (including any of the above-mentioned exemplary diseases, disorders and conditions).

### J. Administration

In one embodiment, the anti-CoV-S antibodies described herein, or CoV-S binding fragments thereof, e.g., VYD2311, are administered to a subject at a concentration of between 0.1 mg/mL and about any one of 0.5, 1, 5, 10, 15 20, 25, 30, 35, 40, 45, 50, 55, 60, 65, 70, 75, 80, 85, 90, 95, 100, 110, 120, 130, 140, 150, 160, 170, 180, 190, or 200 mg/mL, +/−10% error.

In another embodiment, the anti-CoV-S antibodies and fragments thereof, described herein are administered to a subject at a dose of between about 0.01 and 100.0 or 200.0 mg/kg of body weight of the recipient subject. In certain embodiments, depending on the type and severity of the CoV-S-related disease, about 1 μg/kg to 50 mg/kg (e.g., 0.1-20 mg/kg) of antibody is an initial candidate dosage for administration to the patient, whether, for example, by one or more separate administrations, or by continuous infusion. In another embodiment, about 1 μg/kg to 15 mg/kg (e.g., 0.1 mg/kg-10 mg/kg) of antibody is an initial candidate dosage for administration to the patient. A typical daily dosage might range from about 1 μg/kg to 100 mg/kg or more, depending on several factors, e.g., the particular mammal being treated, the clinical condition of the individual patient, the cause of the disorder, the site of delivery of the agent, the method of administration, the scheduling of administration, and other factors known to medical practitioners. However, other dosage regimens may be useful.

For example, in addition to the relative dosages (mg/kg) discussed herein, the subject anti-CoV-S antibodies and antigen-binding fragments thereof can be administered to a subject at an absolute dose (mg). Accordingly, in one embodiment, the anti-CoV-S antibodies and antigen-binding fragments thereof described herein are administered to a subject at a dose of between about 1 microgram and about 2000 milligrams regardless of the route of administration.

In some embodiments, the antibody, or antigen-binding fragment thereof, is administered at a dose of about 100 mg to 9000 mg, about 100 mg to 8500 mg, about 100 to 8000 mg, about 100 mg to 7500 mg, about 100 to 7000 mg, about 100 mg to 6500 mg, about 100 to 6000 mg, about 100 mg to 5500 mg, about 100 mg to 5000 mg, about 100 mg to 4500 mg, about 100 mg to 4000 mg, about 100 mg to about 3500 mg, about 100 mg to about 3000 mg, about 100 mg to about 2500 mg, about 100 mg to about 2000 mg, about 200 mg to about 1500 mg, about 300 mg to about 600 mg, about 500 mg to about 1200 mg, about 300 mg to about 1200 mg, about 500 to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg, about 4500 mg to about 5000 mg, about 5000 mg to about 5500 mg, about 5500 mg to about 6000 mg, about 6500 mg to about 7000 mg, about 7500 mg to about 8000 mg, about 8000 mg to about 8500 mg, or about 8500 mg to about 9000 mg. In some embodiments, the antibody, or antigen-binding fragment thereof, is administered at a dose of about 3500 mg to about 9000 mg, about 4000 mg to about 9000 mg, about 4500 mg to about 9000 mg, about 5000 mg to about 9000 mg, about 5500 mg to about 9000 mg, about 6000 mg to about 9000 mg, about 6500 mg to about 9000 mg, about 7000 mg to about 9000 mg, about 8000 mg to about 9000 mg, about 8500 mg to about 9000 mg, about 5000 mg to about 8500 mg, about 5000 mg to about 8000 mg, about 5000 mg to about 7500 mg, about 5000 mg to about 7000 mg, about 5000 mg to about 6500 mg, about 5000 mg to about 6000 mg, about 5000 mg to about 5500 mg, about 6000 mg to about 9000 mg, about 6000 mg to about 8500 mg, about 6000 mg to about 8000 mg, about 6000 mg to about 7500 mg, about 6000 mg to about 7000 mg, about 6000 mg to about 6500 mg, about 6500 mg to about 9000 mg, about 6500 mg to about 8500 mg, about 6500 mg to about 8000 mg, about 6500 mg to about 7500 mg, about 6500 mg to about 7000 mg, about 7000 mg to about 9000 mg, about 7000 mg to about 8500 mg, about 7000 mg to about 8000 mg, about 7000 mg to about 7500 mg, about 7500 mg to about 9000 mg, about 7500 mg to about 8500 mg, about 7500 mg to about 8000 mg, about 8000 mg to about 9000 mg, or about 8000 mg to about 8500 mg.

In some embodiments, the antibody, or antigen-binding fragment thereof, is administered at a dose of about 100 mg, about 200 mg, about 300 mg, about 400 mg, about 450 mg, about 500 mg, about 600 mg, about 700 mg, about 800 mg, about 900 mg, about 1000 mg about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, about 6000 mg, about 6100 mg, about 6200 mg, about 6300 mg, about 6400 mg, about 6500 mg, about 6600 mg, about 6700 mg, about 6800 mg, about 6900 mg, about 7000 mg, about 7100 mg, about 7200 mg, about 7300 mg, about 7400 mg, about 7500 mg, about 7600 mg, about 7700 mg, about 7800 mg, about 7900 mg, about 8000 mg, about 8100 mg, about 8200 mg, about 8300 mg, about 8400 mg, about 8500 mg, about 8600 mg, about 8700 mg, about 8800 mg, about 8900 mg, or about 9000 mg.

In some embodiments, the antibody, or antigen-binding fragment thereof, is administered at a dose of about 250 mg to about 4500 mg.

In some embodiments, the antibody, or antigen-binding fragment thereof, is administered at a dose of about 250 mg to about 1000 mg intramuscularly. In some embodiments, the antibody, or antigen-binding fragment thereof, is administered at a dose of about 250 mg to about 1250 mg subcutaneously. In some embodiments, the antibody, or antigen-binding fragment thereof, is administered at a dose of about 250 mg to about 750 mg subcutaneously. In some embodiments, the antibody, or antigen-binding fragment thereof, is administered at a dose of about 250 mg to about 4500 mg intravenously. In some embodiments, the antibody, or antigen-binding fragment thereof, is administered at a dose of about 1500 mg to about 4500 mg intravenously. In some embodiments, the antibody, or antigen-binding fragment thereof, is administered at a dose of about 1500 mg to about 2000 mg intravenously. In some embodiments, the antibody, or antigen-binding fragment thereof, is administered at a dose of about 2000 mg to about 4500 mg intravenously.

In some embodiments, the antibody, or antigen-binding fragment thereof, is administered at a dose of about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg intramuscularly.

In some embodiments, the antibody, or antigen-binding fragment thereof, is administered at a dose of about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, or about 1250 mg subcutaneously.

In some embodiments, the antibody, or antigen-binding fragment thereof, is administered at a dose of about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, or about 4500 mg intravenously.

In some embodiments, the antibody, or antigen-binding fragment thereof, is administered intramuscularly. In some embodiments, the antibody, or antigen-binding fragment thereof, is administered subcutaneously. In some embodiments, the antibody, or antigen-binding fragment thereof, is administered intravenously. In one embodiment, the antibody, or antigen-binding fragment thereof, is administered via an IV push. In another embodiment, the antibody, or antigen-binding fragment thereof, is administered via an IV bolus. In some embodiments, the antibody, or antigen-binding fragment thereof, is administered via self-administration. In some embodiments, the antibody, or antigen-binding fragment thereof, is administered by a healthcare provider. In some embodiments, the antibody, or antigen-binding fragment thereof, is administered subcutaneously via self-administration.

In one embodiment, the antibody, or antigen-binding fragment thereof, is administered once. In one embodiment, the antibody, or antigen-binding fragment thereof, is administered weekly. In another embodiment, the antibody, or antigen-binding fragment thereof, is administered daily, weekly, every two weeks, monthly, every two months, or every three months. In another embodiment, the antibody, or antigen-binding fragment thereof, is administered every three months, every six months, or every year. In one embodiment, the antibody, or antigen-binding fragment thereof, is administered once every three months. In another embodiment, the antibody, or antigen-binding fragment thereof, is administered once every six months. In yet another embodiment, the antibody, or antigen-binding fragment thereof, is administered once every year.

In some embodiments, the antibody, or antigen-binding fragment thereof, is administered intramuscularly every three months. In other embodiments, the antibody, or antigen-binding fragment thereof, is administered subcutaneously every three months.

In some embodiments, the antibody, or antigen-binding fragment thereof, is administered at a loading dose followed by at least one maintenance dose. In some embodiments, the maintenance dose is administered every month, every two months, every three months, every six months, or every year after the loading dose. In some embodiments, the maintenance dose is administered every three months after the loading dose. In some embodiments, the maintenance dose is administered every six months after the loading dose.

In some embodiments, the antibody, or antigen-binding fragment thereof, is administered at a loading dose of about 100 mg to about 9000 mg, e.g., about 100 mg to about 9000 mg, about 100 mg to about 8500 mg, about 100 to about 8000 mg, about 100 mg to about 7500 mg, about 100 to about 7000 mg, about 100 mg to 6500 mg, about 100 to about 6000 mg, about 100 mg to about 5500 mg, about 100 mg to about 5000 mg, about 100 mg to about 4500 mg, about 100 mg to about 4000 mg, about 100 mg to about 3500 mg, about 100 mg to about 3000 mg, about 100 mg to about 2500 mg, about 100 mg to about 2000 mg, about 200 mg to about 1500 mg, about 300 mg to about 600 mg, about 500 mg to about 1200 mg, about 300 mg to about 1200 mg, about 500 to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg, about 4500 mg to about 5000 mg, about 5000 mg to about 5500 mg, about 5500 mg to about 6000 mg, about 6500 mg to about 7000 mg, about 7500 mg to about 8000 mg, about 8000 mg to about 8500 mg, or about 8500 mg to about 9000 mg, followed by at least one maintenance dose of about 100 mg to about 9000 mg, e.g., about 100 mg to about 9000 mg, about 100 mg to about 8500 mg, about 100 to about 8000 mg, about 100 mg to about 7500 mg, about 100 to about 7000 mg, about 100 mg to about 6500 mg, about 100 to about 6000 mg, about 100 mg to about 5500 mg, about 100 mg to about 5000 mg, about 100 mg to about 4500 mg, about 100 mg to about 4000 mg, about 100 mg to about 3500 mg, about 100 mg to about 3000 mg, about 100 mg to about 2500 mg, about 100 mg to about 2000 mg, about 200 mg to about 1500 mg, about 300 mg to about 600 mg, about 500 mg to about 1200 mg, about 300 mg to about 1200 mg, about 500 to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg, about 4500 mg to about 5000 mg, about 5000 mg to about 5500 mg, about 5500 mg to about 6000 mg, about 6500 mg to about 7000 mg, about 7500 mg to about 8000 mg, about 8000 mg to about 8500 mg, or about 8500 mg to about 9000 mg.

In some embodiments, the antibody, or antigen-binding fragment thereof, is administered at a loading dose of about 100 mg to about 4500 mg, e.g., about 100 mg to about 4500 mg, about 100 mg to about 4000 mg, about 100 mg to about 3500 mg, about 100 mg to about 3000 mg, about 100 mg to about 2500 mg, about 100 mg to about 2000 mg, about 100 to about 1500 mg, about 100 mg to about 1000 mg, about 1500 mg to about 4500 mg, about 250 mg to about 1000 mg, about 1000 mg to about 4500 mg, about 250 mg to about 750 mg, about 750 mg to about 4500 mg, about 200 mg to about 1500 mg, about 300 mg to about 600 mg, about 500 mg to about 1200 mg, about 300 mg to about 1200 mg, about 500 to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg, followed by at least one maintenance dose of about 100 mg to about 4500 mg, e.g., about 100 mg to about 4500 mg, about 100 mg to about 4000 mg, about 100 mg to about 3500 mg, about 100 mg to about 3000 mg, about 100 mg to about 2500 mg, about 100 mg to about 2000 mg, about 100 to about 1500 mg, about 100 mg to about 1000 mg, about 1500 mg to about 4500 mg, about 250 mg to about 1000 mg, about 1000 mg to about 4500 mg, about 250 mg to about 750 mg, about 750 mg to about 4500 mg, about 200 mg to about 1500 mg, about 300 mg to about 600 mg, about 500 mg to about 1200 mg, about 300 mg to about 1200 mg, about 500 to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg.

In some embodiments, the antibody, or antigen-binding fragment thereof, is administered intravenously at a loading dose of about 1500 mg to about 4500 mg, e.g., about 1500 mg to about 2000 mg, about 1500 mg to about 2500 mg, about 1500 to about 3000 mg, about 1500 mg to about 3500 mg, about 1500 to about 4000 mg, about 2000 mg to about 4500 mg, about 2000 mg to about 4000 mg, about 2000 mg to about 3500 mg, about 2000 mg to about 3000 mg, about 2500 mg to about 4500 mg, about 2500 mg to about 4000 mg, about 2500 mg to about 3500 mg, about 3000 mg to about 4500 mg, about 3000 mg to about 4000 mg, about 3500 mg to about 4500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg, followed by at least one maintenance dose of about 1500 mg to about 4500 mg, e.g., about 1500 mg to about 2000 mg, about 1500 mg to about 2500 mg, about 1500 to about 3000 mg, about 1500 mg to about 3500 mg, about 1500 to about 4000 mg, about 2000 mg to about 4500 mg, about 2000 mg to about 4000 mg, about 2000 mg to about 3500 mg, about 2000 mg to about 3000 mg, about 2500 mg to about 4500 mg, about 2500 mg to about 4000 mg, about 2500 mg to about 3500 mg, about 3000 mg to about 4500 mg, about 3000 mg to about 4000 mg, about 3500 mg to about 4500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg.

In some embodiments, the antibody, or antigen-binding fragment thereof, is administered intramuscularly at a loading dose of about 250 mg to about 1000 mg, e.g., about 300 mg to 1000 mg, about 350 mg to 1000 mg, about 400 mg to about 1000 mg, about 450 mg to about 1000 mg, about 500 mg to about 1000 mg, about 550 mg to about 1000 mg, about 600 mg to about 1000 mg, about 700 mg to about 1000 mg, about 800 mg to about 1000 mg, about 900 mg to about 1000 mg, about 250 mg to about 900 mg, about 250 mg to about 800 mg, about 250 mg to about 700 mg, about 250 mg to about 600 mg, about 250 mg to about 500 mg, followed by at least one maintenance dose of about 250 mg to about 1000 mg, e.g., about 300 mg to 1000 mg, about 350 mg to 1000 mg, about 400 mg to about 1000 mg, about 450 mg to about 1000 mg, about 500 mg to about 1000 mg, about 550 mg to about 1000 mg, about 600 mg to about 1000 mg, about 700 mg to about 1000 mg, about 800 mg to about 1000 mg, about 900 mg to about 1000 mg, about 250 mg to about 900 mg, about 250 mg to about 800 mg, about 250 mg to about 700 mg, about 250 mg to about 600 mg, about 250 mg to about 500 mg.

In some embodiments, the antibody, or antigen-binding fragment thereof, is administered subcutaneously at a loading dose of about 250 mg to about 1250 mg, e.g., about 300 mg to 1250 mg, about 350 mg to 1250 mg, about 400 mg to about 1250 mg, about 450 mg to about 1250 mg, about 500 mg to about 1250 mg, about 550 mg to about 1250 mg, about 600 mg to about 1250 mg, about 700 mg to about 1250 mg, about 800 mg to about 1250 mg, about 900 mg to about 1250 mg, about 1000 mg to about 1250 mg, about 250 mg to about 1200 mg, about 250 mg to about 1100 mg, about 250 mg to about 1000 mg, about 250 mg to about 900 mg, about 250 mg to about 800 mg, about 250 mg to about 750 mg, about 250 mg to about 700 mg, about 250 mg to about 600 mg, about 250 mg to about 500 mg, followed by at least one maintenance dose of about 250 mg to about 1250 mg, e.g., about 300 mg to 1250 mg, about 350 mg to 1250 mg, about 400 mg to about 1250 mg, about 450 mg to about 1250 mg, about 500 mg to about 1250 mg, about 550 mg to about 1250 mg, about 600 mg to about 1250 mg, about 700 mg to about 1250 mg, about 800 mg to about 1250 mg, about 900 mg to about 1250 mg, about 1000 mg to about 1250 mg, about 250 mg to about 1200 mg, about 250 mg to about 1100 mg, about 250 mg to about 1000 mg, about 250 mg to about 900 mg, about 250 mg to about 800 mg, about 250 mg to about 750 mg, about 250 mg to about 700 mg, about 250 mg to about 600 mg, about 250 mg to about 500 mg. In some embodiments, the antibody, or antigen-binding fragment thereof, is administered intravenously at a loading dose of about 1500 mg to about 4500 mg, followed by at least one maintenance dose of about 1500 mg to about 4500 mg, every month, every two months, every three months, every six months, or every year after the loading dose.

In some embodiments, the antibody, or antigen-binding fragment thereof, is administered intramuscularly at a loading dose of about 250 mg to about 1000 mg, followed by at least one maintenance dose of about 250 mg to about 1000 mg, every month, every two months, every three months, every six months, or every year after the loading dose.

In some embodiments, the antibody, or antigen-binding fragment thereof, is administered subcutaneously at a loading dose of about 250 mg to about 1250 mg, followed by at least one maintenance dose of about 250 mg to about 1250 mg, every month, every two months, every three months, every six months, or every year after the loading dose.

In one embodiment, the antibody, or antigen-binding fragment thereof, is administered weekly for about four weeks, once weekly for about a month, weekly for about 5 weeks, weekly for about 6 weeks, weekly for about 7 weeks, or weekly for about two months.

In another embodiment, the anti-CoV-S antibodies described herein, or anti-CoV-S antigen-binding fragments thereof, are administered to a recipient subject with a frequency of once every twenty-six weeks or less, such as once every sixteen weeks or less, once every eight weeks or less, once every four weeks or less, once every two weeks or less, once every week or less, or once daily or less.

In some embodiments, administering the antibody, or antigen-binding fragment thereof, or composition comprising the same, results in about 30%, about 40%, about 50%, about 60%, or about 70% relative risk reduction, e.g., for at least 2 months, at least 4 months, at least 6 months, at least 8 months, at least 10 months, or at least 12 months.

According to preferred embodiments, the antibody containing medicament or pharmaceutical composition is peripherally administered to a subject via a route selected from one or more of: orally, sublingually, buccally, topically, rectally, via inhalation, transdermally, subcutaneously, intravenously, intra-arterially, or intramuscularly, via intracardiac administration, intraosseously, intradermally, intraperitoneally, transmucosally, vaginally, intravitreally, epicutaneously, intra-articularly, peri-articularly, or locally.

Fab fragments may be administered every two weeks or less, every week or less, once daily or less, multiple times per day, and/or every few hours. In one embodiment, a patient receives Fab fragments of 0.1 mg/kg to 40 mg/kg per day given in divided doses of 1 to 6 times a day, or in a continuous perfusion form, effective to obtain desired results.

It is to be understood that the concentration of the antibody or Fab administered to a given patient may be greater or lower than the exemplary administration concentrations set forth above.

A person of skill in the art would be able to determine an effective dosage and frequency of administration through routine experimentation, for example guided by the disclosure herein and the teachings in, Goodman & Gilman's The Pharmacological Basis of Therapeutics, Brunton, L. L. et al. editors, 11^{th} edition, New York, New York: McGraw-Hill (2006); Howland, R. D. et al., Pharmacology, Volume 864, Lippincott's illustrated reviews., Philadelphia, PA: Lippincott Williams & Wilkins (2006); and Golan, D. E., Principles of pharmacology: the pathophysiologic basis of drug therapy, Philadelphia, PA: Lippincott Williams & Wilkins (2007).

In another embodiment, the anti-CoV-S antibodies described herein, or CoV-S binding fragments thereof, are administered to a subject in a pharmaceutical formulation. In a preferred embodiment, the subject is a human.

A “pharmaceutical composition” or “medicament” refers to a chemical or biological composition suitable for administration to a subject, preferably a mammal, more preferably a human. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal. In addition, administration can occur by means of injection, powder, liquid, gel, drops, or other means of administration.

In one embodiment, the anti-CoV-S antibodies described herein, or CoV-S binding fragments thereof, may be optionally administered in combination with one or more active agents. Such active agents include (i) an antiviral drug, optionally, remdesivir, favipiravir, darunavir, nelfinavir, saquinavir, lopinavir, nirmatrelvir, or ritonavir; (ii) an antihelminth drug, optionally ivermectin; (iii) an antiparasitic drug, optionally hydroxychloroquine, chloroquine, or atovaquone; (iv) antibacterial vaccine, optionally the tuberculosis vaccine BCG; or (v) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (vi) an antihistamine drug, optionally bepotastine; (vii) an ACE inhibitor, optionally moexipril; or (viii) a drug that inhibits priming of CoV-S, optionally a serine protease inhibitor, further optionally nafamostat.

An anti-histamine can be any compound that opposes the action of histamine or its release from cells (e.g., mast cells). Anti-histamines include but are not limited to acrivastine, astemizole, azatadine, azelastine, betatastine, brompheniramine, buclizine, cetirizine, cetirizine analogues, chlorpheniramine, clemastine, CS 560, cyproheptadine, desloratadine, dexchlorpheniramine, ebastine, epinastine, fexofenadine, HSR 609, hydroxyzine, levocabastine, loratadine, methscopolamine, mizolastine, norastemizole, phenindamine, promethazine, pyrilamine, terfenadine, and tranilast.

In CoV infection, respiratory symptoms are often exacerbated by additional bacterial infection. Therefore, such active agents may also be antibiotics, which include but are not limited to amikacin, aminoglycosides, amoxicillin, ampicillin, ansamycins, arsphenamine, azithromycin, azlocillin, aztreonam, bacitracin, carbacephem, carbapenems, carbenicillin, cefaclor, cefadroxil, cefalexin, cefalothin, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalosporins, chloramphenicol, cilastatin, ciprofloxacin, clarithromycin, clindamycin, cloxacillin, colistin, co-trimoxazole, dalfopristin, demeclocycline, dicloxacillin, dirithromycin, doripenem, doxycycline, enoxacin, ertapenem, erythromycin, ethambutol, flucloxacillin, fosfomycin, furazolidone, fusidic acid, gatifloxacin, geldanamycin, gentamicin, glycopeptides, herbimycin, imipenem, isoniazid, kanamycin, levofloxacin, lincomycin, linezolid, lomefloxacin, loracarbef, macrolides, mafenide, meropenem, methicillin, metronidazole, mezlocillin, minocycline, monobactams, moxifloxacin, mupirocin, nafcillin, neomycin, netilmicin, nitrofurantoin, norfloxacin, ofloxacin, oxacillin, oxytetracycline, paromomycin, penicillin, penicillins, piperacillin, platensimycin, polymyxin B, polypeptides, prontosil, pyrazinamide, quinolones, quinupristin, rifampicin, rifampin, roxithromycin, spectinomycin, streptomycin, sulfacetamide, sulfamethizole, sulfanilamide, sulfasalazine, sulfisoxazole, sulfonamides, teicoplanin, telithromycin, tetracycline, tetracyclines, ticarcillin, tinidazole, tobramycin, trimethoprim, trimethoprim-sulfamethoxazole, troleandomycin, trovafloxacin, and vancomycin.

Active agents also include aldosterone, beclomethasone, betamethasone, corticosteroids, cortisol, cortisone acetate, deoxycorticosterone acetate, dexamethasone, fludrocortisone acetate, glucocorticoids, hydrocortisone, methylprednisolone, prednisolone, prednisone, steroids, and triamcinolone. Any suitable combination of these active agents is also contemplated.

A “pharmaceutical excipient” or a “pharmaceutically acceptable excipient” is a carrier, usually a liquid, in which an active therapeutic agent is formulated. In one embodiment, the active therapeutic agent is a humanized antibody described herein, or one or more fragments thereof. The excipient generally does not provide any pharmacological activity to the formulation, though it may provide chemical and/or biological stability, and release characteristics. Exemplary formulations can be found, for example, in Remington's Pharmaceutical Sciences, Gennaro, A. editor, 19^{th} edition, Philadelphia, PA: Williams and Wilkins (1995), which is incorporated by reference.

As used herein “pharmaceutically acceptable carrier” or “excipient” includes any and all solvents, dispersion media, coatings, antibacterial and antifungal agents, isotonic, and absorption delaying agents that are physiologically compatible. In one embodiment, the carrier is suitable for parenteral administration. Alternatively, the carrier can be suitable for intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the disclosure is contemplated. Supplementary active compounds can also be incorporated into the compositions.

Pharmaceutical compositions typically must be sterile and stable under the conditions of manufacture and storage. The disclosure contemplates that the pharmaceutical composition is present in lyophilized form. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The disclosure further contemplates the inclusion of a stabilizer in the pharmaceutical composition. The proper fluidity can be maintained, for example, by the maintenance of the required particle size in the case of dispersion and by the use of surfactants.

In many cases, it will be preferable to include isotonic agents, for example, sugars, polyalcohols such as mannitol and sorbitol, or sodium chloride in the composition. Absorption of the injectable compositions can be prolonged by including an agent that delays absorption, for example, monostearate salts and gelatin. Moreover, the alkaline polypeptide can be formulated in a time-release formulation, for example in a composition that includes a slow-release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, polylactic and polyglycolic copolymers (“PLG”). Many methods for the preparation of such formulations are known to those skilled in the art.

For each of the recited embodiments, the compounds can be administered by a variety of dosage forms. Any biologically acceptable dosage form known to persons of ordinary skill in the art, and combinations thereof, are contemplated. Examples of such dosage forms include, without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

### K. Kits

In certain aspects, the instant disclosure provides kits comprising an antibody, or antigen-binding fragment thereof, of the present disclosure, as described herein, or an isolated nucleic acid molecule, e.g., an isolated mRNA molecule, encoding the antibody or antigen-binding fragment thereof, and a package insert with instructions to perform any of the methods described herein.

In some embodiments, the kits include instructions for using the kit. The instructions will generally include information about the use of the kit for treating and/preventing infection by SARS-CoV, SARS-CoV-2, and/or another coronavirus. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters.

In some embodiments, the kit includes a pharmaceutical formulation including an antibody, or antigen-binding fragment thereof, or an isolated nucleic acid molecule, e.g., an isolated mRNA molecule, encoding the antibody or antigen-binding fragment thereof, an additional therapeutic agent, and a package insert with instructions to perform any of the methods described herein.

In some embodiments, the kit can comprise formulation components for parenteral, subcutaneous, intramuscular or intravenous administration, e.g., sealed in a vial in a form ready for loading into a syringe and administration to a subject. In some embodiments, the kits can contain one or more, e.g., two, three, four, or five or more, vials, wherein each vial contains a single unit dose for administration to a subject. In some embodiments, the kit can contain one or more, e.g., two, three, four, or five or more, unit dose forms or vials as described herein.

The vial can be of any size. In some embodiments, the vial is about 1 mL, about 2 mL, about 4 mL, about 8 mL, about 12 mL, about 16 mL, about 20 mL, or about 24 mL in volume.

In some embodiments, each vial comprises about 100 mg, about 200 mg, about 300 mg, about 400 mg, about 450 mg, about 500 mg, about 600 mg, about 700 mg, about 800 mg, about 900 mg, about 1000 mg, about 1500 mg, about 2000 mg, about 2500 mg, about 2600 mg, about 2700 mg, about 3000 mg, about 3500 mg, about 4000 mg, about 4500 mg, about 5000 mg, about 5500 mg, about 6000 mg, about 6500 mg, about 7000 mg, about 7500 mg, about 8000 mg, about 8500 mg, or about 9000 mg of the antibody or antigen-binding fragment thereof.

The kit may be packaged in a number of different configurations such as one or more containers in a single box. The different components can be combined, e.g., according to instructions provided with the kit. The components can be combined according to a method described herein, e.g., to prepare and administer a pharmaceutical composition.

In some embodiments, the kit can comprise one or more containers with appropriate positive and negative controls or control samples, to be used as standard(s) for detection, calibration, or normalization.

The kit can further comprise a second container comprising a pharmaceutically-acceptable buffer, such as (sterile) phosphate-buffered saline, Ringer's solution, or dextrose solution; and other suitable additives such as penetration enhancers, carrier compounds and other pharmaceutically acceptable carriers or excipients, as described herein. It can further include other materials desirable from a commercial and user standpoint, including other buffers, diluents, filters, and package inserts with instructions for use. The kit can also include a drug delivery system such as liposomes, micelles, nanoparticles, and microspheres. The kit can further include a delivery device, such as needles, syringes, pumps, and package inserts with instructions for use.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

The invention may be practiced in ways other than those particularly described in the foregoing description and examples. Numerous modifications and variations of the invention are possible in light of the above teachings and, therefore, are within the scope of the appended claims.

Certain anti-CoV-S antibody polynucleotides and polypeptides are disclosed in the sequence listing accompanying this patent application filing, and the disclosure of said sequence listing is herein incorporated by reference in its entirety.

The entire disclosure of each document cited (including patents, patent applications, journal articles, abstracts, manuals, books, or other disclosures) in the Background, Detailed Description, and Examples is herein incorporated by reference in their entireties.

The following examples are put forth so as to provide those of ordinary skill in the art with a complete disclosure and description of how to make and use the subject invention and are not intended to limit the scope of what is regarded as the invention. Efforts have been made to ensure accuracy with respect to the numbers used (e.g. amounts, temperature, concentrations, etc.), but some experimental errors and deviations should be allowed for. Unless otherwise indicated, parts are parts by weight, molecular weight is average molecular weight, temperature is in degrees centigrade; and pressure is at or near atmospheric.

### EXAMPLES

### Example 1: Antibody with Improved Binding Affinity, Neutralization Efficacy, and Colloidal Stability

Antibodies with improved binding affinity to four primary Omicron variants (XBB.1.5 RBD, BQ.1.1 RBD, BA.2 S1 and BA.4/5 S1) and improved neutralizing potency against selected Omicron variants (XBB.1.5, XBB.1.5.10, CONV0817, HV.1, HK.3, BA.2.86, JN.1, JN.4 and BA. 2.86 CONV1207) were selected. Based on the in vitro neutralization breath and potency, VYD2311 was selected as a lead candidate.

VYD2311 is a human IgG1 mAb that binds to an epitope in the RBD of the spike glycoprotein of SARS-CoV-2 that is highly conserved across the sarbecovirus subgenus since the emergence of Omicron. VYD2311 is an offspring from the same lineage as pemivibart (VYD222) and adintrevimab (ADG20) and has been further optimized for improved binding affinity to variants of concern (VOC), including Omicron variants (XBB.1.5 and BQ.1.1).

Competitive binding experiments have demonstrated that VYD2311 competes with hACE2 for binding to SARS-CoV-2 spike glycoprotein, indicating that it is likely to neutralize by direct receptor blocking.

Binding affinities for VYD2311 were determined for XBB.1.5 and BA.2.86 RBD and Spike trimer using the Carterra LSA SPR instrument at WuXi biologics. Antigen reagents were purchased from Acro Bio (Catalogue numbers: SPD-C5242, SPN-C524i, SPD-C5247 & SPN-C524y). A control antibody was used as a comparator. An Anti-His antibody was used as a positive control. All experiments were performed in 1×HBSTE-BSA (0.5 mg/mL). The Antibodies were captured on the chips surface using a CMDP sensor with anti-Human FC capture surface (Southern Biotech goat anti Hu IgG FC Cat #2014 01). BA.2.86 and XBB.1.5 RBD were buffer exchanged (into 1×HBSTE using Zeba 0.5 mL spin columns) and titrated from 100 nM down to 0.1 nM (7 points, 1:3 serial dilution). Association time was five minutes, followed by a dissociation time of 15 minutes. All experiments were performed at 25° C. Data were analyzed using Kinetics 1.9 software.

As shown in Table 1 below, VYD2311 has improved binding against the Omicron variants, XBB.1.5 and BA.2.86, in the sub-nanomolar range, which is about 10-20 fold higher than the control antibody.

TABLE 1
Binding Affinities for VYD2311

| | RBD Omicron | RBD BA.2.86 |
|---|---|---|
| Ligand | Mean K_{D} (M) | Mean K_{D} (M) |
| Control Antibody | 9.50E−09 | 1.5E−08 |
| VYD2311 | 3.7E−10 | 1.1E−09 |

The neutralization activity of VYD2311 against SARS CoV-2 pseudovirus was evaluated in the PhenoSense SARS-CoV-2 neutralizing antibody assay, as described in Huang et al., 2021, Sci Rep. 11(1):23921. The control antibody was used as a comparator. As shown in Table 2, VYD2311 demonstrates broad and potent in vitro neutralizing activity against all pseudotyped SARS-CoV-2 variants evaluated, including Omicron sublineages X₁BB. 1.5, HV.1, BA.2.86, JN.1, and JD.1.1. In particular, VYD2311 was able to potently neutralize SARS-CoV-2 variants with an IC50<0.02 μg/mL.

TABLE 2
In vitro Neutralization Activity for VYD2311

| | Control Antibody | VYD2311 |
|---|---|---|
| Pango Lineage | IC₅₀ (μg/mL) | IC₅₀ (μg/mL) |
| BA.1 | 0.1214 | 0.0130 ± 0.0018 |
| XBB.1.5 | 0.1043 | 0.0037 ± 0.0007 |
| XBB.1.16 | 0.0776 ± 0.0181 | 0.0034 ± 0.0007 |
| XBB.1.5.10 | 0.1081 ± 0.0355 | 0.0041 ± 0.0017 |
| XBB.1.5.1 | 0.0800 ± 0.0089 | 0.0037 ± 0.0004 |
| XBB.2.3 | 0.0870 ± 0.0155 | 0.0042 ± 0.0005 |
| FL.1.5.1 | 0.1021 ± 0.0088 | 0.0031 ± 0.0007 |
| XBB.1.5.CONV0817 | 0.1572 ± 0.0866 | 0.0081 ± 0.0035 |
| BA.2.86 | 0.1677 ± 0.0044 | 0.0032 ± 0.0006 |
| HV.1 | 0.0412 ± 0.0092 | 0.0024 ± 0.0009 |
| HK.3 | 0.0723 ± 0.0077 | 0.0040 ± 0.0018 |
| JN.1 | 0.0746 ± 0.0058 | 0.0048 ± 0.0009 |
| JD.1.1 | 0.1019 ± 0.0049 | 0.0034 ± 0.0012 |
| BA.2 | 0.0446 ± 0.0065 | 0.0017 ± 0.0007 |
| GE.1.2.1 | 0.0721 ± 0.0148 | 0.0032 ± 0.0006 |
| JN.1.13.1 | 0.2203 ± 0.0325 | 0.0089 ± 0.0007 |
| KQ.1 | 0.2085 ± 0.0303 | 0.0044 ± 0.0010 |
| KP.1.1 (run 1) | 0.1771 ± 0.0338 | 0.0054 ± 0.0007 |
| KP.3 (run 1) | 0.4196 ± 0.0950 | 0.0235 ± 0.0015 |

A second neutralization assay was performed with respect to KP.3, as it was determined that the initial neutralization assay conditions were not within specification for that variant. The KP.1.1 variant, whose conditions had been within specification, was also tested again in this second experimental run. Table 3 provides the IC₅₀ values for KP.3 from the second experimental run and the average IC₅₀ value (avg) for KP.1.1 from both experimental runs. Other virus lineages including KP.2, JN.1.50, KP.3.1.1 and LB.1 were also evaluated in additional neutralization assays. As shown in Table 3, VYD2311 demonstrates potent in vitro neutralizing activity against all pseudotyped SARS-CoV-2 variants evaluated.

TABLE 3
In vitro Neutralization Activity for VYD2311

| | Control Antibody | VYD2311 |
|---|---|---|
| Pango Lineage | IC₅₀ (μg/mL) | IC₅₀ (μg/mL) |
| KP.1.1 (avg) | 0.1742 ± 0.0287 | 0.0048 ± 0.0010 |
| KP.3 (run 2) | 0.2230 ± 0.0626 | 0.0117 ± 0.0016 |
| KP.2 | 0.1541 ± 0.0294 | 0.0089 ± 0.0013 |
| JN.1.50 | 0.0603 ± 0.0178 | 0.0034 ± 0.0001 |
| KP.3.1.1 | 0.2393 ± 0.1414 | 0.0420 ± 0.0117 |
| LB.1 | 0.1787 ± 0.1141 | 0.0166 ± 0.0044 |

The neutralization activity of VYD2311 against additional SARS CoV-2 variants was further evaluated. The control antibody was used as a comparator. As shown in Table 4, VYD2311 demonstrates broad and potent in vitro neutralizing activity against all pseudotyped SARS-CoV-2 variants evaluated, including Omicron sublineages JN.1, and XEC. As demonstrated above, VYD2311 was able to potently neutralize SARS-CoV-2 variants with an IC50<0.02 μg/mL.

TABLE 4
In vitro Neutralization Activity for VYD2311

| | Control Antibody | VYD2311 |
|---|---|---|
| Pango Lineage | IC₅₀ (μg/mL) | IC₅₀ (μg/mL) |
| SARS-CoV-2 D614G | 0.0077 ± 0.0013 | 0.0093 ± 0.0030 |
| B.1.617.2 (Delta) | 0.0039 ± 0.0007 | 0.0048 ± 0.0017 |
| JN.1 | 0.0714 ± 0.0160 | 0.0026 ± 0.0007 |
| XEC | 0.2347 ± 0.0178 | 0.0142 ± 0.0024 |

The neutralization activity of VYD2311 against additional SARS CoV-2 variants was evaluated. The control antibody was used as a comparator. As shown in Table 5, VYD2311 demonstrates broad and potent in vitro neutralizing activity against all pseudotyped SARS-CoV-2 variants evaluated, including Omicron sublineages JN.1, MV.1 and LF.7. As demonstrated above, VYD2311 was able to potently neutralize SARS-CoV-2 variants with an IC50<0.02 μg/mL.

TABLE 5
In vitro Neutralization Activity for VYD2311

| | Control Antibody | VYD2311 |
|---|---|---|
| Pango Lineage | IC₅₀ (μg/mL) | IC₅₀ (μg/mL) |
| SARS-CoV-2 D614G | 0.0119 ± 0.0035 | 0.0124 ± 0.0030 |
| B.1.617.2 (Delta) | 0.0064 ± 0.0017 | 0.0077 ± 0.0009 |
| JN.1 | 0.1043 ± 0.0319 | 0.0035 ± 0.0008 |
| MV.1 | 0.1453 ± 0.0389 | 0.0048 ± 0.0012 |
| LF.7 | 0.3146 ± 0.0493 | 0.0109 ± 0.0023 |

The neutralization activity of VYD2311 against additional SARS CoV-2 variants was evaluated. The control antibody was used as a comparator. As shown in Table 6, VYD2311 demonstrates broad and potent in vitro neutralizing activity against all pseudotyped SARS-CoV-2 variants evaluated, including Omicron sublineages JN.1, LP.8.1 and MC.10.2. VYD2311 was able to potently neutralize the currently dominant LP.8.1 variant of SARS-CoV-2 with an IC50<0.02 μg/mL. This is consistent with the potency observed for previous JN.1 sublineages and is within 2-fold of the control value for JN.1 in the same system. Consistent with all dominant variants for the past three years, LP.8.1 did not demonstrate any meaningful change to the neutralization activity of VYD2311 as the epitope they target remains structurally intact. VYD2311 was able to potently neutralize the MC.10.2 variant of SARS-CoV-2 with an IC50<0.064 μg/mL. MC.10.2 is currently the only variant with T376N.

TABLE 6
In vitro Neutralization Activity for VYD2311

| | Control Antibody | VYD2311 |
|---|---|---|
| Pango Lineage | IC₅₀ (μg/mL) | IC₅₀ (μg/mL) |
| SARS-CoV-2 D614G | 0.0090 ± 0.0010 | 0.0193 ± 0.0047 |
| B.1.617.2 (Delta) | 0.0072 ± 0.0014 | 0.0090 ± 0.0010 |
| JN.1 | 0.1016 ± 0.0199 | 0.0050 ± 0.0010 |
| LP.8.1 | 0.1901 ± 0.0438 | 0.0189 ± 0.0008 |
| MC.10.2 | 0.4479 ± 0.1179 | 0.0642 ± 0.0087 |

The neutralization activity of VYD2311 against additional SARS CoV-2 variants was evaluated. The control antibody was used as a comparator. As shown in Tables 7 and 8, VYD2311 demonstrates broad and potent in vitro neutralizing activity against all pseudotyped SARS-CoV-2 variants evaluated, including Omicron sublineages JN.1, LP.8.1.2 and LF.7.2.1. VYD2311 was able to potently neutralize the LF.7.2.1 variant with an IC50<0.0063 μg/mL and the LP.8.1.2 variant of SARS-CoV-2 with an IC50<0.0129 μg/mL.

TABLE 7
In vitro Neutralization Activity for VYD2311

| | Control Antibody | VYD2311 |
|---|---|---|
| Pango Lineage | IC₅₀ (μg/mL) | IC₅₀ (μg/mL) |
| SARS-CoV-2 D614G | 0.0071 ± 0.0013 | 0.0098 ± 0.0029 |
| B.1.617.2 (Delta) | 0.0051 ± 0.0003 | 0.0048 ± 0.0006 |
| JN.1 | 0.0836 ± 0.0162 | 0.0043 ± 0.0005 |
| LF.7.2.1 | 0.0795 ± 0.0184 | 0.0063 ± 0.0013 |

TABLE 8
In vitro Neutralization Activity for VYD2311

| | Control Antibody | VYD2311 |
|---|---|---|
| Pango Lineage | IC₅₀ (μg/mL) | IC₅₀ (μg/mL) |
| SARS-CoV-2 D614G | 0.0071 ± 0.0008 | 0.0163 ± 0.0019 |
| B.1.617.2 (Delta) | 0.0067 ± 0.0001 | 0.0083 ± 0.0014 |
| JN.1 | 0.0698 ± 0.0157 | 0.0053 ± 0.0015 |
| LP.8.1.2. | 0.1146 ± 0.0230 | 0.0129 ± 0.0023 |

The developability profile of VYD2311 was also analyzed, alongside the control antibody as a comparator antibody, using nanoDSF and isothermal DLS. These data were determined using the NanoTemper Prometheus Panta instrument. Samples were tested at 0.5 mg/mL in both 1×PBS pH 7.4 and 20 mM Histidine pH 5.6 buffers. Isothermal DLS was conducted at 25° C. with 10 acquisitions per capillary. The thermal melting experiments using nanoDSF were carried out at the same protein concentration and in the same buffers over a temperature range of 25-95° C., with a ramp rate: −1° C. per minute.

As shown in Table 9 (also see FIG. 1 and FIG. 2), VYD2311 showed a lower polydispersity (PDI) than the control antibody, as well as a higher T_{M} and Tₒₙ (the onset of melting) which leads to improved shelf life and half-life. These results suggest that VYD2311 surprisingly has more favorable thermal and colloidal stability properties in vitro and, thus, VYD2311 is more stable than previous antibodies.

TABLE 9
Mean datatsets for nanoDSF (Tₒₙ, T_{M}), Turbidity, DLS (Scattering & PDI) for n =
2, with standard deviation for VYD2311 & control antibody at 2 mg/mL PBS pH 7.4.

| | T_{M} (° C.) | | Tₒₙ | | T_{tb} | | Scattering | | PDI | |
|---|---|---|---|---|---|---|---|---|---|---|
| Test Antibody | Ø | σ | Ø | σ | Ø | σ | Ø | σ | Ø | σ |
| Control Ab | 59.987 | 0.060 | 54.371 | 0.268 | 59.845 | 0.000 | 55.211 | 0.002 | 0.110 | 0.030 |
| VYD2311 | 63.718 | 0.010 | 58.518 | 0.267 | 63.052 | 0.267 | 59.036 | 0.144 | 0.020 | 0.010 |

Additional analytical SEC and HIC results show that VYD2311 has a purity greater than 99% without aggregation, and an acceptable hydrophobic quality.

These data suggest that VYD2311 displays improved binding affinity and in vitro neutralization efficacy against all SARS-CoV-2 VOCs described to date, including the Omicron variants. In addition, VYD2311 has improved thermal and colloidal stability in vitro suggesting that there is less particle aggregation and phase separation for VYD2311 leading to the potential of smaller dosing volumes. In conclusion, this antibody, and antigen-binding fragments thereof, provides a promising candidate for therapeutic development and a framework for the development of vaccines that induce broadly neutralizing antibody responses.

### Example 2: Preclinical Safety Assessment

The safety of VYD2311 is being evaluated in 4 nonclinical studies: a GLP single dose rat toxicity/TK study with a 2-week recovery period; a GLP 22-day repeat dose rat toxicity/TK study with a 3-week recovery period; and 2 human (adult and fetal) TCR studies. The PK of VYD2311 is also being evaluated in an NHP study.

### Single-Dose Toxicity and Toxicokinetics Study in Sprague Dawley Rats

The TK of VYD2311 was measured in a GLP-compliant, single dose toxicity study in Sprague Dawley rats. The study included groups of 15 male and 15 female rats that were administered a single dose of placebo (formulation buffer) or VYD2311 by IV infusion at 450 mg/kg (10 mL/kg) or IM injection at 45 mg/kg (1 mL/kg). The control group received placebo by IV infusion, followed by IM injection. TK cohorts included 3/sex in the control group and 9/sex in the VYD2311-treated groups. On Study Day 2, 10 animals/sex were euthanized, and the remaining 5 animals/sex in each group were maintained an additional 14 days to assess reversibility of any drug-related effects.

Parameters evaluated included daily mortality and clinical observations, IM injection site observations via modified Draize scoring, body weights and food consumption, ophthalmoscopy, clinical pathology (at the time of necropsy), TK, gross pathology, organ weights, and histopathology. Blood samples were collected from TK animals at various timepoints (N=3 rats per sex per timepoint) for determination of serum drug concentrations and calculation of TK parameters. Blood samples for ADA analysis were also collected for potential future analysis to aid in data interpretation.

VYD2311 was well tolerated in a GLP-compliant, single-dose toxicity study in Sprague Dawley rats with a 2-week recovery period. No toxicologically meaningful findings were observed with IV administration of 450 mg/kg/dose or IM administration of 45 mg/kg/dose in male and female rats. Therefore, the NOAEL was reported as 450 mg/kg by IV infusion and 45 mg/kg by IM injection.

There were no VYD2311-related findings in clinical observations, body weight, food consumption, ophthalmology, hematology, coagulation, urinalysis, clinical chemistry, organ weights, gross pathology, and histopathology.

### Repeat-Dose Toxicity and Toxicokinetics Study in Sprague Dawley Rats

The TK of VYD2311 was measured in a GLP-compliant 22-day, repeat-dose toxicity study in Sprague Dawley rats. The study included groups of 15 male and 15 female rats that were administered placebo (formulation buffer) or VYD2311 by IV infusion at 450 mg/kg/dose (10 mL/kg) or IM injection at 45 mg/kg/dose (1 mL/kg) for 4 doses (Days 1, 8, 15, and 22). The control group received placebo by IV infusion, followed by IM injection. At the end of the treatment period, 10 animals/sex in each group were euthanized, and the remaining 5 animals/sex in each group were maintained an additional 21 days to assess reversibility of any drug-related effects.

Parameters evaluated included daily mortality and clinical observations, IM injection site observations via modified Draize scoring, body weights and food consumption, ophthalmoscopy, laboratory parameters, clinical pathology (at the time of necropsy), TK, gross pathology, organ weights, and histopathology. Blood samples were collected from TK animals at various timepoints (N=3 rats per sex per timepoint) for determination of serum drug concentrations and calculation of TK parameters. Blood samples for ADA analysis were also collected for potential future analysis to aid in data interpretation.

VYD2311 was well tolerated in a GLP-compliant, 22-day, repeat-dose toxicity study in Sprague Dawley rats with a 3-week recovery period. No toxicologically meaningful findings were observed with IV administration of 450 mg/kg/dose or IM administration of 45 mg/kg/dose in male and female rats. Therefore, the NOAEL was reported as 450 mg/kg by IV infusion and 45 mg/kg by IM injection.

There were no VYD2311-related findings in clinical observations, body weight, food consumption, ophthalmology, hematology, coagulation, urinalysis, clinical chemistry, organ weights, gross pathology, and histopathology.

Tissue Cross-Reactivity Study with Normal Adult Human Tissues

In the adult tissue TCR assay, biotinylated VYD2311 and biotinylated human IgG1 (control article) were tested at 2 concentrations (0.25 and 1.5 μg/mL) against a panel of 37 different frozen adult human tissues obtained from 3 different donors. These concentrations were chosen based on method development results which indicated that 0.25 μg/mL was the lowest concentration that produced the maximum and stable binding to the positive control tissue and 1.5 μg/mL was the highest concentration examined that did not yield nonspecific staining of the control samples and/or test tissues.

There was no cross-reactivity (off-target binding) of VYD2311 to normal adult human tissues in a TCR study. No specific binding of Biotin-VYD2311 and no specific Biotin VYD2311 or Biotin-Human IgG1 staining was observed in the adult human tissues tested. As the SARS-CoV-2 spike protein was not expected in normal human tissues, these findings were expected.

Tissue Cross-Reactivity Study with Normal Fetal Human Tissues

In the fetal tissue TCR assay, biotinylated VYD2311 and biotinylated motavizumab (control article) were tested at 2 concentrations (10 and 1 μg/mL) against a panel of 21 different frozen fetal human tissues obtained from 3 different donors. These concentrations were chosen based on method development results which indicated that 1 μg/mL was the lowest concentration that produced the maximum and stable binding to the positive control tissue and 10 μg/mL was the highest concentration examined that did not yield nonspecific staining of the control samples and/or test tissues.

There was no cross-reactivity (off-target binding) of VYD2311 to normal fetal human tissues in a TCR study. No specific binding of Biotin-VYD2311 and no Biotin VYD2311 or Biotin-motavizumab staining was observed in the fetal human tissues tested. As the SARS-CoV-2 spike protein was not expected in normal human tissues, these findings were expected.

### Example 3. Pharmacokinetic Analysis

### Pharmacokinetic Study in FcRn Tg32 SCID Mice

VYD2311 was evaluated in FcRn Tg32 SCID mice. Mice were allocated to different treatment groups (4 mice/group). One treatment group for each sex was administered a single dose of each antibody by IV injection at 60 mg/kg. Blood samples were collected at 5 minutes, 8 hours, 1, 3, 7, 14, 21, 28, 42, 56, 70, and 84 days post dose and processed to serum for PK analysis. All mice were euthanized after the last PK assessment on Day 84.

### Pharmacokinetic Study in Cynomolgus Monkeys

The PK and immunogenicity of VYD2311 were evaluated in cynomolgus monkeys (4 animals/group) following a single IV infusion (over 60 minutes) of 10 mg/kg to male cynomolgus monkeys. Serum samples were collected for PK and ADA analysis at various timepoints up to 98 days (≈5 half-lives) post dose. To support clinical dose selection and initiation of the FIH study, PK data from 1 half-life (21 days) were analyzed prior to completion of the study.

### Example 4. First In Human (FIH) Phase ½ Study

The Phase ½ study is a randomized, double-blind, placebo-controlled study to evaluate the safety, tolerability, pharmacokinetics, and immunogenicity of a SARS-CoV-2-directed monoclonal antibody, VYD2311, in healthy participants aged 18 to 65 years.

The study duration for each participant is approximately 7 months. Participants receive either a single dose of VYD2311 or placebo (normal saline), delivered by IV infusion or IM injection or SC injection. Participants stay overnight at the clinical research unit on the day prior to dosing until 24 hours after dosing (Day 2). Other in-person visits occur for Screening and on Days 7, 14, 21, 45, 65, and at 3 and 6 months after dosing. In addition, safety monitoring are performed by phone on Days 3 and 4 and monthly between Months 3 and 6.

A total of 10 participants are enrolled in up to 4 cohorts, such that up to 40 participants are randomized and receive investigational product. Participants in the study are randomized in an 8:2 fashion to receive a single IV or IM or SC dose of VYD2311 or placebo in 1 of 4 cohorts. The dose regimen of VYD2311 in each cohort is as follows:

| Treatment Arm | VYD2311 | Placebo |
|---|---|---|
| Treatment Name | VYD2311 | Normal saline |
| Type | Drug | Drug |
| Dose | Solution for injection | Solution for injection |
| Formulation/ | VYD2311 125 mg/mL in 10 mM | 0.9% sodium chloride |
| Unit Dose | L-histidine, 75 mM L-arginine HCl, | |
| Strength | 110 mM glycine, 10 mM L-methionine, | |
| | 0.03% polysorbate 80, pH 6.2 | |
| Dosage Level and | Cohort 1: 1000 mg IM | Matched volume and route |
| Route of | Cohort 2: 2000 mg IV | |
| Administration | Cohort 3: 4500 mg IV | |
| | Cohort 4: 1250 mg SC | |
| Use | Experimental | Placebo |

Cohort 1 is dosed first. The first 2 participants (sentinels) to be dosed in Cohort 1 of the study are randomized 1:1 to VYD2311 or placebo. The Investigator review available safety data (eg, AEs, vital signs, clinical laboratory, and physical examination findings) collected from the sentinel participants through at least 72 hours post dose for Cohort 1. If there are no safety concerns, the remaining participants in Cohort 1 as well as 2 sentinel participants in Cohort 2 may be dosed. The Investigator reviews Cohort 2 sentinel participant safety data through 24 hours post dose. If there are no safety concerns, the remaining participants in Cohort 2 may be dosed. Following completion of dosing in both Cohorts 1 and 2, the SRC reviews the available blinded safety and tolerability data through at least the Day 14 visit from all participants dosed in Cohort 1 and at least the Day 7 visit for all participants dosed in Cohort 2, including but not limited to, AEs, vital signs, and clinical safety laboratory tests, prior to making a recommendation on proceeding to dose escalation in Cohort 3. This range of routes of administrations and doses was tested to provide maximum flexibility in designing registrational pathways for both COVID-19 prophylaxis and treatment, while retaining a high barrier to resistance in the form of doses expected to accommodate significant evolutionary changes in SARS-CoV-2 viruses.

Participants were monitored closely for AEs, and blood samples are collected for PK and immunogenicity throughout the follow-up period.

Participants are eligible to be included in the study only if all of the following criteria apply:
Is a male or female participant aged 18 to 65 years, inclusive.
Has a body mass index 18.0 to 32.0 kg/m², inclusive.
Is in good health, with no clinically significant abnormalities as determined by the Investigator based on medical history, physical exam, vital signs, ECG, and laboratory values per study unit standard operating procedures.
Tests negative for current SARS-CoV-2 infection by rapid antigen test on screening and Day −1.
For participants assigned female sex at birth:
   Is not of childbearing potential, as defined in Section 10.4.1; OR
   Is of childbearing potential and practicing highly effective contraception for at least 28 days before dosing (Day 1) through 6 months after dosing and has negative results on pregnancy tests at Screening and on Day −1.
Is able and willing to provide written informed consent.
Is able to understand the study procedures and willing to adhere to all protocol requirements.

Participants are excluded from the study if any of the following criteria apply:
Has a known or suspected allergy, intolerance, or hypersensitivity to any component of the study drug, including excipients and closely related compounds (eg, other mAbs).
Intends to receive a COVID-19 vaccine/booster within 3 months of Day 1.
Is pregnant, breastfeeding, or seeking pregnancy while on study.
Has any chronic or significant medical condition that, in the assessment of the Investigator, might compromise participant safety or interfere with evaluation of the study drug or interpretation of participant safety/study results, including but not limited to significant neurologic, renal, hepatic, hematologic, immune, cardiac, pulmonary, metabolic, endocrine, psychiatric, vascular, or gastrointestinal disorders.
Has a history of a malignancy (or active malignancy), except for participants with basal cell carcinoma, squamous cell carcinoma, or carcinoma in situ of the cervix who have been treated and cured.
Has had any symptoms of acute respiratory illness (eg, cough, shortness of breath, sore throat, fatigue, loss of smell, fever), or other febrile illness within 2 weeks prior to dosing.
Has evidence of active infection with HIV, HBV, or HCV, as indicated by any of the following: positive antibody, antigen, or nucleic acid amplification test result for HIV; positive HBV surface antigen; positive HCV antibody with positive HCV RNA (positive antibody test with negative RNA test is not exclusionary).
Has current alcoholism or recreational drug use, including a positive test result for marijuana, amphetamines, barbiturates, cocaine, opiates, phencyclidine and benzodiazepines, or alcohol.
Is a current or former regular cigarette smoker (more than 5 cigarettes per day within the past 5 years). Individuals who currently or previously smoked 5 or fewer cigarettes per day are allowed if they agree to abstain from smoking during confinement at the CRU.
Has donated more than 500 mL of blood within 60 days before the scheduled dose of study drug.
Had major surgery within 30 days prior to study drug dosing or has planned surgeries within 12 months after planned study drug dosing.
Received any investigational drug or biologic within 30 days or 5 half-lives (whichever is longer) prior to Screening or planned administration of any investigational drug or biologic during the study period.
Received immunoglobulin or blood products within 6 months prior to Screening.
Previously received a mAb within 6 months or 5 half-lives (whichever is longer) prior to Screening or previously received pemivibart (VYD222) at any time.
Has taken prescription or OTC medications or supplements within 5 half-lives of the specific substance (or, if half-life is not known, within 48 hours) before the scheduled administration of study drug, with the following exceptions:
   Influenza or COVID-19 vaccination more than 14 days prior to dosing, or any other vaccine more than 4 weeks prior to dosing
   Hormonal contraceptives
   Standard of care use of acetaminophen/paracetamol
   Vitamins and other nutritional supplements that are not newly introduced (ie, have been taken for at least 30 days prior to enrollment)
   Other OTC medications that, in the judgement of the Investigator or medically qualified designee, will not impact the safety of the participant or data integrity of the study.
Screening or predose systolic blood pressure that repeatedly measures below approximately 100 mm Hg. If systolic blood pressure measures below 100 mm Hg, the measurement may be repeated twice. If the average of all three measurements is greater than or equal to 100 mm Hg, the participant may be considered eligible as determined by the Investigator or medically qualified designee.

The ongoing Phase ½ clinical trial, fully recruited (40 subjects) and with all planned doses administered, explores key clinical parameters for three different routes of administration (IV, IM, and SC) to support development for both prophylaxis and treatment of COVID-19 in broad patient populations. Participants received either a single dose of VYD2311 or placebo (normal saline), delivered by IV infusion, IM or SC injection. VYD2311 was administered at dose levels of 2000 mg IV, 4500 mg IV, 1000 mg IM and 1250 mg SC. Serum samples were collected for measurement of serum concentrations of active study drug and evaluation of PK.

Phase ½ clinical data to date is positive for both safety and pharmacokinetics, and is supported by antiviral activity data from standard virologic assessments. VYD2311 was well tolerated. All adverse events (AEs) identified in the pooled, blinded safety evaluation to date are mild or moderate; in severity with no serious or severe AEs reported. All AEs were deemed unrelated to study drug or, as expected and largely related to injection site erythema, injection site pain, injection site swelling, headache, dizziness, and infusion-related reactions, one of which (Grade 2) required an infusion interruption and was later restarted without any further reaction.

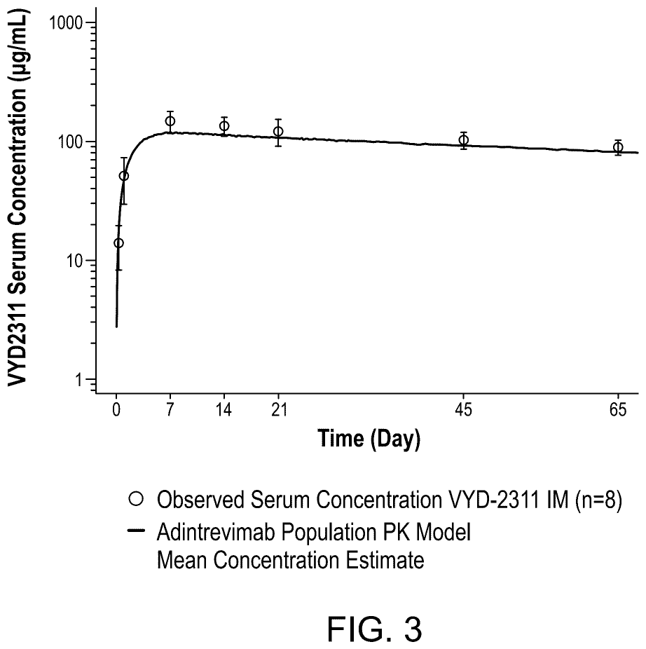

As of Day 65, serum concentrations remain high, indicating a potential long clinical dosing interval, and a potential substantial increase in observed half-life of VYD2311 relative to a control antibody. Analysis for the IM cohort (the most advanced cohort in time) is tracking generally with the PK profile of adintrevimab with an estimated in vivo half-life of 139 days (FIG. 3). PK analysis of VYD2311 intravenous and subcutaneous cohorts at earlier timepoints, at either similar doses subcutaneously or higher doses intravenously, are similarly and encouragingly tracking close to the estimated curves for adintrevimab thus far.

In vitro neutralization data generated continuously on VYD2311 demonstrate an average potency improvement for VYD2311 of approximately 17-fold compared to a control antibody. PK modelling was conducted of VYD2311 dosing regimens administered every 3 and 6 months across the following dose ranges: IV: 1500-4500 mg; IM: 250-1000 mg; SC: 250-750 mg. Effects of repeated dosing for IV, IM, and SC routes, as well as IV and IM loading doses were also modelled.

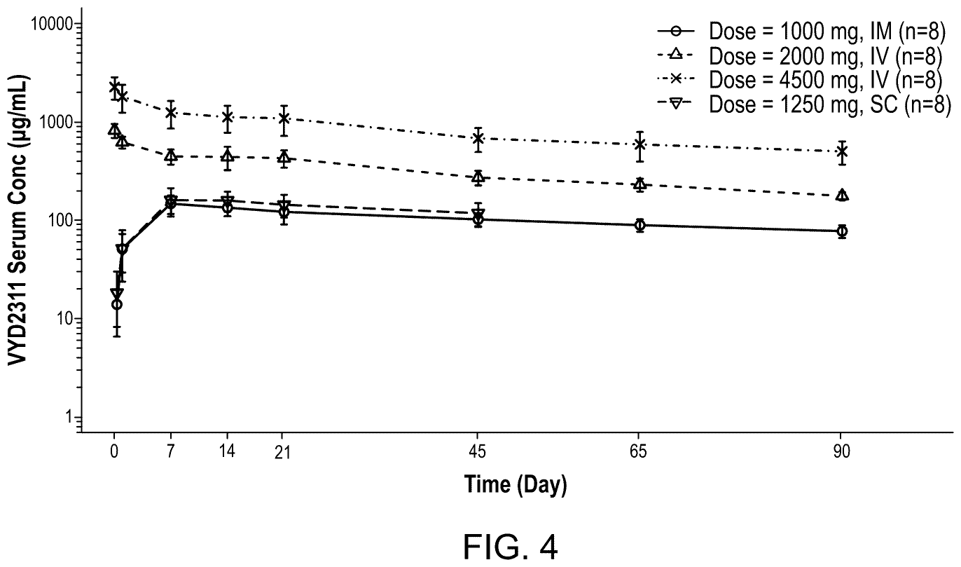

VYD2311 serum concentration data up to 90 days post dose in both IV cohorts and the IM cohort, as well as up to Day 45 days in the SC cohort, were available for PK analysis (FIG. 4). Post IV infusion, VYD2311 concentration achieved its peak level at the end of infusion then declined bi-exponentially. Following an IM or a SC injection, the median time to achieve peak concentration was approximately 7 days or 10 days, respectively. Due to the slow absorption, patterns of flip-flop PK were observed post an IM or a SC injection where VYD2311 concentration declines in a mono-exponential manner. A 2-compartment open model with first-order absorption rate (for IM and SC routes of administration) was able to fit the PK data from all 4 cohorts simultaneously. Based on the interim population PK modeling, VYD2311 has a clearance of 0.046 L/day and volume of distribution of 4.25 L, respectively. The absolute bioavailability of an IM or a SC injection was 78.0% and 62.4%, respectively. Based on preliminary post hoc parameters, the median terminal half-life of VYD2311 in the IV, IM and SC cohorts was 58 to 63 days, 73 days, and 68 days, respectively, which represents a substantial improvement over a control mAb (median estimate of terminal half-life in CANOPY was 43.6 days) with corresponding effects on the ability of the molecule to deliver substantial clinical antiviral activity to patients in need.

Table 10 below describes the clinical pharmacology differences between VYD2311 and the control antibody.

TABLE 10
Comparison of Key Pharmacokinetic Parameters and Potency against Dominant
Circulating Covid-19 Variants between VYD2311 and Control Antibody

| | Clearance | Volume of | Terminal half- | IC₅₀ against dominant |
|---|---|---|---|---|
| | (L/day) | distribution (L) | life (day) | circulating variants |
| VYD2311^{a} | 0.046 | 4.25 | 65.8 | KP.3.1.1: 42 ng/mL |
| | | | | LP.8.1: 18.9 ng/mL |
| | | | | XEC: 14.2 ng/mL |
| Control | 0.0888 | 5.27 | 43.6 | KP.3.1.1: 239.3 ng/mL |
| mAb ^{b} | | | | LP.8.1: 190.1 ng/mL |
| | | | | XEC: 234.7 ng/mL |

^{a}Clearance, volume of distribution, and terminal half-life were estimated and derived from the population PK model using interim Phase 1 data
^{b} Clearance, volume of distribution, and terminal half-life were estimated for the Phase 3 CANOPY Study participants by population PK analysis

As demonstrated in Table 10, there is an significant increase in half-life for VYD2311 as compared to the control mAb, allowing for a longer dosing interval, and a significant increase in potency against circulating variants, i.e., 5.7-16.5 times higher potency, for VYD2311 as compared to the control mAb, allowing for a potentially lower dose and a more patient-friendly dosing routes such as intramuscular or subcutaneous injection. In a clinical setting of treating an active infection, such long half-life can contribute to long-term viral suppression and aid in clearance and would represent a likely improvement of continuous viral suppression compared to approved or authorized alternatives following a single administration and with guaranteed compliance. In a prophylactic setting, such long in vivo half life would establish a mAb as a potentially safer, more effective, more durable alternative to active immunization via COVID-19 spike vaccination in those who do not mount an effective immune responses to vaccination and an equivalent or more effective alternative in those who do.

A formal estimate of in vivo half-life based on full Phase ½ clinical trial data confirmed the long half-life of VYD2311. At six months (end of study follow-up), serum concentrations of VYD2311 remained high and were observed to be substantially greater than that of the control antibody. Specifically, half-life estimates by cohort ranged from 61 days (high dose IV) to 76 days (IM) as compared to the estimated half-life of 49 days for the controlled antibody. In particular, the median half-life of VYD2311 at a dose of 1000 mg via IM administration is about 76 (with a range of about 68.5-90.7 days). The median half-life of VYD2311 at a dose of 1250 mg via SC administration is about 70.2 (with a range of about 55.4-83.7 days). The median half-life of VYD2311 at a dose of 2000 mg via IV administration is about 71.9 (with a range of about 55.4-77.8 days). The median half-life of VYD2311 at a dose of 4500 mg via IV administration is about 61.3 (with a range of about 41.8-69.1 days). VYD2311's long half-life could allow meaningful, long-term protection from symptomatic disease, potentially over multiple quarters, which is expected to be more durable than a COVID-19 vaccine, given rapid waning of protective benefit. High dose IV administration could similarly provide very long-term follow-up viral suppression in a COVID-19 treatment use case.

In addition to assessing safety and tolerability of VYD2311 across the various routes of administration, a comprehensive dose modeling analysis was conducted to support a rational dosing paradigm for VYD2311. A Cox model analysis was employed using data from the CANOPY Phase 3 clinical trial, incorporating both serum virus neutralizing antibody (sVNA) titers and long-term clinical efficacy data to provide a robust foundation on which to reevaluate titer thresholds used to define efficacy for immunobridging purposes for both immunocompetent and immunocompromised (IC) individuals.

The VYD2311 dose modeling strategy contemplated a range of doses for IV up to 4500 mg, doses for IM up to 1000 mg, and doses for SC up to 750 mg. The results of this most recent analysis align well with prior published estimates of relationships between sVNA titers and observed clinical efficacy across multiple COVID-19 mAbs.

All dosing routes and doses assessed yielded modeled efficacy rates that appear to eclipse estimated contemporary rates of COVID-19 vaccine protection for both IC and non-IC individuals. Notably, IM and SC dosing every three months reflected robust efficacy for both IC and non-IC individuals and such routes could simplify and scale administration, improving convenience. Modeling data indicate that IM administration can offer efficacy comparable to high dose IV dosing. Critically, the IM route could eliminate the need for IV infrastructure-enhancing tolerability, accessibility, and convenience. Antiviral titers conferred by VYD2311 via IV to treat active infection are expected to substantially exceed the titers conferred by the control antibody and provide longer suppression of virus.

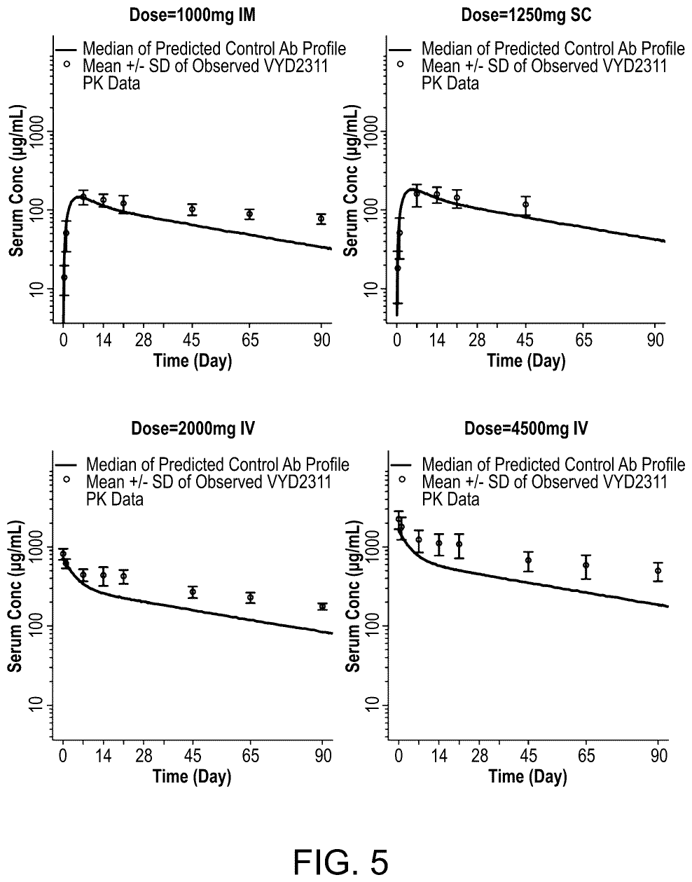

FIG. 5 provides a graphical representation of all actual systemic levels of VYD-2311 achieved (mean+/−SD; dots with error bars) compared to the median prediction (solid line) of the control mAb. At all doses and across all administration routes, VYD2311 appears to have higher achieved levels than the control mAb.

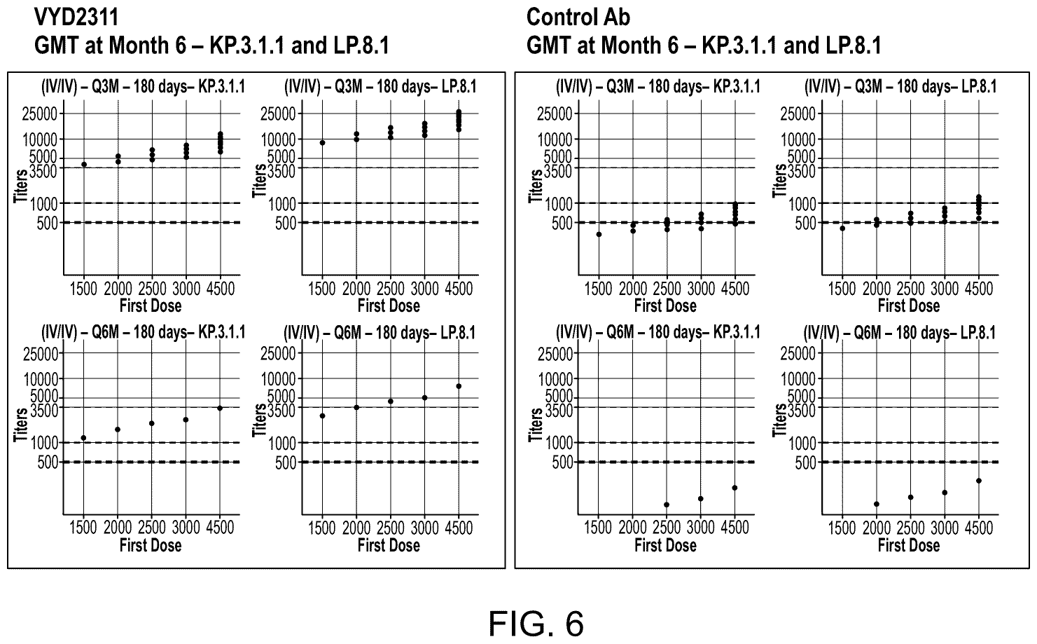

Dose modeling comparing geometric mean titers (GMT) across with IV route of the control mAb versus VYD-2311 at 3 months (one dose) and at 6 months (following two doses administered three months apart) was provided in FIG. 6. These data show that VYD2311 achieves superior GMTs at all doses and dose intervals modelled compared the control mAb.

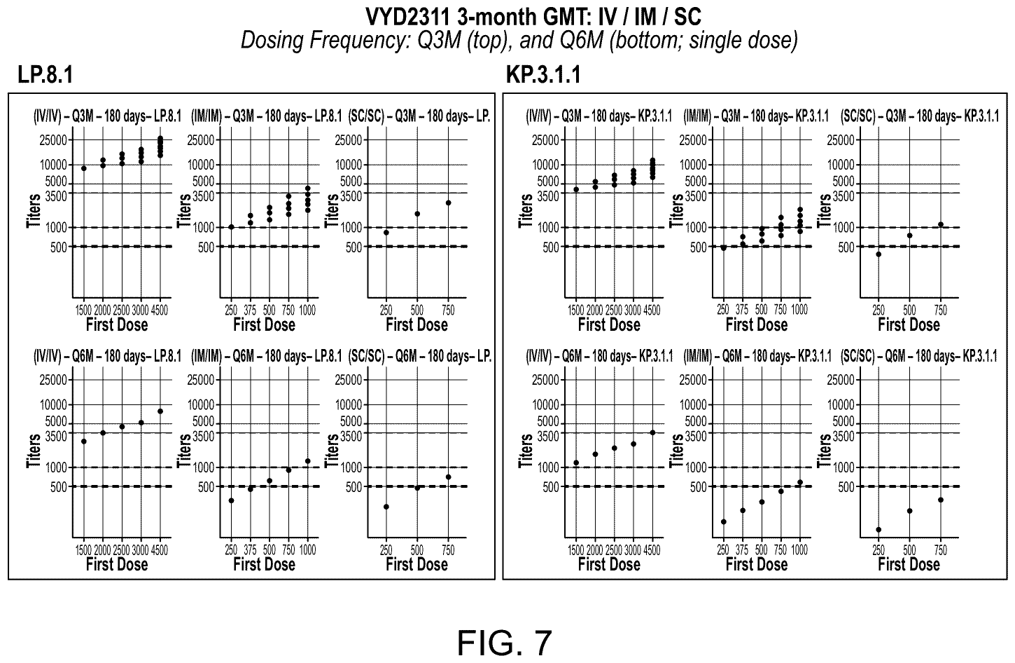

FIG. 7 shows the modelled GMT titers with VYD2311 dosed via IV, IM or SC at the end of 6 months dosed every 3 months (top panel) or every 6 months (bottom panel) for the relevant variants LP.8.1 and KP.3.1.1. These data show that regardless of variant assessed or administration route, the titers achieved are robust and compare favorably to the control mAb.

Phase ½ data, combined with antiviral assessment and COVID-19 antiviral correlate of protection data, support a potential clinical profile for VYD2311 with substantially superior efficacy, safety, and durability to COVID-19 vaccines: stronger protection (70-90%) from symptomatic COVID-19 disease; less frequent (e.g., once- or twice-annual) intramuscular (IM) or subcutaneous (SC) dosing with consistent high protection (as opposed to rapidly waning benefit requiring frequent re-boost); and more favorable safety and tolerability expected to be limited to injection reactions. Further, since VYD2311 is not a vaccine, no activation of subject immune systems.

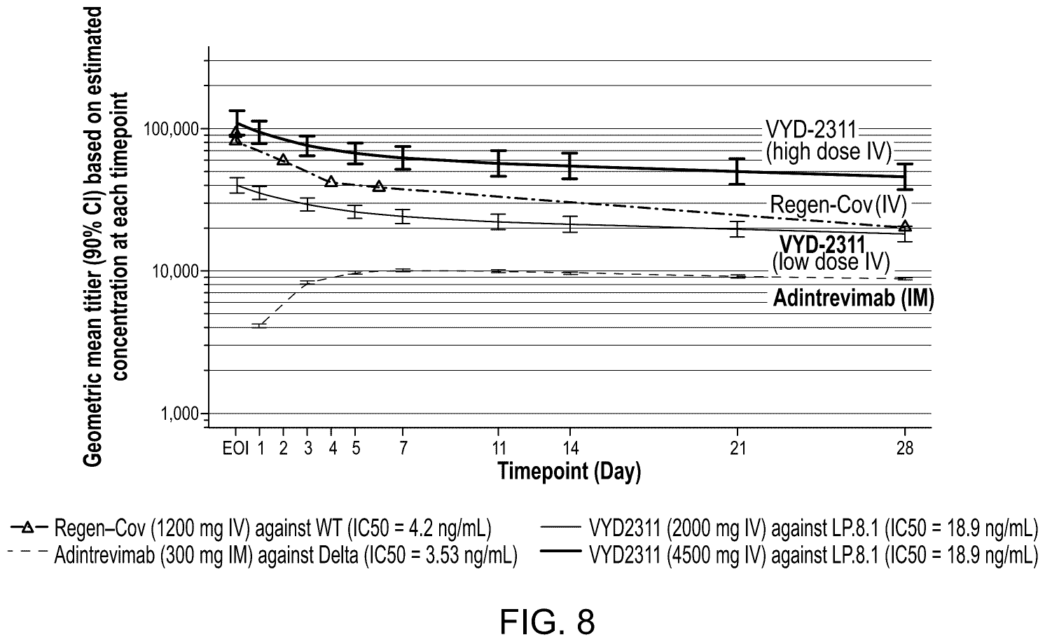

FIG. 8 depicts the serum virus neutralizing antibody (sVNA) titer for VYD2311, and other COVID-19 mAbs. Geometric mean concentrations and 90% confidence interval at each time point for VYD2311 and adintrevimab (ADG20) were summarized with population PK-model-estimated post hoc concentrations of individual subjects in the corresponding Phase 1 clinical study (VYD2311-1-001) and the Phase 3 clinical study (ADG20-PREV-001), respectively. Mean concentrations of the REGEN-CoV were reported in literature (Portal-Celhay C, et al. JAMA Netw Open. 2022; 5(8):e2225411) or per product label. Concentrations were then divided by the corresponding IC50 value of dominant circulating variant when trials were conducted for adintrevimab and REGEN-CoV, or at the present time for VYD2311. As shown in FIG. 8, VYD2311 offers a multi-fold increase in titers at both proposed doses (2000 mg IV; 4500 mg IV) when compared the predicate antibodies, suggesting that VYD2311 has the potential to deploy sufficient antiviral activity rapidly, and to reduce the duration of the symptoms and the likelihood of hospitalization or death of patients, thus offering an opportunity for regulatory pathway to a broader population for both the treatment and/or prevention of COVID-19 disease.

Taken together, these VYD2311 safety, PK, and virology data, along with data from CANOPY Phase 3 clinical trial and other prior mAb pre-exposure prophylaxis studies, predict an attractive clinical protective profile of VYD2311, especially relative to COVID-19 vaccination, including likely highly protective titers of VYD2311 for up to six months or a year or more following a single dose. Specifically, VYD2311 may provide stronger protection (70-90%) from symptomatic COVID-19 disease. Shorter dosing intervals (e.g., quarterly or semi-annual) via either IM injection by a healthcare provider or at home via SC self-administration may be attractive for immunocompromised persons or other populations who may benefit from higher serum virus neutralizing antibody (sVNA) titers and, therefore, higher titer protection, and who also do not wish to interact with healthcare infrastructure to acquire protection from COVID-19. Further, IM administration or higher doses of VYD2311 via IV infusion may be an attractive profile for purposes of delivering maximum possible antiviral activity to patients in need of treating active COVID-19. Finally, VYD2311 exhibits more favorable safety and tolerability than COVID-19 vaccines and, since VYD2311 is not a vaccine, no activation of subject immune systems.

### Example 5. Phase 2 Prevention Study

The prevention study is a single-arm, open-label Phase 2 study to evaluate the calculated serum viral neutralization antibody (sVNA) titer, safety, PK, and immunogenicity of VYD2311 in immunocompetent and immunocompromised adults and adolescents (≥12 years of age and weighing at least 40 kg) at high risk for progression to severe COVID-19.

Approximately 600 participants (300 immunocompromised, 300 with other risk factors for progression to severe COVID-19) are enrolled and receive VYD2311 on Day 1 via IM injection on Day 1 and Month 3. Participants are followed for 9 months after the initial dose. Study visits are conducted on Day 1 and Day 28 and Months 3, 6, and 9, with virtual visits the day after each dose. A subset of participants attends a study visit on Day 10 for additional PK sampling.

The primary efficacy objective is to evaluate protection against symptomatic COVID-19 based on calculated sVNA titers (VYD2311 serum concentration/variant IC50) against relevant SARS-CoV-2 variants after receiving VYD2311. The primary safety objective is to evaluate the safety and tolerability of VYD2311 through 9 months. Secondary endpoints include evaluation of the PK (i.e., serum concentrations of VYD2311) and immunogenicity (incidence of anti-drug antibodies against of VYD2311).

### Inclusion Criteria:

Participants are eligible to be included in the study only if all the following criteria apply:
1. Is an adult aged ≥18 years or an adolescent aged ≥12 to <18 years weighing at least 40 kg at the time of Screening.
2. Tests negative for current SARS-CoV-2 infection by local rapid antigen test or RT-PCR at Day 1 pre-dose. Sites may use an in-house RT-PCR if available.
3. Has immune compromise or other risk factors for progression to severe COVID-19, as described below.
4. Has access to a device (e.g., mobile phone, tablet) enabled to receive study reminders (e.g., SMS text messages).
5. For participants assigned female sex at birth:
   a. Is not of childbearing potential, OR
   b. Is of childbearing potential and practicing adequate contraception for at least 28 days before dosing (Day 1) through 6 months after dosing and has a negative pregnancy test result on Day 1.
6. Provides written documentation of informed consent by signing a current IEC/IRB approved ICF at the time of Screening. A legally authorized representative may be used in cases where inclusion 8 is able to be fulfilled. In the case of adolescents, informed consent/assent must also be obtained as required by local guidelines.
7. Is able to understand the study requirement/procedures (if applicable, with assistance by a caregiver, surrogate, or legally authorized representative) based on the assessment of the Investigator and willing to adhere to all protocol requirements.

### Exclusion Criteria:

Participants are excluded from the study if any of the following criteria apply:
1. Previously received VYD2311 or pemivibart (VYD222) within 12 months before enrollment on Day 1.
2. Prior receipt of convalescent plasma or a mAb to SARS-CoV-2 active against currently circulating variants, including in the setting of a clinical trial, within 6 months before enrollment on Day 1.
3. Prior known or suspected SARS-CoV-2 infection within 28 days before enrollment on Day 1.
4. Exposure to someone with known or suspected SARS-CoV-2 infection in the 5 days before enrollment on Day 1.
5. Is acutely ill or has any of the following symptoms suggestive of infection, in the opinion of the Investigator:

| | |
|---|---|
| Fever ≥38° C. (≥100.4° F.) | Shortness of breath/ |
| | difficulty breathing |
| Chills (shivering | Cough |
| Fatigue (low energy of tiredness) | Muscle or body |
| Headache | Loss of taste |
| Loss of smell | Sore throat |
| Congestion (stuffy or runny nose) | Nausea |
| Vomiting | Diarrhea |

6. Received any investigational product within 30 days or 5 half-lives (whichever is longer) before the day of enrollment.
7. Received or plans to receive any vaccine within 28 days before or after dosing on Day 1 (except for seasonal influenza and COVID-19 vaccines, which are not permitted within 14 days before or after dosing on Day 1).
8. Known or suspected allergy/sensitivity or hypersensitivity/anaphylactic reactions or other serious adverse reactions to the study drug, including excipients, or other monoclonal antibodies.
9. Pregnant, as confirmed with a positive pregnancy test on the day of dosing (Day 1), or seeking pregnancy during the time of the study, or breastfeeding.
10. Known clinically significant bleeding disorder (eg, factor deficiency, coagulopathy, or platelet disorder), or prior history of significant bleeding or bruising following IM injections or venipuncture. Abnormal coagulation labs or use of anticoagulant medication are not exclusionary in the absence of clinical findings.
11. Any serious concomitant systemic disease, condition, or disorder that, in the opinion of the Investigator, may lead to hospitalization or death within the study period, confound the results of the study, or confer an additional risk to the participant by their participation in the study.
12. Is or has an immediate family member (eg, spouse, sibling, child, guardian/LAR, parent) who is an Investigator or site/Sponsor employee directly involved with the study.

### Risk Factors for Severe COVID-19

The following are examples of conditions that place an individual at increased risk for progression to severe COVID-19:

### Immunocompromised Conditions

Active treatment for solid tumor and hematologic malignancies
Hematologic malignancies associated with poor response to COVID-19 vaccines regardless of current treatment status (eg, chronic lymphocytic leukemia, non-Hodgin lymphoma, multiple myeloma, acute leukemia)
Receipt of solid-organ transplant or an islet transplant and taking immunosuppressive therapy
Receipt of chimeric antigen receptor (CAR)-T-cell or hematopoietic stem cell transplant (within 2 years of transplantation of taking immunosuppressive therapy)
Moderate or severe primary immunodeficiency (eg, common variable immunodeficiency disease, severe combined immunodeficiency, DiGeorge syndrome, Wiskott-Aldrich syndrome)
Advanced or untreated HIV infection (people with HIV and CD4 cell counts <200/mm³, history of an AIDS-defining illness without immune reconstitution, or clinical manifestations of symptomatic HIV)
Active treatment with high-dose corticosteroids (ie, ≥20 mg prednisone or equivalent per day when administered for ≥2 weeks), alkylating agents, antimetabolites, transplant-related immunosuppressive drugs, cancer chemotherapeutic agents classified as severely immunosuppressive, and biologic agents that are immunosuppressive or immunomodulatory (eg, B-cell depleting agents)

### Other Risk Factors for Progression to Severe COVID-19

Age ≥65 years
Obesity with a BMI≥30 kg/m2 or for adolescents 12 to 17 years BMI≥95th percentage for age and gender based on CDC growth charts
Cerebrovascular disease or stroke, which affects blood flow to the brain
Chronic kidney disease, excluding dialysis
Chronic liver disease, including alcohol-related liver disease, non-alcoholic fatty liver disease, autoimmune hepatitis, and cirrhosis
Chronic lung disease, including moderate to severe asthma, bronchiectasis, chronic obstructive pulmonary disease (including emphysema and chronic bronchitis), damaged or scarred lung tissue (interstitial lung disease including idiopathic pulmonary fibrosis), pulmonary embolism or pulmonary hypertension
Cystic fibrosis
Diabetes (Type 1 or Type 2)
Down syndrome
Heart conditions, including heart failure, coronary artery disease, cardiomyopathies, and hypertension (requiring at least 1 medication prescribed or recommended)
Hemoglobin blood disorders, including sickle cell disease and thalassemia
HIV infection (people with HIV and CD4 cell counts>200/mm3, without a history of an AIDS-defining illness, and without clinical manifestations of symptomatic HIV)
Neurologic conditions, including dementia and Parkinson's Disease

### Primary Immuno-Bridging Analysis

The primary analysis evaluates the ratio of the calculated sVNA geometric median titers (GMT) against a relevant variant at Day 28 post-VYD2311 administration among participants in the PK Full Analysis Set (PK-FAS). PK-FAS includes all participants (pooled across immune compromised and immune competent groups) who receive a full dose of study drug at the initial dosing and have a quantifiable serum concentration result (≥LLOQ) at Day 28. The GMT for VYD2311 are evaluated against a Day 28 Titer Target, extrapolated from a protective threshold of 500 at six months post-initial dose. Non-inferiority (establishing strict immunobinding) is concluded if the lower bound of the two-sided 90% confidence interval for the GMT ratio exceeds 0.8.

The statistical analysis plan is finalized prior to any interim analysis. The primary efficacy objective is to evaluate protection against symptomatic COVID-19 based on sVNA titers against SARS-CoV-2 following VYD2311 injection by immunobridging to a target selected based on historical data analysis from the CANOPY study. The immunobridging approach compares calculated sVNA titers against relevant variants using serum VYD2311 concentrations measured on Day 28 (serum VYD2311 concentration/variant IC50) to a reference Day 28 sVNA titer target expected to remain above a target of 500 for a minimum of 3 months based on PK modeling.

The null and alternative hypotheses for the primary efficacy analysis are the following:
Ho: GMTv/T≤0.80
H1: GMTv/T>0.80, where GMTv is the calculated sVNA GMT against a relevant variant at Day 28 following VYD2311 and T is the extrapolated Day 28 sVNA titer target associated with clinical efficacy for 3 months. Immunobridging is demonstrated if the lower limit of the 2-sided 90% CI of the ratio between the calculated sVNA GMT against a relevant variant at Day 28 following VYD2311 and the extrapolated Day 28 titer target is greater than 0.8.

### Example 6. Phase 2 Treatment Study

The treatment study is a single-arm, open-label Phase 2 study to evaluate the calculated serum viral neutralization titer (sVNA), safety, PK, and immunogenicity of VYD2311 in immunocompetent and immunocompromised adults and adolescents (≥12 years of age and weighing at least 40 kg) at high risk for progression to severe COVID-19. The study is similar in design to the prevention study (Example 5), with the exception of route of administration, dosing scheme, and follow-up visits.

Approximately 600 participants (300 immunocompromised, 300 with other risk factors for progression to severe COVID-19) are enrolled and receive VYD2311 on Day 1 via IV infusion. On Day 1, participants are pre-medicated with cetirizine 10 mg per oral approximately 30 minutes prior to infusion and then receive VYD2311 IV. Redosing are planned at an appropriate timepoint based on PK modelling and calculated pseudovirus neutralization activity against relevant variants at the time of the study.

Initiation of the study is triggered only after participants in the Phase 1 study have been followed through the Day 14 visit. The primary efficacy objective is to evaluate protection against symptomatic COVID-19 following a single dose of VYD2311. The primary efficacy endpoint is sVNA titers against relevant SARS-CoV-2 variant(s) after receiving VYD2311, calculated by VYD2311 serum concentration/variant IC50. The primary safety objective is to evaluate the safety and tolerability of VYD2311 through 6 months. The primary safety endpoint is the incidence of AEs. Key secondary endpoints include PK (i.e., serum concentrations of VYD2311) and ADA assessments and RT-PCR-confirmed symptomatic COVID-19.

### Example 7. Phase 3 Prevention Study

The confirmatory prevention study is a randomized, double-blind, placebo-controlled study of VYD2311 in adults and adolescents with or without immune compromise. Approximately 1500 participants are randomized 2:1 to receive either VYD2311 (N=1000) or placebo (N=500) via IM administration on Day 1, with redosing at Month 3. Participants are followed for 12 months.

The primary efficacy objective is to evaluate the clinical efficacy of VYD2311 compared with placebo in the prevention of RT-PCR-confirmed symptomatic COVID-19, measured through 3, 6, 9 and 12 months. The primary safety objective is to evaluate the safety and tolerability of VYD2311. The primary safety endpoint is the incidence of AEs. Secondary endpoints include calculated sVNA titers against relevant SARS-CoV-2 variants, serum concentrations of VYD2311 over time, and anti-drug antibodies against VYD2311.

### Example 8. Phase 3 Treatment Study

The phase 3 treatment study is a randomized, double-blind, placebo-controlled study of VYD2311 in adults and adolescents with or without immune compromise. Participants are randomized 2:1 to receive either VYD2311 or placebo via IV administration.

The primary objective is to confirm safety and development of resistance to VYD2311 in participants with confirmed symptomatic COVID-19 compared with placebo to support transition to full BLA approval following completion of the study.

### Example 9. Additional Antibodies with Improved Binding Affinity and Neutralization Efficacy

This Example provides additional antibodies, ADI-90031, ADI-90032, ADI-90033, and ADI-90035, with improved binding affinity and neutralizing potency against selected Omicron variants as described in Example 1. Sequences for the antibodies are included in Table 13.

ADI-90031 differs from VYD2311 by 1 amino acid in the VH CDR1 region, 2 amino acids in the VL CDR1 region and 1 amino acid in the VL CDR3 region.

ADI-90032 differs from VYD2311 by 1 amino acid in the VL CDR1 region, 1 amino acid in the VL CDR2 region, and 1 amino acid in the VL CDR3 region.

ADI-90033 differs from VYD2311 by 1 amino acid in the VL CDR2 region and 1 amino acid in the VL CDR3 region.

ADI-90035 differs from VYD2311 by 1 amino acid in the VH CDR1 region, 2 amino acids in the VH CDR2 region, 1 amino acid in the VH CDR3 region, 1 amino acid in the VL CDR1 region, 1 amino acid in the VL CDR2 region, and 1 amino acid in the VL CDR3 region.

Binding affinities for VYD2311, ADI-90031, ADI-90032 and ADI-90033 were determined for XBB.1.5 and BA.2.86 RBD and Spike trimer using the Carterra LSA SPR instrument at WuXi biologics, as described in Example 1. As shown in Table 11 below, ADI-90031, ADI-90032 and ADI-90033 have improved binding against the Omicron variants, XBB.1.5 and BA.2.86, in the sub-nanomolar range, similar to VYD2311.

TABLE 11
Binding Affinities for ADI-90031, ADI-90032 and ADI-90033

| | RBD Omicron | RBD BA.2.86 |
|---|---|---|
| Ligand | Mean K_{D} (M) | Mean K_{D} (M) |
| VYD2311 | 3.7E−10 | 1.1E−09 |
| ADI-90031 | 3.7E−10 | 9.0E−10 |
| ADI-90032 | 5.3E−10 | 1.5E−09 |
| ADI-90033 | 2.7E−10 | 8.5E−10 |

The neutralization activity of ADI-90031, ADI-90032, ADI-90033, and ADI-90035 against SARS CoV-2 pseudovirus was also evaluated in an MLV-based pseudovirus neutralizing assay. Briefly, dilutions of each antibody were prepared in Hela-ACE2 media, with starting concentrations of 5 μg/mL or 20 μg/mL followed by 3-fold serial dilutions in 96-well plates. Dilutions were mixed 1:1 with RLU-normalized pseudovirus and incubated at 37 C for 1 hour. Samples were then transferred to Hela-ACE2 target cells and incubated for 48 hours. Luciferase was measured using BrightGlo reagent (Promega) and a VarioSkan Lux microplate reader. Percent neutralization was measured using the formula 1-[(RLU of test well)/(Average RLU of pseudovirus only wells)]×100. Neutralization curves and IC50 values were generated in GraphPad Prism version 10.4.0 using a 4PL non-linear regression with constraints of Bottom=0, Top=100, and HillSlope>0. As shown in Table 12, ADI-90031, ADI-90032, ADI-90033, and ADI-90035 demonstrate broad and potent in vitro neutralizing activity against all pseudotyped SARS-CoV-2 variants evaluated, including Omicron sublineages XBB.1.5, XBB.1.5.10, BA.2.86, HK.3, HV.1, FL.15.1.1, JN.1, XBB.1.5.70 and JN.4.

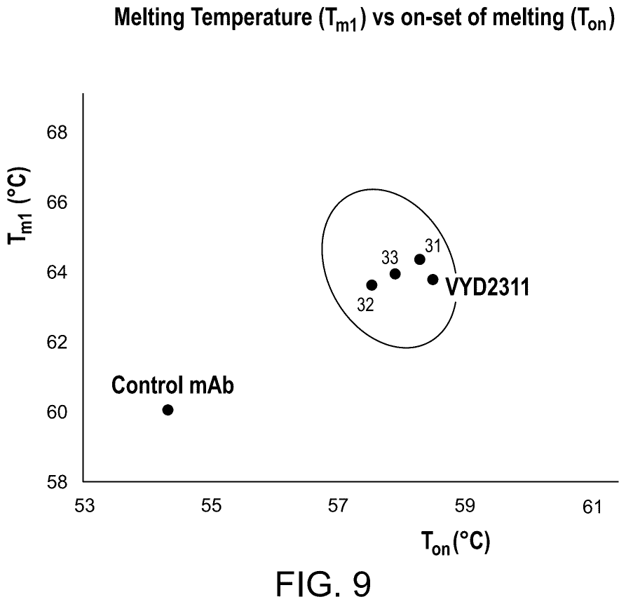

The developability profile of ADI-90031, ADI-90032, ADI-90033 was also analyzed, alongside the control antibody as a comparator antibody, as described in Example 1. As shown in FIG. 9, ADI-90031, ADI-90032 and ADI-90033 had a comparably higher T_{M} and Tₒₙ (the onset of melting) as VYD2311, which leads to improved shelf life and half-life. These results suggest that like VYD2311, ADI-90031, ADI-90032, and ADI-90033 had more favorable thermal stability properties in vitro and, thus, are more stable than previous antibodies.

TABLE 12
In vitro Neutralization Activity

| | Antibody | ADI-90031 | ADI-90032 | ADI-90033 | ADI-90035 |
|---|---|---|---|---|---|
| XBB.1.5 | IC₅₀ | 0.0485 | 0.0903 | 0.0707 | 0.097 |
| (μg/mL) | Std Dev | 2.2 | 3.01 | 2.82 | 2.48 |
| XBB.1.5.10 | IC₅₀ | 0.0478 | 0.00975 | 0.0302 | 0.0348 |
| (μg/mL) | Std Dev | 7.53 | 1.48 | 3.4 | 1.08 |
| BA.2.86 | IC₅₀ | <7.48E−03 | 0.00959 | 0.00831 | 0.0425 |
| (μg/mL) | Std Dev | 1.87 | 1.96 | 1.89 | 1.52 |
| HK.3 | IC₅₀ | 0.0113 | 0.0064 | 0.0163 | |
| (μg/mL) | Std Dev | | | | |
| HV.1 | IC₅₀ | 0.00685 | 0.00726 | 0.00918 | |
| (μg/mL) | Std Dev | 1.11 | 1.22 | 1.82 | |
| FL.15.1.1 | IC₅₀ | 0.0126 | 0.00956 | 0.0115 | |
| (μg/mL) | Std Dev | | | | |
| JN.1 | IC₅₀ | 0.0162 | 0.0206 | 0.013 | |
| (μg/mL) | Std Dev | 1.39 | 1.15 | 2.01 | |
| XBB.1.5.70 | IC₅₀ | 0.0138 | 0.0106 | 0.0119 | |
| (μg/mL) | Std Dev | 1.29 | 1.21 | 1.28 | |
| JN.4 | IC₅₀ | 0.0124 | 0.0178 | 0.00761 | |
| (μg/mL) | Std Dev | | | | |

TABLE 13
Antibody Sequences

| Antibody | VYD2311 | SEQ ID NO. |
|---|---|---|
| VH Germline | VH3-21 | |
| VH FR1 | EVQLVESGGGLVKPGGSLRLSCAASG | 11 |
| VH CDR1 | FEFGSYEMN | 12 |
| VH FR2 | WVRQAPGKGLEWVS | 13 |
| VH CDR2 | SISEDGYTTYYPDSLKG | 14 |
| VH FR3 | RFTISRDSAKNSLYLQMNSLRADDTAVYYC | 15 |
| VH CDR3 | ARDFGGDTNWAGTGFTY | 16 |
| VH FR4 | WGQGTLVTVSS | 17 |
| VH DNA | GAAGTACAGCTGGTGGAGTCAGGAGGAGGTCTGGTCA | 18 |
| | AACCAGGAGGATCACTTAGGCTGAGCTGCGCTGCCTCT | |
| | GGCTTTGAATTTGGGTCTTATGAAATGAATTGGGTTCG | |
| | ACAAGCACCTGGAAAGGGCCTCGAATGGGTTTCCTCTA | |
| | TCTCTGAAGATGGATACACAACCTATTACCCCGACTCA | |
| | CTCAAAGGCCGATTCACAATTTCCCGAGATTCCGCTAA | |
| | AAACTCACTGTATCTGCAAATGAATAGCCTAAGGGCC | |
| | GATGACACAGCCGTGTACTATTGTGCCCGCGACTTTGG | |
| | CGGGGACACTAATTGGGCTGGAACCGGCTTTACTTACT | |
| | GGGGACAGGGAACACTCGTGACCGTTTCAAGC | |
| VH | EVQLVESGGGLVKPGGSLRLSCAASGFEFGSYEMNWVR | 19 |
| | QAPGKGLEWVSSISEDGYTTYYPDSLKGRFTISRDSAKNS | |
| | LYLQMNSLRADDTAVYYCARDFGGDTNWAGTGFTYWG | |
| | QGTLVTVSS | |
| VL Germline | VL1-40 | |
| VL FR1 | QSVLTQPPSVSGAPGQRITISC | 21 |
| VL CDR1 | EGSSSNIGAGYDVH | 22 |
| VL FR2 | WYQQLPGTAPKLLIY | 23 |
| VL CDR2 | GSSVRNY | 24 |
| VL FR3 | GVPDRFSGSKSGTSASLAITGLQAEDEADYYC | 25 |
| VL CDR3 | QSYDSDLGILYT | 26 |
| VL FR4 | FGTGTKVTVL | 27 |
| VL DNA | CAGAGCGTTTTAACCCAACCGCCAAGCGTCTCTGGTGC | 28 |
| | ACCGGGTCAAAGAATCACTATCTCCTGTGAAGGATCCT | |
| | CTTCAAATATTGGGGCTGGCTATGATGTGCACTGGTAT | |
| | CAGCAACTACCTGGGACCGCTCCTAAGCTGCTGATTTA | |
| | TGGAAGTAGTGTTCGCAACTATGGAGTGCCGGACAGA | |
| | TTCAGTGGATCCAAATCTGGAACCAGCGCCTCCCTCGC | |
| | TATCACCGGCCTGCAAGCCGAGGATGAGGCCGACTAC | |
| | TACTGCCAAAGTTACGACTCTGACTTGGGCATCCTGTA | |
| | CACCTTCGGGACCGGCACCAAGGTGACTGTGTTG | |
| VL | QSVLTQPPSVSGAPGQRITISCEGSSSNIGAGYDVHWYQQ | 29 |
| | LPGTAPKLLIYGSSVRNYGVPDRFSGSKSGTSASLAITGLQ | |
| | AEDEADYYCQSYDSDLGILYTFGTGTKVTVL | |
| Antibody | Control Antibody | SEQ ID NO. |
| VH Germline | VH3-21 | |
| VH FR1 | EVQLVESGGGLVKPGGSLRLSCAASG | 31 |
| VH CDR1 | FTFGSYEMN | 32 |
| VH FR2 | WVRQAPGKGLEWVS | 33 |
| VH CDR2 | SISEDGYSTYYPDSLKG | 34 |
| VH FR3 | RFTISRDSAKNSLYLQMNSLRADDTAVYYC | 35 |
| VH CDR3 | ARDFGGDTAWAGTGFTY | 36 |
| VH FR4 | WGQGTLVTVSS | 37 |
| VH DNA | GAGGTGCAGTTAGTCGAAAGCGGTGGAGGACTTGTTA | 38 |
| | AACCAGGAGGGTCACTAAGGTTGAGTTGCGCGGCTTC | |
| | AGGATTCACCTTCGGATCATACGAAATGAACTGGGTTC | |
| | GTCAAGCGCCGGGAAAAGGCTTGGAGTGGGTCAGTAG | |
| | TATCTCAGAGGATGGATACAGCACGTATTATCCTGATT | |
| | CCTTGAAAGGTCGTTTTACAATAAGTAGGGATTCCGCT | |
| | AAAAACTCATTGTATCTTCAAATGAACTCCTTGAGGGC | |
| | TGACGACACGGCTGTGTATTACTGCGCGAGAGACTTTG | |
| | GTGGCGACACTGCGTGGGCGGGAACGGGTTTTACCTA | |
| | CTGGGGACAGGGCACCCTTGTTACAGTGTCAAGT | |
| VH | EVQLVESGGGLVKPGGSLRLSCAASGFTFGSYEMNWVR | 39 |
| | QAPGKGLEWVSSISEDGYSTYYPDSLKGRFTISRDSAKNS | |
| | LYLQMNSLRADDTAVYYCARDFGGDTAWAGTGFTYWG | |
| | QGTLVTVSS | |
| VL Germline | VL1-40 | |
| VL FR1 | QSVLTQPPSVSGAPGQRITISC | 41 |
| VL CDR1 | TGSSSNIGAGYDVH | 42 |
| VL FR2 | WYQQLPGTAPKLLIY | 43 |
| VL CDR2 | GSSSRNY | 44 |
| VL FR3 | GVPDRFSGSKSGTSASLAITGLQAEDEADYYC | 45 |
| VL CDR3 | QSYDSDLGVLYT | 46 |
| VL FR4 | FGTGTKVTVL | 47 |
| VL DNA | CAATCTGTACTTACACAGCCTCCCAGCGTGAGCGGTGC | 48 |
| | TCCAGGACAGAGGATCACTATTTCCTGTACTGGCAGTT | |
| | CATCCAATATTGGTGCAGGATATGACGTTCATTGGTAC | |
| | CAACAACTTCCGGGAACCGCCCCAAAACTTTTAATCTA | |
| | TGGCAGCAGTAGTCGTAATTATGGTGTGCCTGATAGGT | |
| | TCTCCGGCTCTAAAAGCGGTACCAGTGCGAGTTTAGCC | |
| | ATCACAGGGCTACAAGCCGAGGATGAGGCCGATTATT | |
| | ATTGTCAAAGCTACGATAGCGATCTAGGCGTTTTGTAC | |
| | ACCTTCGGTACAGGCACGAAAGTCACTGTTCTT | |
| VL | QSVLTQPPSVSGAPGQRITISCTGSSSNIGAGYDVHWYQQ | 49 |
| | LPGTAPKLLIYGSSSRNYGVPDRFSGSKSGTSASLAITGLQ | |
| | AEDEADYYCQSYDSDLGVLYTFGTGTKVTVL | |
| Antibody | ADG20 | SEQ ID NO. |
| VH Germline | VH3-21 | |
| VH FR1 | EVQLVESGGGLVKPGGSLRLSCAASG | 51 |
| VH CDR1 | FTFSSYYMN | 52 |
| VH FR2 | WVRQAPGKGLEWVS | 53 |
| VH CDR2 | SISEDGYSTYYPDSLKG | 54 |
| VH FR3 | RFTISRDSAKNSLYLQMNSLRADDTAVYYC | 55 |
| VH CDR3 | ARDFSGHTAWAGTGFEY | 56 |
| VH FR4 | WGQGTLVTVSS | 57 |
| VH DNA | GAGGTGCAGCTGGTGGAGTCTGGGGGAGGCCTGGTCA | 58 |
| | AGCCTGGGGGGTCCCTGAGACTGTCCTGTGCAGCCTCT | |
| | GGATTCACCTTCAGTAGCTATTATATGAACTGGGTCAG | |
| | GCAGGCTCCAGGGAAGGGGCTGGAGTGGGTCTCATCC | |
| | ATTAGTGAGGACGGATACAGCACCTACTACCCCGACTC | |
| | ACTGAAGGGCAGATTCACCATCTCCAGAGACAGCGCC | |
| | AAGAACTCACTGTATCTGCAAATGAACAGCCTGAGAG | |
| | CCGATGACACGGCTGTGTATTACTGTGCCAGAGATTTT | |
| | TCAGGCCACACGGCGTGGGCAGGAACGGGATTTGAGT | |
| | ACTGGGGCCAAGGAACCCTGGTCACCGTCTCCTCA | |
| VH | EVQLVESGGGLVKPGGSLRLSCAASGFTFSSYYMNWVR | 59 |
| | QAPGKGLEWVSSISEDGYSTYYPDSLKGRFTISRDSAKNS | |
| | LYLQMNSLRADDTAVYYCARDFSGHTAWAGTGFEYWG | |
| | QGTLVTVSS | |
| VL Germline | | |
| VL FR1 | QSVLTQPPSVSGAPGQRITISC | 61 |
| VL CDR1 | TGSSSNIGAGYDVH | 62 |
| VL FR2 | WYQQLPGTAPKLLIY | 63 |
| VL CDR2 | GSSSRNS | 64 |
| VL FR3 | GVPDRFSGSKSGTSASLAITGLQAEDEADYYC | 65 |
| VL CDR3 | QSYDSSLSVLYT | 66 |
| VL FR4 | FGTGTKVTVL | 67 |
| VL DNA | CAGTCTGTGCTGACGCAGCCGCCCTCAGTGTCTGGGGC | 68 |
| | CCCAGGGCAGAGGATCACCATCTCCTGCACTGGGAGC | |
| | AGCTCCAACATCGGGGCAGGTTATGATGTACACTGGTA | |
| | CCAGCAGCTTCCAGGAACAGCCCCCAAACTTCTGATCT | |
| | ATGGTTCTAGCTCCAGGAACTCAGGGGTCCCTGACAGG | |
| | TTCTCTGGCTCCAAGTCTGGCACCTCAGCCTCCCTGGC | |
| | CATCACTGGGCTTCAGGCTGAGGATGAGGCTGATTATT | |
| | ACTGCCAAAGTTATGACTCTAGTCTGTCCGTCTTGTAT | |
| | ACGTTCGGAACTGGGACCAAGGTCACCGTCCTG | |
| VL | QSVLTQPPSVSGAPGQRITISCTGSSSNIGAGYDVHWYQQ | 69 |
| | LPGTAPKLLIYGSSSRNSGVPDRESGSKSGTSASLAITGLQ | |
| | AEDEADYYCQSYDSSLSVLYTFGTGTKVTVL | |
| Antibody | ADI-90031 | SEQ ID NO. |
| VH Germline | VH3-21 | |
| VH FR1 | EVQLVESGGGLVKPGGSLRLSCAASG | 71 |
| VH CDR1 | FDFGSYEMN | 72 |
| VH FR2 | WVRQAPGKGLEWVS | 73 |
| VH CDR2 | SISEDGYTTYYPDSLKG | 74 |
| VH FR3 | RFTISRDSAKNSLYLQMNSLRADDTAVYYC | 75 |
| VH CDR3 | ARDFGGDTNWAGTGFTY | 76 |
| VH FR4 | WGQGTLVTVSS | 77 |
| VH DNA | GAGGTGCAGTTAGTCGAAAGCGGTGGAGGACTTGTTA | 78 |
| | AACCAGGAGGGTCACTAAGGTTGAGTTGCGCGGCTTC | |
| | AGGATTCGATTTCGGATCATACGAAATGAACTGGGTTC | |
| | GTCAAGCGCCGGGAAAAGGCTTGGAGTGGGTCAGTAG | |
| | TATCTCAGAGGATGGATACACGACGTATTATCCTGATT | |
| | CCTTGAAAGGTCGTTTTACAATAAGTAGGGATTCCGCT | |
| | AAAAACTCATTGTATCTTCAAATGAACTCCTTGAGGGC | |
| | TGACGACACGGCTGTGTATTACTGCGCGAGAGACTTTG | |
| | GTGGCGACACTAATTGGGCGGGAACGGGTTTTACCTAC | |
| | TGGGGACAGGGCACCCTTGTTACAGTGTCAAGT | |
| VH | EVQLVESGGGLVKPGGSLRLSCAASGFDFGSYEMNWVR | 79 |
| | QAPGKGLEWVSSISEDGYTTYYPDSLKGRFTISRDSAKNS | |
| | LYLQMNSLRADDTAVYYCARDFGGDTNWAGTGFTYWG | |
| | QGTLVTVSS | |
| VL Germline | VL1-40 | |
| VL FR1 | QSVLTQPPSVSGAPGQRITISC | 81 |
| VL CDR1 | TGTSSNIGAGYDVH | 82 |
| VL FR2 | WYQQLPGTAPKLLIY | 83 |
| VL CDR2 | GSSVRNY | 84 |
| VL FR3 | GVPDRFSGSKSGTSASLAITGLQAEDEADYYC | 85 |
| VL CDR3 | QSYDSDLGVLYT | 86 |
| VL FR4 | FGTGTKVTVL | 87 |
| VL DNA | CAATCTGTACTTACACAGCCTCCCAGCGTGAGCGGTGC | 88 |
| | TCCAGGACAGAGGATCACTATTTCCTGTACTGGCACAT | |
| | CATCCAATATTGGTGCAGGATATGACGTTCATTGGTAC | |
| | CAACAACTTCCGGGAACCGCCCCAAAACTTTTAATCTA | |
| | TGGCAGCAGTGTTCGTAATTATGGTGTGCCTGATAGGT | |
| | TCTCCGGCTCTAAAAGCGGTACCAGTGCGAGTTTAGCC | |
| | ATCACAGGGCTACAAGCCGAGGATGAGGCCGATTATT | |
| | ATTGTCAAAGCTACGATAGCGATCTAGGCGTTTTGTAC | |
| | ACCTTCGGTACAGGCACGAAAGTCACTGTTCTT | |
| VL | QSVLTQPPSVSGAPGQRITISCTGTSSNIGAGYDVHWYQQ | 89 |
| | LPGTAPKLLIYGSSVRNYGVPDRESGSKSGTSASLAITGLQ | |
| | AEDEADYYCQSYDSDLGVLYTFGTGTKVTVL | |
| Antibody | ADI-90032 | SEQ ID NO. |
| VH Germline | VH3-21 | |
| VH FR1 | EVQLVESGGGLVKPGGSLRLSCAASG | 91 |
| VH CDR1 | FEFGSYEMN | 92 |
| VH FR2 | WVRQAPGKGLEWVS | 93 |
| VH CDR2 | SISEDGYTTYYPDSLKG | 94 |
| VH FR3 | RFTISRDSAKNSLYLQMNSLRADDTAVYYC | 95 |
| VH CDR3 | ARDFGGDTNWAGTGFTY | 96 |
| VH FR4 | WGQGTLVTVSS | 97 |
| VH DNA | GAGGTGCAGTTAGTCGAAAGCGGTGGAGGACTTGTTA | 98 |
| | AACCAGGAGGGTCACTAAGGTTGAGTTGCGCGGCTTC | |
| | AGGATTCGAGTTCGGATCATACGAAATGAACTGGGTTC | |
| | GTCAAGCGCCGGGAAAAGGCTTGGAGTGGGTCAGTAG | |
| | TATCTCAGAGGATGGATACACGACGTATTATCCTGATT | |
| | CCTTGAAAGGTCGTTTTACAATAAGTAGGGATTCCGCT | |
| | AAAAACTCATTGTATCTTCAAATGAACTCCTTGAGGGC | |
| | TGACGACACGGCTGTGTATTACTGCGCGAGAGACTTTG | |
| | GTGGCGACACTAATTGGGCGGGAACGGGTTTTACCTAC | |
| | TGGGGACAGGGCACCCTTGTTACAGTGTCAAGT | |
| VH | EVQLVESGGGLVKPGGSLRLSCAASGFEFGSYEMNWVR | 99 |
| | QAPGKGLEWVSSISEDGYTTYYPDSLKGRFTISRDSAKNS | |
| | LYLQMNSLRADDTAVYYCARDFGGDTNWAGTGFTYWG | |
| | QGTLVTVSS | |
| VL Germline | VL1-40 | |
| VL FR1 | QSVLTQPPSVSGAPGQRITISC | 101 |
| VL CDR1 | TGSSSNIGAGYDVH | 102 |
| VL FR2 | WYQQLPGTAPKLLIY | 103 |
| VL CDR2 | GSSLRNY | 104 |
| VL FR3 | GVPDRFSGSKSGTSASLAITGLQAEDEADYYC | 105 |
| VL CDR3 | QSYDSDLGVLYT | 106 |
| VL FR4 | FGTGTKVTVL | 107 |
| VL DNA | CAATCTGTACTTACACAGCCTCCCAGCGTGAGCGGTGC | 108 |
| | TCCAGGACAGAGGATCACTATTTCCTGTACTGGCAGTT | |
| | CATCCAATATTGGTGCAGGATATGACGTTCATTGGTAC | |
| | CAACAACTTCCGGGAACCGCCCCAAAACTTTTAATCTA | |
| | TGGCAGCAGTCTGCGTAATTATGGTGTGCCTGATAGGT | |
| | TCTCCGGCTCTAAAAGCGGTACCAGTGCGAGTTTAGCC | |
| | ATCACAGGGCTACAAGCCGAGGATGAGGCCGATTATT | |
| | ATTGTCAAAGCTACGATAGCGATCTAGGCGTTTTGTAC | |
| | ACCTTCGGTACAGGCACGAAAGTCACTGTTCTT | |
| VL | QSVLTQPPSVSGAPGQRITISCTGSSSNIGAGYDVHWYQQ | 109 |
| | LPGTAPKLLIYGSSLRNYGVPDRFSGSKSGTSASLAITGLQ | |
| | AEDEADYYCQSYDSDLGVLYTFGTGTKVTVL | |
| Antibody | ADI-90033 | SEQ ID NO. |
| VH Germline | VH3-21 | |
| VH FR1 | EVQLVESGGGLVKPGGSLRLSCAASG | 111 |
| VH CDR1 | FEFGSYEMN | 112 |
| VH FR2 | WVRQAPGKGLEWVS | 113 |
| VH CDR2 | SISEDGYTTYYPDSLKG | 114 |
| VH FR3 | RFTISRDSAKNSLYLQMNSLRADDTAVYYC | 115 |
| VH CDR3 | ARDFGGDTNWAGTGFTY | 116 |
| VH FR4 | WGQGTLVTVSS | 117 |
| VH DNA | GAGGTGCAGTTAGTCGAAAGCGGTGGAGGACTTGTTA | 118 |
| | AACCAGGAGGGTCACTAAGGTTGAGTTGCGCGGCTTC | |
| | AGGATTCGAGTTCGGATCATACGAAATGAACTGGGTTC | |
| | GTCAAGCGCCGGGAAAAGGCTTGGAGTGGGTCAGTAG | |
| | TATCTCAGAGGATGGATACACGACGTATTATCCTGATT | |
| | CCTTGAAAGGTCGTTTTACAATAAGTAGGGATTCCGCT | |
| | AAAAACTCATTGTATCTTCAAATGAACTCCTTGAGGGC | |
| | TGACGACACGGCTGTGTATTACTGCGCGAGAGACTTTG | |
| | GTGGCGACACTAATTGGGCGGGAACGGGTTTTACCTAC | |
| | TGGGGACAGGGCACCCTTGTTACAGTGTCAAGT | |
| VH | EVQLVESGGGLVKPGGSLRLSCAASGFEFGSYEMNWVR | 119 |
| | QAPGKGLEWVSSISEDGYTTYYPDSLKGRFTISRDSAKNS | |
| | LYLQMNSLRADDTAVYYCARDFGGDTNWAGTGFTYWG | |
| | QGTLVTVSS | |
| VL Germline | VL1-40 | |
| VL FR1 | QSVLTQPPSVSGAPGQRITISC | 121 |
| VL CDR1 | EGSSSNIGAGYDVH | 122 |
| VL FR2 | WYQQLPGTAPKLLIY | 123 |
| VL CDR2 | GSSERNY | 124 |
| VL FR3 | GVPDRFSGSKSGTSASLAITGLQAEDEADYYC | 125 |
| VL CDR3 | QSYDSDLGVLYT | 126 |
| VL FR4 | FGTGTKVTVL | 127 |
| VL DNA | CAATCTGTACTTACACAGCCTCCCAGCGTGAGCGGTGC | 128 |
| | TCCAGGACAGAGGATCACTATTTCCTGTGAAGGCAGTT | |
| | CATCCAATATTGGTGCAGGATATGACGTTCATTGGTAC | |
| | CAACAACTTCCGGGAACCGCCCCAAAACTTTTAATCTA | |
| | TGGCAGCAGTGAGCGTAATTACGGTGTGCCTGATAGGT | |
| | TCTCCGGCTCTAAAAGCGGTACCAGTGCGAGTTTAGCC | |
| | ATCACAGGGCTACAAGCCGAGGATGAGGCCGATTATT | |
| | ATTGTCAAAGCTACGATAGCGATCTAGGCGTTTTGTAC | |
| | ACCTTCGGTACAGGCACGAAAGTCACTGTTCTT | |
| VL | QSVLTQPPSVSGAPGQRITISCEGSSSNIGAGYDVHWYQQ | 129 |
| | LPGTAPKLLIYGSSERNYGVPDRFSGSKSGTSASLAITGLQ | |
| | AEDEADYYCQSYDSDLGVLYTFGTGTKVTVL | |
| Antibody | ADI-90035 | SEQ ID NO. |
| VH Germline | VH3-21 | |
| VH FR1 | EVQLVESGGGLVKPGGSLRLSCAASG | 131 |
| VH CDR1 | FDFGSYEMN | 132 |
| VH FR2 | WVRQAPGKGLEWVS | 133 |
| VH CDR2 | SISEDGRSTYYPDSLKG | 134 |
| VH FR3 | RFTISRDSAKNSLYLQMNSLRADDTAVYYC | 135 |
| VH CDR3 | ARDFGGDTAWAGTGFTY | 136 |
| VH FR4 | WGQGTLVTVSS | 137 |
| VH DNA | GAGGTGCAGTTAGTCGAAAGCGGTGGAGGACTTGTTA | 138 |
| | AACCAGGAGGGTCACTAAGGTTGAGTTGCGCGGCTTC | |
| | AGGATTCGATTTCGGATCATACGAAATGAACTGGGTTC | |
| | GTCAAGCGCCGGGAAAAGGCTTGGAGTGGGTCAGTAG | |
| | TATCTCAGAGGATGGACGAAGCACGTATTATCCTGATT | |
| | CCTTGAAAGGTCGTTTTACAATAAGTAGGGATTCCGCT | |
| | AAAAACTCATTGTATCTTCAAATGAACTCCTTGAGGGC | |
| | TGACGACACGGCTGTGTATTACTGCGCGAGAGACTTTG | |
| | GTGGCGACACTGCGTGGGCGGGAACGGGTTTTACCTA | |
| | CTGGGGACAGGGCACCCTTGTTACAGTGTCAAGT | |
| VH | EVQLVESGGGLVKPGGSLRLSCAASGFDFGSYEMNWVR | 139 |
| | QAPGKGLEWVSSISEDGRSTYYPDSLKGRFTISRDSAKNS | |
| | LYLQMNSLRADDTAVYYCARDFGGDTAWAGTGFTYWG | |
| | QGTLVTVSS | |
| VL Germline | VL1-40 | |
| VL FR1 | QSVLTQPPSVSGAPGQRITISC | 141 |
| VL CDR1 | TGSSSNIGAGYDVH | 142 |
| VL FR2 | WYQQLPGTAPKLLIY | 143 |
| VL CDR2 | GSSSRNY | 144 |
| VL FR3 | GVPDRFSGSKSGTSASLAITGLQAEDEADYYC | 145 |
| VL CDR3 | QSYDSDLGVLYT | 146 |
| VL FR4 | FGTGTKVTVL | 147 |
| VL DNA | CAATCTGTACTTACACAGCCTCCCAGCGTGAGCGGTGC | 148 |
| | TCCAGGACAGAGGATCACTATTTCCTGTACTGGCAGTT | |
| | CATCCAATATTGGTGCAGGATATGACGTTCATTGGTAC | |
| | CAACAACTTCCGGGAACCGCCCCAAAACTTTTAATCTA | |
| | TGGCAGCAGTAGTCGTAATTATGGTGTGCCTGATAGGT | |
| | TCTCCGGCTCTAAAAGCGGTACCAGTGCGAGTTTAGCC | |
| | ATCACAGGGCTACAAGCCGAGGATGAGGCCGATTATT | |
| | ATTGTCAAAGCTACGATAGCGATCTAGGCGTTTTGTAC | |
| | ACCTTCGGTACAGGCACGAAAGTCACTGTTCTT | |
| VL | QSVLTQPPSVSGAPGQRITISCTGSSSNIGAGYDVHWYQQ | 149 |
| | LPGTAPKLLIYGSSSRNYGVPDRFSGSKSGTSASLAITGLQ | |
| | AEDEADYYCQSYDSDLGVLYTFGTGTKVTVL | |

1. An isolated antibody, or antigen-binding fragment thereof, which specifically binds to the spike protein of a coronavirus (“CoV-S”), wherein said antibody, or antigen-binding fragment thereof, comprises a heavy chain variable region (VH) comprising a VH CDR1 comprising SEQ ID NO: 12, a VH CDR2 comprising SEQ ID NO: 14, and a VH CDR3 comprising SEQ ID NO: 16, and a light chain variable region (VL) comprising a VL CDR1 comprising SEQ ID NO: 22, a VL CDR2 comprising SEQ ID NO: 24, and a VL CDR3 comprising SEQ ID NO: 26.

2. The isolated antibody, or antigen-binding fragment thereof, of claim 1, wherein the VH comprises SEQ ID NO: 19 and the VL comprises SEQ ID NO: 29.

3. The isolated antibody, or antigen-binding fragment thereof, of claim 1, wherein the VH consists of SEQ ID NO: 19 and the VL consists of SEQ ID NO: 29.

4. The isolated antibody, or antigen-binding fragment thereof, of claim 1, wherein the CoV-S is the spike protein of SARS-CoV (“SARS-CoV-S”) or the spike protein of SARS-CoV-2 (“SARS-CoV-2-S”).

5. The isolated antibody, or antigen-binding fragment thereof, of claim 4, wherein SARS-CoV-S comprises a sequence having at least 95% identity to the amino acid sequence of SEQ ID NO: 1, and wherein SARS-CoV-2-S comprises a sequence having at least 95% identity to the amino acid sequence of SEQ ID NO:5.

6. The isolated antibody, or antigen-binding fragment thereof, of claim 4, wherein the SARS-CoV-2-S is a B.1.1.7 variant, a B.1.351 variant, a B.1.1.28 variant, a B.1.429 variant, a P.1 variant, a B.1.617 variant, a B.1.617.2 variant, a C.37 variant, a 1.621 variant, a AY.1 variant, a 1.623 variant, a C.36 variant, a A.27 variant, a AV.1 variant, a B.1.1.482 variant, a B.1.1.523 variant, a B.1.427 variant, a AY.4 variant, a AY.11 variant, a D614G variant, a B.1.1.529/BA.1 variant, a BA.4/5 variant, a BA.4.6 variant, a BA.7 variant, a BQ.1 variant, a BQ.1.1 variant, a BA.2.75 variant, a BN.1 variant, a XBB variant, a XBB.1 variant, a XBB.1.5 variant, a BJ.1 variant, a BM.1.1.1 variant, a BA.2.3.20 variant, a BF.7 variant, a XBC variant, a CH.1.1 variant, a XBB.1.16 variant, a XBB.1.5.10 variant, a XBB.1.5.1 variant, a XBB.2.3 variant, a FL.1.5.1 variant, a XBB.1.5.CONV0817 variant, a XBB.1.5.70 variant, a BA.2.86 variant, a BA2.86 CONV1207 variant, a HV.1 variant, a HK.3 variant, a JN.1 variant, a JN.4 variant, a JD.1.1 variant, a BA.2 variant, a JN.1.11.1 variant, a GE.1.2.1 variant, a JN.1.13.1 variant, a KQ.1 variant, a KP.1.1 variant, a KP.3 variant, a KP.2 variant, a JN.1.50 variant, a KP.3.1.1 variant, a XEC variant, a LB.1 variant, a MV.1 variant, a LP.8.1 variant, a MC.10.2. variant, a LF.8 variant, a LP.8.1.2 variant, a FL.15.1.1 variant, or a LF.7.2.1 variant.

7. The isolated antibody, or antigen-binding fragment thereof, of claim 4, wherein the antibody, or antigen-binding fragment thereof, cross-reacts with SARS-CoV-S and SARS-CoV-2-S.

8. The isolated antibody, or antigen-binding fragment thereof, of claim 1, wherein the antibody, or antigen-binding fragment thereof, binds to CoV-S with a KD value of about 100 nM or lower.

9. The isolated antibody, or antigen-binding fragment thereof, of claim 4, wherein the antibody, or antigen-binding fragment thereof, neutralizes SARS-CoV and/or SARS-CoV-2 with an IC₅₀ of about 100 nM or lower.

10. The isolated antibody, or antigen-binding fragment thereof, of claim 1, wherein the antibody, or antigen-binding fragment thereof, have a Tm of at least about 62-70° C., about 63-70° C., or about 64-70° C.

11. The isolated antibody, or antigen-binding fragment thereof of claim 1, wherein the antibody, or antigen-binding fragment thereof, have a Ton of at least about 56-70° C., about 57-70° C., or about 58-70° C.

12. The isolated antibody, or antigen-binding fragment thereof of claim 1, wherein the antibody, or antigen-binding fragment thereof, has a serum half-life of about 40-200 days, about 50-160 days, about 50-140 days, about 55-75 days, about 60-90 days, about 50-90 days, about 50-80 days, about 40-70 days, or about 60-80 days.

13. The isolated antibody, or antigen-binding fragment thereof, of claim 1, wherein the antibody is human, humanized, primatized, chimeric, bispecific or multispecific.

14. The isolated antibody, or antigen-binding fragment thereof, of claim 1, wherein the antigen-binding fragment thereof comprises a Fab, Fab2, or scFv.

15. A pharmaceutical composition comprising the antibody, or antigen-binding fragment thereof, of claim 1, and a pharmaceutically acceptable carrier.

16. A pharmaceutical composition comprising the antibody, or antigen-binding fragment thereof, of claim 2, and a pharmaceutically acceptable carrier.

17. A pharmaceutical composition comprising the antibody, or antigen-binding fragment thereof, of claim 3, and a pharmaceutically acceptable carrier.

18. A method of treating or preventing infection by SARS-CoV, SARS-CoV-2, and/or another coronavirus in a subject in need thereof, comprising administering to the subject a therapeutically effective amount of an antibody, or antigen-binding fragment thereof, which specifically binds to a spike protein of the coronavirus,
wherein the antibody, or antigen-binding fragment thereof, comprises a heavy chain variable region (VH) comprising a VH CDR1 comprising SEQ ID NO: 12, a VH CDR2 comprising SEQ ID NO: 14, and a VH CDR3 comprising SEQ ID NO: 16, and a light chain variable region (VL) comprising a VL CDR1 comprising SEQ ID NO: 22, a VL CDR2 comprising SEQ ID NO: 24, and a VL CDR3 comprising SEQ ID NO: 26,
thereby treating or preventing infection by SARS-CoV, SARS-CoV-2, and/or another coronavirus in the subject.

19. The method of claim 18, wherein the VH comprises SEQ ID NO: 19 and the VL comprises SEQ ID NO: 29.

20. The method of claim 18, wherein the VH consists of SEQ ID NO: 19 and the VL consists of SEQ ID NO: 29.

21. The method of claim 18, wherein the subject is a human subject.

22. The method of claim 21, wherein the subject is an adult, or an adolescent at least 12 years of age or older and weighting at least 40 kg.

23. The method of claim 18, wherein the subject is immunocompetent.

24. The method of claim 18, wherein the subject is immunocompromised, or the subject has at least one risk factor which renders them more prone to a poor clinical outcome and/or progression to severe infection.

25. The method of claim 24, wherein the at least one risk factor is one or more of (i) an old age such as over 55, 60 or 65 years old, (ii) diabetes, (iii) a chronic respiratory condition such as asthma, bronchiectasis, cystic fibrosis, another fibrotic condition, chronic obstructive pulmonary disease (COPD), damaged or scarred lung tissue, pulmonary embolism or pulmonary hypertension, (iv) obesity, (iv) hypertension, (v) a cardiac or cardiovascular condition, such as heart defects or abnormalities, heart failure, coronary artery disease, cardiomyopathies, or hypertension, (vi) a chronic inflammatory or autoimmune condition such as lupus and multiple sclerosis, (vii) an immunocompromised status which may be caused by cancer, undergoing chemotherapy, smoking, bone marrow or organ transplantation, immune deficiencies, poorly controlled HIV infection or AIDS, or prolonged use of corticosteroids or other immunosuppressive medications, (viii) cerebrovascular disease or stroke, (ix) chronic kidney disease, (x) chronic liver disease, such as alcohol-related liver disease, non-alcoholic fatty liver disease, autoimmune hepatitis, or cirrhosis, (xi) Down syndrome, (xii) hemoglobin blood disorders, such as sickle cell disease and thalassemia, and (xiii) neurologic conditions such as dementia or Parkinson's disease.

26. The method of claim 18, wherein the antibody, or antigen-binding fragment thereof, is administered intravenously, subcutaneously, or intramuscularly.

27. The method of claim 18, wherein the antibody, or antigen-binding fragment thereof, is administered at a dose of about 250 mg to about 1000 mg intramuscularly.

28. The method of claim 18, wherein the antibody, or antigen-binding fragment thereof, is administered at a dose of about 1500 mg to about 4500 mg intravenously.

29. The method of claim 18, wherein the antibody, or antigen-binding fragment thereof, is administered at a dose of about 250 mg to about 1250 mg subcutaneously.

30. The method of claim 18, wherein the antibody, or antigen-binding fragment thereof, is administered once, or is administered weekly, monthly, every two months, every three months, every six months, or every year.
